(19) United States
(12) Patent Application Publication
(10) Pub. No.: US 2011/0218188 A1
(43) Pub. Date: Sep. 8, 2011

(54) SUBSTITUTED (PYRIDYL)-AZINYLAMINE DERIVATIVES AS FUNGICIDES

(76) Inventors: Christian Beier, Saint Genis-Laval (FR); Jurgen Benting, Leichlingen (DE); Isabelle Christian, Lyon (FR); Pierre-Yves Coqueron, Lyon (FR); Peter Dahmen, Neuss (DE); Ralf Dunkel, Leichlingen-Krawhwinkel (DE); Jorg Greul, Leichlingen (DE); Marie-Claire Grosjean-Cournoyer, Curis au Mont d'Or (FR); Hiroyuki Hadano, Tochigi (JP); Philippe Rinolfi, Chatillon d'Azergues (FR); Arnd Voerste, Koln (DE); Jean-Pierre Vors, Sainte-Foy-Les-Lyon (FR); Ulrike Wachendorff-Neumann, Oberer Markenweg (DE)

(21) Appl. No.: 13/129,021

(22) PCT Filed: Nov. 12, 2009
(86) PCT No.: PCT/EP09/65018

§ 371 Date: May 12, 2011

(30) Foreign Application Priority Data
Nov. 13, 2008 (EP) 08356139.9

(51) Int. Cl. A61K 31/55; C07D 401/14; A61K 31/506; A01P 3/00; A61K 31/5377; C07D 413/14; C07D 403/04; A61K 31/53; A61K 31/539

(52) U.S. Cl. 514/212.08; 544/331; 514/275; 540/524; 514/235.8; 544/122; 544/212; 514/245; 540/598; 514/217.05; 544/113; 514/236.2; 514/229.2; 544/65

(57) ABSTRACT

The present invention relates to phenyl-pyrimidinyl-amino derivatives of formula (I) wherein Q¹ and p, R^{a} to R^{c}, L¹, Y, L² and Q² represent various substituents, their process of preparation, preparation intermediate compounds, their use as fungicide active agents, particularly in the form of fungicide compositions, and methods for the control of phytopathogenic fungi, notably of plants, using these compounds or compositions.

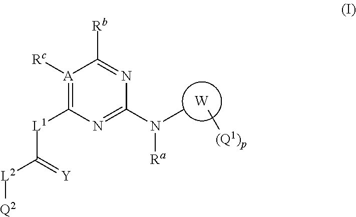

The present invention relates to substituted (pyridyl)-azinylamino derivatives, their process of preparation, preparation intermediate compounds, their use as fungicide active agents, particularly in the form of fungicide compositions, and methods for the control of phytopathogenic fungi, notably of plants, using these compounds or compositions.

WO 2007/003525 discloses N-Phenyl-triazinylamine derivatives useful as inhibitors of enzymes treating disease or disease symptoms. However, this reference does not relate to fungicidal applications of such derivatives. Additionally, WO 2005/019211 and WO 2005/033095 disclose a method of protecting plants against attack by phytopathogenic organisms using aminopyridinyl substituted N-Phenyl-triazinylamine derivatives. However, the said chemical structure of these compounds of the prior art is different from the compounds of the present invention.

It is always of high-interest in agriculture to use novel pesticide compounds in order to avoid or to control the development of resistant strains to the active ingredients. It is also of high-interest to use novel compounds being more active than those already known, with the aim of decreasing the amounts of active compound to be used, whilst at the same time maintaining effectiveness at least equivalent to the already known compounds. We have now found a new family of compounds which possess the above mentioned effects or advantages.

Accordingly, the present invention provides N-substituted (pyridyl)-azinyl-amino derivatives of formula (I)

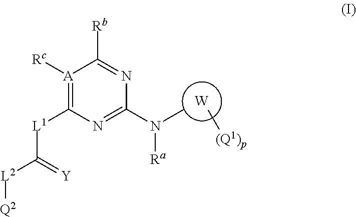

### Wherein

W represents phenyl or a saturated or unsaturated, aromatic or non-aromatic 4-, 5-, 6- or 7-membered heterocycle comprising up to four heteroatoms which may be the same or different
A represents a carbon atom or a nitrogen atom provided that if A represents a carbon atom then W represents a saturated or unsaturated, aromatic or non-aromatic 4-, 5-, 6- or 7-membered heterocycle comprising up to four heteroatoms which may be the same or different
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino; it being possible for each of these groups or substituents to be substituted when chemically possible;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, or a C₁-C₈-halogenoalkoxyalkyl having 1 to 5 halogen atoms, a C₁-C₈-alkoxyalkylcarbonyl, a C₁-C₈-halogenoalkoxyalkycarbonyl having 1 to 5 halogen atoms, a C₁-C₈-alkylthioalkylcarbonyl, a C₁-C₈-halogenoalkylthioalkylcarbonyl having 1 to 5 halogen atoms; it being possible for each of these groups or substituents to be substituted when chemically possible;
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, or a C₁-C₈-halogenocycloalkyl having 1 to 5 halogen atoms; it being possible for each of these groups or substituents to be substituted when chemically possible;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible;
Alternatively, L² and Q² can form together a substituted or non-substituted 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible;
   as well as salts, N-oxides, metallic complexes, metalloïdic complexes and optically active or geometric isomers thereof; provided that the following compounds are excluded:
(3-{[4-(2-ethenylpyridin-4-yl)-1,3,5-triazin-2-yl]amino}phenyl)methanol;
methyl 4-{4-[(3-{2-[(tert-butoxycarbonyl)amino]ethoxy}phenyl)amino]-1,3,5-triazin-2-yl}-pyridine-2-carboxylate;
N-{3-[(4-{2-[(1Z)-3-amino-2-chloroprop-1-en-1-yl]pyridin-4-yl}-1,3,5-triazin-2-yl)amino]-benzyl}-N-methylglycine.
   In a particular embodiment of the invention, compounds of formula (I) according to the invention are those wherein A represents a nitrogen atom and W represents phenyl. A compound of formula (I) according to the invention is then represented by a compound of the Formula II:

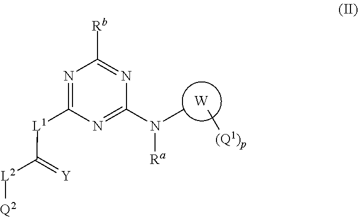

wherein
W represents phenyl
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbarbonyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₈-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino; it being possible for each of these groups or substituents to be substituted when chemically possible;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms, a C₁-C₈-alkoxyalkylcarbonyl, a C₁-C₈-halogenoalkoxyalkycarbonyl having 1 to 5 halogen atoms, a C₁-C₈-alkylthioalkylcarbonyl, a C₁-C₈-halogenoalkylthioalkylcarbonyl having 1 to 5 halogen atoms; it being possible for each of these groups or substituents to be substituted when chemically possible;
R^{b} represents a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms; it being possible for each of these groups or substituents to be substituted when chemically possible;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible
Alternatively, L2 and Q2 can form together a substituted or non-substituted, 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible
   as well as salts, N-oxides, metallic complexes, metalloïdic complexes and optically active or geometric isomers thereof.

In another particular embodiment of the invention, compounds of formula (I) according to the invention are those wherein W represents a saturated or unsaturated, aromatic or non-aromatic 4-, 5-, 6- or 7-membered heterocycle comprising up to four heteroatoms which may be the same or different.

A compound of formula (I) according to the invention is then represented by a compound of the Formula (III):

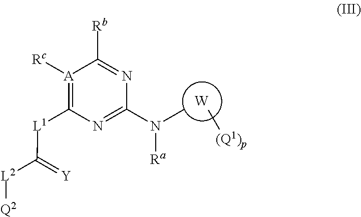

wherein
W represents a saturated or unsaturated, aromatic or non-aromatic 4-, 5-, 6- or 7-membered heterocycle comprising up to four heteroatoms which may be the same or different
A represents a carbon atom or a nitrogen atom
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino; it being possible for each of these groups or substituents to be substituted when chemically possible;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms, a C₁-C₈-alkoxyalkylcarbonyl, a C₁-C₈-halogenoalkoxyalkycarbonyl having 1 to 5 halogen atoms, a C₁-C₈-alkylthioalkylcarbonyl, a C₁-C₈-halogenoalkylthioalkylcarbonyl having 1 to 5 halogen atoms; it being possible for each of these groups or substituents to be substituted when chemically possible;
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms; it being possible for each of these groups or substituents to be substituted when chemically possible;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible;
Alternatively, L2 and Q2 can form together a substituted or non-substituted, 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible;
   as well as salts, N-oxides, metallic complexes, metalloïdic complexes and optically active or geometric isomers thereof.

In another particular embodiment of the invention, compounds of formula (III) according to the invention are those wherein A represents a nitrogen atom.

A compound of formula (I) according to the invention is then represented by a compound of the Formula (III₁):

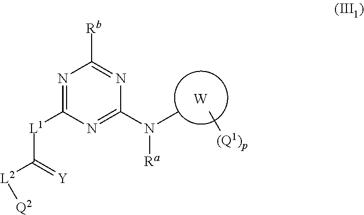

wherein
W represents a saturated or unsaturated, aromatic or non-aromatic 4-, 5-, 6- or 7-membered heterocycle comprising up to four heteroatoms which may be the same or different
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₈-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino; it being possible for each of these groups or substituents to be substituted when chemically possible;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms, a C₁-C₈-alkoxyalkylcarbonyl, a C₁-C₈-halogenoalkoxyalkycarbonyl having 1 to 5 halogen atoms, a C₁-C₈-alkylthioalkylcarbonyl, a C₁-C₈-halogenoalkylthioalkylcarbonyl having 1 to 5 halogen atoms; it being possible for each of these groups or substituents to be substituted when chemically possible;
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms; it being possible for each of these groups or substituents to be substituted when chemically possible;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible;
Alternatively, L2 and Q2 can form together a substituted or non-substituted, 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible;
   as well as salts, N-oxides, metallic complexes, metalloïdic complexes and optically active or geometric isomers thereof.

In another particular embodiment of the invention, compounds of formula (III) according to the invention are those wherein A represents a carbon atom.

A compound of formula (I) according to the invention is then represented by a compound of the Formula (III₂)

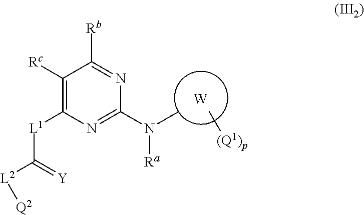

wherein
W represents a saturated or unsaturated, aromatic or non-aromatic 4-, 5-, 6- or 7-membered heterocycle comprising up to four heteroatoms which may be the same or different
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino; it being possible for each of these groups or substituents to be substituted when chemically possible;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms, a C₁-C₈-alkoxyalkylcarbonyl, a C₁-C₈-halogenoalkoxyalkycarbonyl having 1 to 5 halogen atoms, a C₁-C₈-alkylthioalkylcarbonyl, a C₁-C₈-halogenoalkylthioalkylcarbonyl having 1 to 5 halogen atoms; it being possible for each of these groups or substituents to be substituted when chemically possible;
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms; it being possible for each of these groups or substituents to be substituted when chemically possible;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible;
Alternatively, L2 and Q2 can form together a substituted or non-substituted, 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible;
   as well as salts, N-oxides, metallic complexes, metalloïdic complexes and optically active or geometric isomers thereof.

Any of the compounds according to the present invention may exist in one or more optical or chiral isomeric form depending on the number of asymmetric centres in the compound. The invention thus relates equally to all optical isomers and to any racemic or scalemic mixtures thereof (the term “scalemic” denotes a mixture of enantiomers in different proportions), and to the mixtures of any potential stereoisomers, in any proportion. Diastereoisomers or optical isomers can be separated according to any methods known per se by the man ordinary skilled in the art.

Any of the compounds according to the present invention may also exist in one or more geometric isomeric form depending on the number of double bond within the compound. The invention thus equally relates to any geometric isomer and to any possible mixtures thereof, in any proportion. Geometric isomers can be separated according to any method known per se by the man ordinary skilled in the art.

Any compound of formulas (I, II, III, III₁, III₂) according to the invention wherein L²Q² represents a hydroxy group, a sulfanyl group or an amino group can exist in a tautomeric form resulting from the shift of the proton of said hydroxy group, sulfanyl group or amino group respectively. Such tautomeric forms are also part of the present invention. Generally, any tautomeric form of a compound of formulas (I, II, III, III₁, III₂) according to the invention wherein L²Q² represents a hydroxy group, a sulfanyl group or an amino group, as well as the tautomeric forms of the compounds which can optionally be used as intermediates in the preparation processes according to the invention are also part of the present invention.

According to the invention, the following generic terms are generally used with the following meanings:
halogen means fluorine, chlorine, bromine or iodine;
heteroatom can be nitrogen, oxygen or sulphur;
unless indicated otherwise, a group or a substituent that is substituted according to the invention can be substituted by one or more of the following groups or atoms: a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a to C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino.

Preferred compounds of formula (I) according to the invention are those wherein W represents phenyl.

Other preferred compounds of formula (I) according to the invention are those wherein W represents a saturated or unsaturated, aromatic or non-aromatic heterocycle selected in the list consisting of:

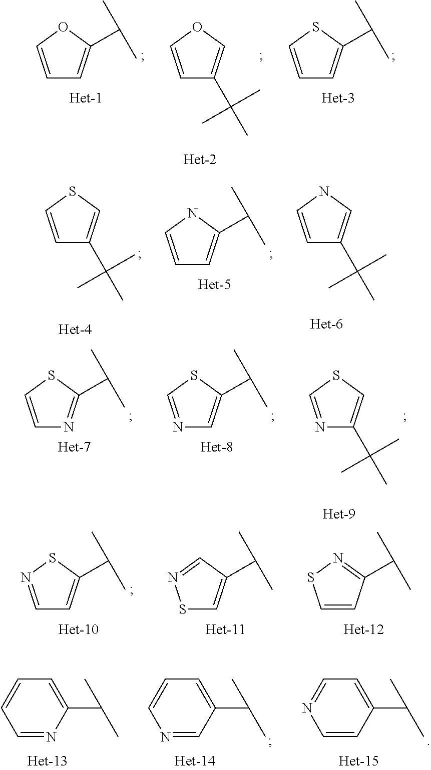

Other preferred compounds of formula (I) according to the invention are those wherein Q¹ represents a halogen atom, a nitro group, a hydroxy group, a cyano group, an amino group, a sulfanyl group, a pentafluoro-λ⁶-sulfanyl group, a formyl group, a formyloxy group, a formylamino group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₁-C₈-alkylcarbonyl, a C₁-C₈-halogenoalkylcarbonyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a (C₁-C₆-alkoxyimino)-C₁-C₆-alkyl, C₁-C₈-alkoxyalkyl, C₁-C₈-halogenoalkoxyalkyl having 1 to 5 halogen atoms; it being possible for each of these groups or substituents to be substituted when chemically possible.

Other preferred compounds of formula (I) according to the invention are those wherein p represents 0, 1, 2, or 3. More preferably, p represents 0 or 1. Even more preferably p represents 1.

Other preferred compounds of formula (I) according to the invention are those wherein R^{a} represents a hydrogen atom or a substituted or non substituted C₁-C₈-cycloalkyl.

Other preferred compounds of formula (I) according to the invention are those wherein R^{b} and R^{c} independently represent a hydrogen atom, a halogen atom, a cyano, a C₁-C₈-halogenoalkyl having 1 to 5 halogen atoms, a C₁-C₈-halogenocycloalkyl having 1 to 5 halogen atoms. More preferably, R^{b} and R^{c} independently represent a hydrogen atom or a halogen atom.

Other preferred compounds of formula (I) according to the invention are those wherein L¹ is selected in the list consisting of:

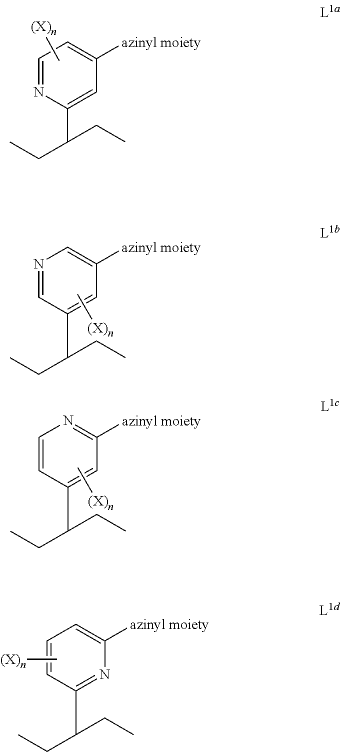

wherein
n represents 0, 1, 2 or 3;
X independently represents a C₁-C₁₀-alkyl, a C₁-C₁₀-halogenoalkyl, a halogen atom or a cyano.

More preferred compounds of formula (I) according to the invention are those wherein L¹ represents

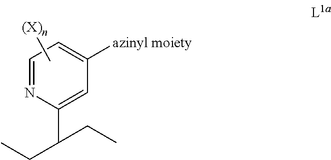

wherein
n represents 0, 1, 2 or 3;
X independently represents a C₁-C₁₀-alkyl, a C₁-C₁₀-halogenoalkyl, a halogen atom or a cyano.

Even more preferred compounds of formula (I) according to the invention are those wherein L¹ represents

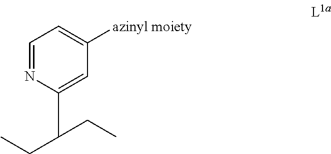

Other preferred compounds of formula (I) according to the invention are those wherein Q² represents a hydrogen atom, a halogen atom, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, C₁-C₈-cycloalkyl, C₁-C₈-halogenoalkyl having 1 to 5 halogen atoms, a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-alkylcarbonyl, C₁-C₈-halogenoalkylcarbonyl having 1 to 5 halogen atoms, C₁-C₈-halogenoalkoxycarbonyl having 1 to 5 halogen atoms, C₁-C₈-alkylcarbonylamino, C₁-C₈-halogenoalkylcarbonylamino having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible;

When L² and Q² form together a, 4-, 5-, 6- or 7-membered heterocycle comprising up to 4 heteroatoms selected in the list consisting of N, O, S, preferred resulting heterocycles are non-aromatic. More preferred heterocycles are pyrrolidine, piperidine, morpholine.

Other preferred compounds of formula (I) according to the invention are those wherein R^{d} to Rⁱ independently represent a hydrogen atom, a halogen atom, a nitro group, a cyano group, a hydroxy group, an amino group, a sulfanyl group, a formyl group, a formyloxy group, a formylamino group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino or a 4-, 5-, 6- or 7-membered heterocycle comprising up to 4 heteroatoms selected in the list consisting of N, O, S; it being possible for each of these groups or substituents to be substituted when chemically possible.

Other more preferred compounds of formula (I) according to the invention are those wherein R^{d} represents H, (methoxycarbonyl)amino, (4-chlorophenyl)amino, [3-chloro-5-(trifluoromethyl)pyridin-2-yl]amino, (2-ethoxy-2-oxoethyl)amino, (2,2,2-trifluoroethyl)amino, (2-cyanoethyl)amino, methylamino, (2-methylpropanoyl)oxy, (3-methylbut-2-enoyl)oxy, (3-methylbutanoyl)oxy, butanoyloxy, propanoyloxy, (methoxyacetyl)oxy, acetyloxy, cyclopentyloxy, dicyclopropylmethoxy, 1-cyclopropylethoxy, but-3-yn-2-yloxy, hex-2-yn-1-yloxy, but-2-yn-1-yloxy, prop-2-yn-1-yloxy, 2,2,2-trifluoroethoxy, (2,6-dichlorobenzyl)oxy, (4-chlorobenzyl)oxy, (4-methoxybenzyl)oxy, benzyloxy, cyclopropylmethoxy, 2-methylpropoxy, prop-2-en-1-yloxy, propoxy, 2-(dimethylamino)ethoxy, ethoxy, methoxy, hydroxyl, phenylamino, or phenyl hetarylamino.

Other more preferred compounds of formula (I) according to the invention are those wherein R^{g} represents Hydrogen, prop-2-en-1-yl, hexyl, butyl, propyl, 2-hydroxyethyl, ethyl, methyl.

Other more preferred compounds of formula (I) according to the invention are those wherein Q² represents (2R)-2-(methoxymethyl)pyrrolidin-1-yl, (2S)-1-methoxypropan-2-yl, 1-(diethylamino)propan-2-yl, 1-(dimethylamino)propan-2-yl, 1,1-dioxidotetrahydrothiophen-3-yl, 1,3-dimethoxypropan-2-yl, 1-cyanobutan-2-yl, 1-cyclopropyl-2-methoxyethyl, 1-ethylpiperidin-3-yl, 1-methoxybutan-2-yl, 1-methoxypropan-2-yl, 2-(hydroxymethyl)piperidin-1-yl, 2-(morpholin-4-yl)ethyl, 2,2,2-trifluoroethyl, 2,3-dimethylpiperidin-1-yl, 2,5-dimethylpyrrolidin-1-yl, 2,6-dimethylmorpholin-4-yl, 2-cyanoethyl, 2-ethylpiperidin-1-yl, 2-hydroxy-2-methylpropyl, 2-hydroxyethyl, 2-methoxyethyl, 2-methylpiperidin-1-yl, 2-methylprop-2-en-1-yl, 2-methylpropyl, 2-methylpyrrolidin-1-yl, 3-(2-oxoazepan-1-yl)propyl, 3-(2-oxopyrrolidin-1-yl)propyl, 3-(formylamino)propyl, 3-(hydroxymethyl)piperidin-1-yl, 3,3,3-trifluoropropyl, 3,3-dimethylpiperidin-1-yl, 3,5-dimethylpiperidin-1-yl, 3,6-dihydropyridin-1(2H)-yl, 3-hydroxypiperidin-1-yl, 3-hydroxypropyl, 3-hydroxypyrrolidin-1-yl, 3-methoxybutan-2-yl, 3-methoxypiperidin-1-yl, 3-methoxypropyl, 3-methylbut-2-en-1-yl, 3-methylbutan-2-yl, 3-methylbutyl, 3-methylpiperidin-1-yl, 4-(2-oxopyrrolidin-1-yl)butyl, 4-(trifluoromethyl)piperidin-1-yl, 4-cyanopiperidin-1-yl, 4-ethoxycyclohexyl, 4-formylpiperazin-1-yl, 4-hydroxypiperidin-1-yl, 4-methoxypiperidin-1-yl, 4-methylpiperazin-1-yl, 4-methylpiperidin-1-yl, 4-oxoimidazolidin-1-yl, azepan-1-yl, butan-2-yl, butyl, cyclobutyl, cyclohexyl, cyclopentyl, cyclopropyl, cyclopropylmethyl, ethyl, hydrogen, hexyl, hydroxy, methoxy, methyl, morpholin-4-yl, oxetan-3-yl, pentan-2-yl, pentan-3-yl, pentyl, piperidin-1-yl, prop-2-en-1-yl, propan-2-yl, propyl, pyrrolidin-1-yl, tert-butyl, tetrahydrofuran-2-ylmethyl, thiomorpholin-4-yl.

The above mentioned preferences with regard to the substituents of the compounds of formula (I) according to the invention can be combined in various manners, either individually, partially or entirely. These combinations of preferred features thus provide sub-classes of compounds according to the invention. Examples of such sub-classes of preferred compounds according to the invention can combine:
preferred features of W with preferred features of one or more of Q¹ and p, R^{a} to Rⁱ, L¹, Y, L² and Q²;
preferred features of Q¹ and p with preferred features of one or more of W, R^{a} to Rⁱ, L¹, Y, L² and Q²;
preferred features of R^{a} to Rⁱ with preferred features of one or more of W, Q¹ and p, L¹, Y, L² and Q²;
preferred features of L¹ with preferred features of one or more of W, Q¹ and p, R^{a} to Rⁱ, Y, L² and Q²;
preferred features of Y with preferred features of one or more of W, Q¹ and p, R^{a} to Rⁱ, L¹, L² and Q²;
preferred features of L² with preferred features of one or more of W, Q¹ and p, R^{a} to Rⁱ, L¹, Y and Q²;
preferred features of Q² with preferred features of one or more of W, Q¹ and p, R^{a} to Rⁱ, L¹, Y and L².

In these combinations of preferred features of the substituents of the compounds according to the invention, the said preferred features can also be selected among the more preferred features of each of W, Q¹ and p, R^{a} to Rⁱ, L¹, Y, L² and Q² so as to form most preferred subclasses of compounds according to the invention.

The preferred features of the other substituents of the compounds according to the invention can also be part of such sub-classes of preferred compounds according to the invention, notably the groups of substituents W, Q¹ and p, R^{a} to Rⁱ, L¹, Y, L² and Q².

The present invention also relates to a process for the preparation of compounds of formula (I). Thus according to a further aspect of the present invention, there is provided a process P1 for the preparation of a compound of formula (I) as herein-defined, as illustrated by the following reaction scheme:

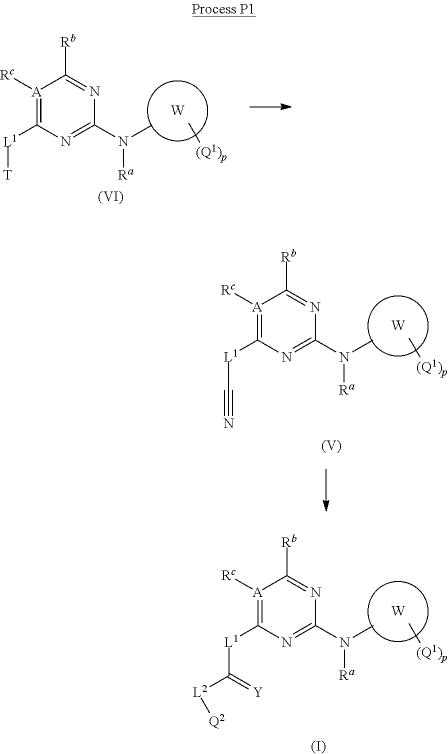

wherein
T represents a leaving group such as a halogen atom, a C₁-C₆alkylsulfonate, a C₁-C₆ haloalkylsulfonate; a substituted or non-substitued phenylsulfonate and
if Y represents an oxygen atom and L² represents CR^{h}Rⁱ;
A, W, Q¹, p, R^{a}, R^{b}, R^{c}, R^{h}, Rⁱ, L¹, Q² being as herein-defined; and that comprises
   reacting a compound of formula (VI) with a cyanide reagent such as a metallic cyanide for example sodium cyanide, potassium cyanide, zinc cyanide; a to metalloklic cyanide, an organo-metallic cyanide for example di-C₁-C₆-alkylaluminum cyanide notably di-ethylaluminum cyanide; an organo-metalloklic cyanide for example tri-C₁-C₆-alkylsilylcyanide notably tri-methylsilylcyanide in order to yield a compound of formula (V), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (V)
   then reacting said compound of formula (V) with an organo-metallic reagent of formula Q²-L²-M, wherein M represents a metal such as lithium, magnesium, sodium, potassium or a metallic salt such as magnesium salt, lithium salt, potassium salt or sodium salt; to yield a compound of formula (I); optionally in the presence of a catalyst;
   or by then reacting said compound of formula (V) with a phosphorane ylide reagent of formula Q²-L²-U, wherein U represents a tri-(phenyl)-phosphonium group, a di-(C₁-C₆)-alkylphosphonate; to yield a compound of formula (I); in the presence of a base, such as an inorganic or an organic base; preferably an alkaline earth metal or alkali metal hydrides, hydroxides, amides, alcoholates, acetates, carbonates or hydrogen carbonates, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); optionally in the presence of a catalyst;
if Y represents an oxygen atom, L² represents a direct bond and Q² represents a hydrogen atom;
A, W, Q¹, p, R^{a}, R^{b}, R^{c}, L¹, being as herein defined; and that comprises
   reacting a compound of formula (VI) with a cyanide reagent such as a metallic cyanide for example sodium cyanide, potassium cyanide, zinc cyanide; a metalloklic cyanide, an organo-metallic cyanide for example di-C₁-C₆-alkylaluminum cyanide notably di-ethylaluminum cyanide; an organo-metalloklic cyanide for example tri-C₁-C₆-alkylsilylcyanide notably tri-methylsilylcyanide in order to yield a compound of formula (V), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (V),
   reacting said compound of formula (V) with a reducing agent such as hydrogen, a metal, such as magnesium, a metallic salt such as SnCl₂ or SnBr₂; or a hydride donor of formula H-M, wherein M represents a metal, or a metallic salt, such as di-C₁-C₆-alkylaluminum hydrides, notably di-ethylaluminum hydride, to yield a compound of formula (I); optionally in the presence of a catalyst;
if Y represents an oxygen atom, L² represents an oxygen atom and Q² represents a hydrogen atom;
A, W, Q¹, p, R^{a}, R^{b}, R^{c}, L¹, being as herein defined; and that comprises
   reacting a compound of formula (VI) with a cyanide reagent such as a metallic cyanide for example sodium cyanide, potassium cyanide, zinc cyanide; a metalloklic cyanide, an organo-metallic cyanide for example di-C₁-C₆-alkylaluminum cyanide notably di-ethylaluminum cyanide; an organo-metalloklic cyanide for example tri-C₁-C₆-alkylsilylcyanide notably tri-methylsilylcyanide in order to yield a compound of formula (V), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (V),
   hydrolyzing said compound of formula (V), preferably in presence of water, optionally in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, or an organic acid, such as para-toluenesulphonic acid; to yield a compound of formula (I);
if Y represents NH, L² represents a direct bond, a sulphur atom, an oxygen atom or NH, and Q² represents a hydrogen atom, a halogen atom, a nitro group, a hydroxy group, a cyano group, an amino group, a sulfanyl group, a formyloxy group, a formylamino group, a carbamate group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylamino, phenoxy or phenylamino;
A, W, Q¹, p, R^{a}, R^{b}, R^{c}, L¹, being as herein defined; and that comprises
   reacting a compound of formula (VI) with a cyanide reagent such as a metallic cyanide for example sodium cyanide, potassium cyanide, zinc cyanide; a metalloklic cyanide, an organo-metallic cyanide for example di-C₁-C₆-alkylaluminum cyanide notably di-ethylaluminum cyanide; an organo-metalloklic cyanide for example tri-C₁-C₆-alkylsilylcyanide notably tri-methylsilylcyanide in order to yield a compound of formula (V), optionally in the presence of a catalyst, notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (V),
   performing the addition reaction of a compound of formula (V) with a reagent of formula Q²-L²-H, optionally in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); optionally in the presence of an acid such as a Lewis acid; notably metal or metalloïd halides such as aluminium trichloride, zinc dichloride, magnesium bromide boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid; optionally in the presence of a catalyst, to yield a compound of formula (I).

Advantageously, process P1 according to the invention can be simplified, allowing the direct preparation of certain compounds of formula (I) starting from a compound of formula (VI). Accordingly, the present invention provides an improved process P1A for the preparation of a compound of formula (I), as illustrated by the following reaction scheme:

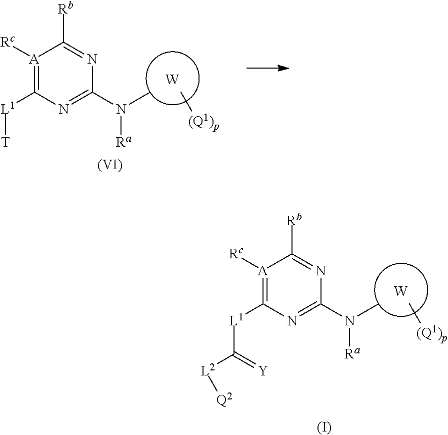

### Process P1A

wherein
T represents a leaving group such as a halogen atom, a C₁-C₆ alkylsulfonate, a C₁-C₆ haloalkylsulfonate; a substituted or non-substituted phenylsulfonate and
if Y represents an oxygen atom, L² represents an oxygen atom, a NR^{g} group and R^{g} represents a hydrogen atom, a nitro group, a cyano group, a hydroxy group, an amino group, a formyloxy group, a formylamino group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, or a 4-, 5-, 6- or 7-membered heterocycle comprising up to 4 heteroatoms selected in the list consisting of N, O, S;
A, W, Q¹, p, R^{a}, R^{b}, R^{c}, L¹, Q² being as herein-defined; and that comprises
   reacting a compound of formula (VI) with a compound of formula Q²-L²-H, wherein Q² is herein-defined and L² represents oxygen atom, a NR^{g} group and R^{g} represents a hydrogen atom, a nitro group, a cyano group, a hydroxy group, an amino group, a formyloxy group, a formylamino group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, (C₁-C₆-alkoxyimino)-C₁-C₆-alkyl, (C₁-C₈-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) (2-oxoazepan-1-yl) C₁-C₈-halogenoalkyl having 1 to 5 halogen atoms, (benzyloxyimino)-C₁-C₆-alkyl, or a 4-, 5-, 6- or 7-membered heterocycle comprising up to 4 heteroatoms selected in the list consisting of N, O, S; in the presence of carbon monoxide or a carbon monoxide generating agent such as Mo(CO)₆ or W(CO)₆, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine, optionally in the presence of a base such as an inorganic or an organic base; preferably an acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), to yield a compound of formula (I).

The process according to the invention also allows the preparation of compounds of formula (I) according to the invention using other compounds of formula (I) according to the invention as starting material.

Thus according to a further aspect of the present invention, there is provided a process P2 for the preparation of a compound of formula (I) wherein Y represents a NR^{d} group, L² represents CR^{h}Rⁱ; A, W, Q¹, p, R^{a}, R^{b}, R^{c}, R^{d}, R^{h}, Rⁱ, L¹, Q² being as herein defined;

that comprises reacting a different compound of formula (I) wherein Y represents an oxygen atom and L² represents CR^{h}Rⁱ; A, W, Q¹, p, R^{a}, R^{b}, R^{c}, R^{h}, Rⁱ, L¹, Q² being as herein-defined; with a compound of formula R^{d}NH or one of its salts, wherein R^{d} is as herein-defined, optionally in the presence of a dehydrating agent such as molecular sieves, anhydrous metal salts, such as magnesium sulphate, sodium sulphate, or metal oxides such as barium oxide, calcium oxide, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P3 for the preparation of a compound of formula (I) wherein Y represents a CR^{e}R^{f} group, L² represents an oxygen atom and Q² represents a formyl group, a (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, a or a 4-, 5-, 6- or 7-membered heterocycle comprising up to 4 heteroatoms selected in the list consisting of N, O, S; A, W, Q¹, p, R^{a}, R^{b}, R^{c}, R^{h}, Rⁱ, L¹, being as herein defined;

that comprises reacting a different compound of formula (I) wherein Y represents an oxygen atom, L² represents CR^{h}R¹ and Q² represents a hydrogen atom; A, W, Q¹, p, R^{a}, R^{b}, R^{c}, R^{h}, Rⁱ, L¹, being as herein-defined;
with a compound of formula Q²T wherein T represents a leaving group such as a halogen atom, a C₁-C₆ alkylsulfonate, a C₁-C₆ haloalkylsulfonate and Q² represents a formyl group, a (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, (C₁-C₈-alkoxyimino)-C₁-C₈-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, a or a 4-, 5-, 6- or 7-membered heterocycle comprising up to 4 heteroatoms selected in the list consisting of N, O, S; optionally in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU).

According to a further aspect of the present invention, there is provided a process P4 for the preparation of a compound of formula (I) wherein Y represents a NR^{d} group, L² represents a direct bond and Q² represents a hydrogen atom; A, W, Q¹, p, R^{a}, R^{b}, R^{c}, R^{d}, L¹ being as herein defined,

and that comprises reacting a different compound of formula (I) wherein Y represents an oxygen atom, L² represents a direct bond and Q² represents a hydrogen atom; A, W, Q¹, p, R^{a}, R^{b}, R^{c}, L¹ being as herein-defined;
with a compound of formula R^{d}NH or one of its salts, R^{d} being as herein-defined, optionally in the presence of dehydrating agent such as molecular sieves, anhydrous metal salts, such as magnesium sulphate, sodium sulphate, or metal oxides such as barium oxide, calcium oxide, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P5 for the to preparation of a compound of formula (I) wherein Y represents a CR^{e}R^{f} group, L² represents a direct bond and Q² represents a hydrogen atom; A, W, Q¹, p, R^{a}, R^{b}, R^{c}, L¹ being as herein defined

and that comprises reacting a different compound of formula (I) wherein Y represents an oxygen atom, L² represents a direct bond and Q² represents a hydrogen atom; A, W, Q¹, p, R^{a}, R^{b}, R^{c}, L¹ being as herein-defined;
with a compound of formula CHUR^{e}R^{f}, wherein U represents a hydrogen atom, a phosphonium group, a di-(C₁-C₆)-alkylphosphonate, R^{e}, R^{f} being as herein-defined, or one of its salts, a tri-(C₁-C₆)-alkylsilyl, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P6 for the preparation of a compound of formula (I) wherein Y represents a CR^{e}R^{f} group, L² represents CR^{h}Rⁱ and A, W, Q¹, p, R^{a}, R^{b}, R^{c}, R^{e}, R^{f}, Rⁱ, L¹, Q² being as herein-defined;

and that comprises reacting a different compound of formula (I) wherein Y represents an oxygen atom and, L² represents CR^{h}Rⁱ; A, W, Q¹, p, R^{a}, R^{b}, R^{c}, R^{e}, R^{f}, R^{h}, Rⁱ, L¹, Q² being as herein-defined;
with a compound of formula CHUR^{e}R^{f} wherein U represents a hydrogen atom, a tri-(phenyl)-phosphonium group, a di-(C₁-C₆)-alkylphosphonate, R^{e}, R^{f} being as herein-defined, or one of its salts, a tri-(C₁-C₆)-alkylsilyl, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P7 for the preparation of a compound of formula (I) wherein Y represents a NR^{d} group, L² represents an oxygen atom a sulphur atom or a NR^{g} group wherein R^{g} represents a hydrogen atom, a formyl group, a formyloxy group, a formylamino group, a carbamoyl group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₈-alkoxyimino)-C₁-C₈-alkyl, (C₁-C₈-alkenyloxyimino)-C₁-C₈-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₈-alkyl, C₁-C₈-alkoxyalkyl, C₁-C₈-halogenoalkoxyalkyl having 1 to 5 halogen atoms or a 4-, 5-, 6- or 7-membered heterocycle comprising up to 4 heteroatoms selected in the list consisting of N, O, S; Q² represents a formyl group, a (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, (C₁-C₆-alkoxyimino)-C₁-C₆-alkyl, (C₁-C₈-alkenyloxyimino)-C₁-C₈-alkyl, (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, a or a 4-, 5-, 6- or 7-membered heterocycle comprising up to 4 heteroatoms selected in the list consisting of N, O, S; and A, W, Q¹, p, R^{a}, R^{b}, R^{c}, R^{d}, L¹ being as herein-defined;

and that comprises reacting a different compound of formula (I) wherein Y represents a NR^{d} group, L² represents an oxygen atom, a sulphur atom or a NR^{g} group wherein R^{g} represents a hydrogen atom, a formyl group, a formyloxy group, a formylamino group, a carbamoyl group, (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms or a 4-, 5-, 6- or 7-membered heterocycle comprising up to 4 heteroatoms selected in the list consisting of N, O, S; Q² represents a hydrogen atom; A, W, Q¹, p, R^{a}, R^{b}, R^{c}, R^{d}, L¹, being as herein-defined;
with a compound of formula Q²T wherein T represents a leaving group such as a halogen atom, a C₁-C₆ alkylsulfonate, a C₁-C₆ haloalkylsulfonate and Q² represents a formyl group, a (hydroxyimino)-C₁-C₆-alkyl group, C₁-C₈-alkyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, a or a 4-, 5-, 6-, or 7-membered heterocycle comprising up to 4 heteroatoms selected in the list consisting of N, O, S; optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2,-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine.

According to a further aspect of the present invention, there is provided a process P8 for the preparation of a compound of formula (I) wherein Y represents a NR^{d} group wherein R^{d} represents a formyloxy group, a formylamino group, C₁-C₈-alkyl amino, C₁-C₈-cycloalkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₂-C₈-halogenoalkenyloxy having 1 to 5 halogen atoms, C₃-C₈-halogenoalkynyloxy having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkoxycarbonylamino, C₁-C₈-halogenoalkoxycarbonylamino having 1 to 5 halogen atoms, C₁-C₈-alkoxycarbonyloxy, C₁-C₈-halogenoalkoxycarbonyloxy having 1 to 5 halogen atoms; L² represents an oxygen atom, a sulphur atom or a NR^{g} group; Q² represents a hydrogen atom; and A, W, Q¹, p, R^{a}, R^{b}, R^{c}, R^{g}, L¹, being as herein-defined,

and that comprises reacting a different compound of formula (I) wherein Y represents a NR^{d} group wherein R^{d} represents an amino group, a hydroxy group C₁-C₈-alkylamino, C₁-C₈-cycloalkylamino, L² represents an oxygen atom, a sulphur atom or a NR^{g} group; Q² represents a hydrogen atom; A, W, Q¹, p, R^{a}, R^{b}, R^{c}, R^{g}, L¹, being as herein-defined;
with a compound of formula Q²T wherein T represents a leaving group such as a halogen atom, a C₁-C₆ alkylsulfonate, a C₁-C₆ haloalkylsulfonate and Q² represents a formyl group, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₂-C₈-halogenoalkenyl having 1 to 5 halogen atoms, C₃-C₈-halogenoalkynyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms; optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine.

According to a further aspect of the present invention, there is provided a process P9 for the preparation of a compound of formula (I) wherein Y represents an oxygen atom, L² represents an oxygen atom, a NR^{g} group; A, W, Q¹, p, R^{a}, R^{b}, R^{c}, R^{g}, L¹, Q², being as herein defined;

and that comprises reacting a different compound of formula (I) wherein Y represents an oxygen atom, L² represents an oxygen atom and Q² represents a hydrogen atom, a formyl group, a (hydroxyimino)-C₁-C₆-alkyl group, C₁-C₈-alkyl, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, a or a 4-, 5-, 6- or 7-membered heterocycle comprising up to 4 heteroatoms selected in the list consisting of N, O, S; A, W, Q¹, p, R^{a}, R^{b}, R^{c}, L¹, being as herein defined;
with a compound of formula Q²L²H wherein Q² being as herein defined and L² represents an oxygen atom, a NR^{g} group,
optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsu lfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; in one or a different-pot conditions.

According to the present invention, the compounds of formula (I) useful as starting material within the processes P2 to P9 can be prepared according to process P1 according to the invention.

According to a further aspect of the present invention, there is provided a process P10 for the preparation of a compound of formula (I) wherein Y represents a sulphur atom, L² represents an oxygen atom, a NR^{g} group and A, W, Q¹, p, R^{a}, R^{b}, R^{c}, R^{g}, L¹, Q² being as herein defined; and that comprises reacting a different compound of formula (I) wherein Y represents an oxygen atom, L² represents an oxygen atom, a NR^{g} group and A, W, Q¹, p, R^{a}, R^{b}, R^{c}, R^{g}, L¹, Q² being as herein defined;

with a thiocarbonylation agent such as 2,4-bis(4-methoxyphenyl)-1,3,2,4-dithiadiphosphetane 2,4-disulfide, phosphorus pentasulfide, sulphur.

According to a further aspect of the present invention, there is provided a process P11 for the preparation of a compound of formula (I) wherein Y represents a NR^{d} group or an oxygen atom, L² represents a NR^{g} group and R^{g} represents a hydrogen atom, a nitro group, a cyano group, a hydroxy group, an amino group, a formyloxy group, a formylamino group, (hydroxyimino)-C₁-C₆-alkyl group, C₁-C₈-alkyl, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, or a 4-, 5-, 6- or 7-membered heterocycle comprising up to 4 heteroatoms selected in the list consisting of N, O, S; A, W, Q¹, p, R^{a}, R^{b}, R^{c}, R^{d}, L¹, Q² being as herein defined;

and that comprises reacting a different compound of formula (I) wherein Y represents a NR^{d} group or an oxygen atom, L² represents an oxygen atom, a sulphur atom; Q² represents a formyl group, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₂-C₈-halogenoalkenyl having 1 to 5 halogen atoms, C₃-C₈-halogenoalkynyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms and A, W, Q¹, p, R^{a}, R^{b}, R^{c}, R^{d}, L¹ being as herein defined;
with a compound of formula R^{g}NH wherein R^{g} being as herein defined;
optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyld iimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladiu m(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; in one or a different-pot conditions.

According to the present invention, the compounds of formula (I) useful as starting material within the processes P10 to P11 can be prepared according to process P1 to P9 according to the invention.

Thus according to a further aspect of the present invention, there is provided a process P12 for the preparation of a compound of formula (I) as illustrated by the following reaction scheme:

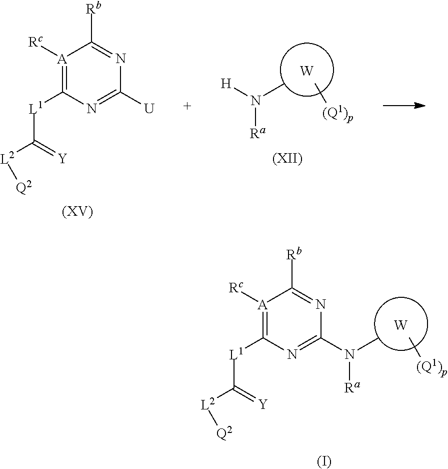

wherein
A, W, Q¹, p, R^{a}, R^{b}, R^{c}, L¹, Y, L², Q² being as herein-defined;
U represents a hydrogen atom or a leaving group such as a halogen atom, a C₁-C₆ alkylsulphenyl, a C₁-C₆ haloalkylsulphenyl; a substituted or non-substitued phenylsulphenyl, a C₁-C₆ alkylsulfonate, a C₁-C₆ haloalkylsulfonate; a substituted or non-substitued phenylsulfonate and that comprises
   reacting a compound of formula (XV) with an amino derivative of formula (XII) in order to yield a compound of formula (I), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine, optionally in the presence of an organo-metallic reagent such as an organo-lithium reagent, for example n-butyl lithium, methyl lithium, phenyl lithium or an organo-magnesium halide reagent (Grignard reagent) such as isopropyl magnesium halide more preferably such as isopropyl magnesium chloride, optionally in the presence of a base, such as an inorganic or an organic base, preferably an alkaline earth metal or alkali metal hydrides, hydroxides, amides, alcoholates, acetates, carbonates or hydrogen carbonates, such as sodium hydride, sodium amide, lithiium diisopropylamide, 2,2,6,6-tetramethylpiperidylmagnesium chloride, lithium hexamethyldisilazide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of a metallic salt such as an alkaline earth metal salt, an alkali metal salt, a transition metal salt such as a lithium salt, preferably a lithium halide, more preferably lithium chloride, such as a copper salt, preferably a copper(I) salt such as copper(I) chloride, copper(I) cyanide, in the presence of an oxidative agent such as oxygen, 3,3′,5,5′-tetra-tert-butyldiphenoquinone, 2,3-dichloro-5,6-dicyano-1,4-benzoquinone (DDQ), and 2,3,5,6-tetrachloro-1,4-benzoquinone (chloranil), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

Suitable solvents for carrying out process P1 to P12 according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulphoxides, such as dimethyl sulphoxide, or sulphones, such as sulpholane.

When carrying out process P1 to P12 according to the invention, the reaction temperatures can independently be varied within a relatively wide range. Generally, processes according to the invention are carried out at temperatures between −80° C. and 250° C.

Process P1 to P12 according to the invention is generally independently carried out under atmospheric pressure. However, in each case, it is also possible to operate under elevated or reduced pressure.

Work-up is carried out by customary methods. Generally, the reaction mixture is treated with water and the organic phase is separated off and, after drying, concentrated under reduced pressure. If appropriate, the remaining residue can be freed by customary methods, such as chromatography or recrystallization, from any impurities that may still be present.

Compounds according to the invention can be prepared according to the above described process. It will nevertheless be understood that, on the basis of his general knowledge and of available publications, the skilled worker will be able to adapt these processes according to the specifics of each of the compounds according to the invention that is desired to be synthesized.

Still in a further aspect, the present invention relates to compounds of formula (V) useful as to intermediate compounds or materials for the process of preparation according to the invention. The present invention thus provides compounds of formula (V)

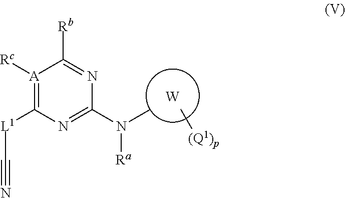

wherein A, W, Q¹, p, R^{a}, R^{b}, R^{c}, L¹ are as herein-defined.

Still in a further aspect, the present invention relates to compounds of formula (VI) useful as intermediate compounds or materials for the process of preparation according to the invention. The present invention thus provides compounds of formula (VI)

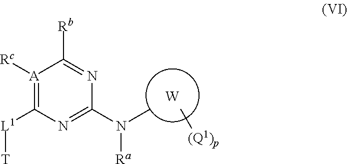

wherein W, A, Q¹, p, R^{a}, R^{b}, R^{c}, L¹, and T are as herein-defined, provided that the following compounds are excluded:
N-(3-chlorophenyl)-4-(2-chloropyridin-4-yl)-1,3,5-triazin-2-amine
4-(2-chloropyridin-4-yl)-N-[3-(1, 1,2,2-tetrafluoroethoxy)phenyl]-1,3,5-triazin-2-amine
4-(2-chloropyridin-4-yl)-N-(3,4,5-trimethoxyphenyl)-1,3,5-triazin-2-amine

Preferred compounds of formula (VI) according to the invention are those wherein T is a chlorine atom.

More preferred compounds of formula (VI) according to the invention are those selected from the group constituted of 4-(2-chloropyridin-4-yl)-N-(pyridin-3-yl)pyrimidin-2-amine, 4-(2-chloropyridin-4-yl)-N-(6-methoxypyridin-3-yl)-1,3,5-triazin-2-amine, 4-(2-chloropyridin-4-yl)-N-(3,4,5-trimethoxyphenyl)-1,3,5-triazin-2-amine, 3-{[4-(2-chloropyridin-4-yl)-1,3,5-triazin-2-yl]amino}benzoic acid, 4-(2-chloropyridin-4-yl)-N-(6-chloropyridin-2-yl)pyrimidin-2-amine, N,4-bis(2-chloropyridin-4-yl)pyrimidin-2-amine, N⁴-[4-(2-chloropyridin-4-yl)pyrimidin-2-yl]-N²-(1-methoxybutan-2-yl)pyridine-2,4-diamine, N-(3-chloro-4-fluorophenyl)-4-(2-chloropyridin-4-yl)-1,3,5-triazin-2-amine, 4-(2-chloropyridin-4-yl)-N-[3-(methylsulfanyl)phenyl]-1,3,5-triazin-2-amine, N-(3-chloro-4-methylphenyl)-4-(2-chloropyridin-4-yl)-1,3,5-triazin-2-amine, N⁴-[4-(2-chloropyridin-4-yl)pyrimidin-2-yl]-N²-cyclobutylpyridine-2,4-diamine, N⁴-[4-(2-chloropyridin-4-yl)pyrimidin-2-yl]-N²-(3-methylbutan-2-yl)pyridine-2,4-diamine, N⁴-[4-(2-chloropyridin-4-yl)pyrimidin-2-yl]-N²-(pentan-3-yl)pyridine-2,4-diamine, 4-(2-chloropyridin-4-yl)-N-(pyridin-4-yl)-1,3,5-triazin-2-amine, methyl 3-{[4-(2-chloropyridin-4-yl)-1,3,5-triazin-2-yl}amino]thiophene-2-carboxylate, ethyl 2-{[4-(2-chloropyridin-4-yl)-1,3,5-triazin-2-yl]amino}-4-methyl-1,3-thiazole-5-carboxylate, 4-(2-chloropyridin-4-yl)-N-(4-methyl-1,3-thiazol-2-yl)-1,3,5-triazin-2-amine, 4-(2-chloropyridin-4-yl)-N-(5-methyl-1,3-thiazol-2-yl)-1,3,5-triazin-2-amine, 4-(2-chloropyridin-4-yl)-N-(2-methylpyridin-4-yl)pyrimidin-2-amine, N-(2-bromopyridin-4-yl)-4-(2-chloropyridin-4-yl)pyrim din-2-amine, N-(5-bromopyridin-3-yl)-4-(2-chloropyridin-4-yl)pyrimidin-2-amine, 4-(2-chloropyridin-4-yl)-N[2-(trifluoromethyl)pyridin-4-yl]pyrimidin-2-amine, 2-{[4-(2-chloropyridin-4-yl)-1,3,5-triazin-2-yl]amino}thiophene-3-carbonitrile, N-(5-chloro-3-methylpyridin-2-yl)-4-(2-chloropyridin-4-yl)-1,3,5-triazin-2-amine, 4-(2-chloropyridin-4-yl)-N-(4-chloropyridin-3-yl)-1,3,5-triazin-2-amine, 4-(2-chloropyridin-4-yl)-N-(2-methylpyridin-4-yl)-1,3,5-triazin-2-amine, N,4-bis(2-chloropyridin-4-yl)-N-(methoxymethyl)pyrimidin-2-amine, 4-(2-chloropyridin-4-yl)-N-(2,5-difluorophenyl)-1,3,5-triazin-2-amine, 4-(2-chloropyridin-4-yl)-N-(3-fluorophenyl)-1,3,5-triazin-2-amine, 4-(2-chloropyridin-4-yl)-N-[3-(methoxymethyl)phenyl]-1,3,5-triazin-2-amine, 4-(2-chloropyridin-4-yl)-N-(thiophen-3-yl)-1,3,5-triazin-2-amine, 4-(2-chloropyridin-4-yl)-N-[3-(trifluoromethyl)phenyl]-1,3,5-triazin-2-amine, 4-(2-chloropyridin-4-yl)-N-[3-(propan-2-yl)phenyl]-1,3,5-triazin-2-amine, 4-(2-chloropyridin-4-yl)-N-[3-(1,1,2,2-tetrafluoroethoxy)phenyl]-1,3,5-triazin-2-amine, 4-(2-chloropyridin-4-yl)-N-[3-(trifluoromethoxy)phenyl]-1,3,5-triazin-2-amine, 4-(2-chloropyridin-4-yl)-N-[3-(pentafluoro-lambda⁶-sulfanyl)phenyl]-1,3,5-triazin-2-amine, 4-(2-chloropyridin-4-yl)-N-(3-ethoxyphenyl)-1,3,5-triazin-2-amine, 4-(2-chloropyridin-4-yl)-N-(3-methoxyphenyl)-1,3,5-triazin-2-amine, 4-(2-chloropyridin-4-yl)-N-phenyl-1,3,5-triazin-2-amine, 4-(2-chloropyridin-4-yl)-N-(4-fluorophenyl)-1,3,5-triazin-2-amine, 4-(2-chloropyridin-4-yl)-N-[3-(difluoromethoxy)phenyl]-1,3,5-triazin-2-amine, N-(3-{[4-(2-chloropyridin-4-yl)-1,3,5-triazin-2-yl]amino}phenyl)acetamide, and 4-(2-chloropyridin-4-yl)-N-[3-(difluoromethyl)phenyl]-1,3,5-triazin-2-amine, 4-(2-chloropyridin-4-yl)-N-[3-(difluoromethyl)-4-fluorophenyl]-1,3,5-triazin-2-amine and N-[4-chloro-3-(difluoromethyl)phenyl]-4-(2-chloropyridin-4-yl)-1,3,5-triazin-2-amine

Compounds of formula (VI) useful as intermediates for the process of preparation of compounds to of formula (I) or formula (V) may be prepared by various processes. Accordingly, there is provided a process A according to the invention for the preparation of a compound of formula (VI) wherein
R^{a} represents a hydrogen atom;
A, W, Q¹, p, R^{b}, R^{c}, L¹, T being as herein-defined; and comprising
a first step according to reaction scheme A-1:

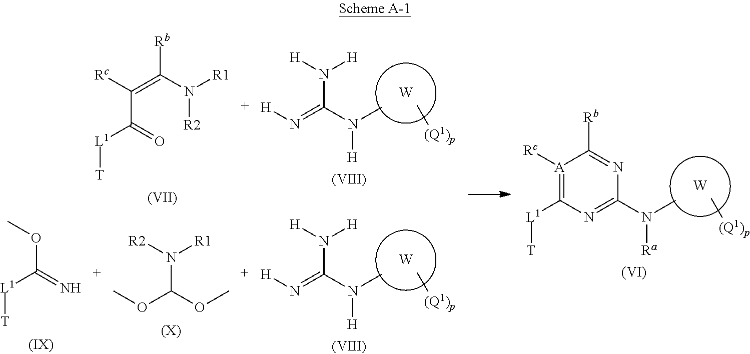

wherein
A, W, Q¹, p, R^{a}, R^{b}, R^{c}, L¹, T being as herein-defined;
R¹ and R² are independently a C₁-C₈-alkyl group, R¹ and R² can form together a, 4-, 5-, 6- or 7-membered heterocycle comprising up to 4 heteroatoms selected in the list consisting of N, O, S;
   that comprises the formation of the pyrimidine or triazine moiety by condensation, at a temperature of from −50° C. to 200° C., of a compound of formula (VII) or formula (IX), this in presence a compound of formula (X), optionally in the presence of a base such as an inorganic or an organic base, preferably an alkaline earth metal or alkali metal hydrides, hydroxides, amides, alcoholates, acetates, carbonates or hydrogen carbonates, such as sodium hydride, sodium amide, lithiium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); with a guanidine or a guanidine salt derivative of formula (VIII) to yield a compound of formula (VI).

Alternatively, there is provided a process B according to the invention for the preparation of a compound of formula (VI) wherein A, W, Q¹, p, R^{a}, R^{b}, R^{c}, L¹, T being as herein-defined; and comprising
a first step according to reaction scheme B-1:

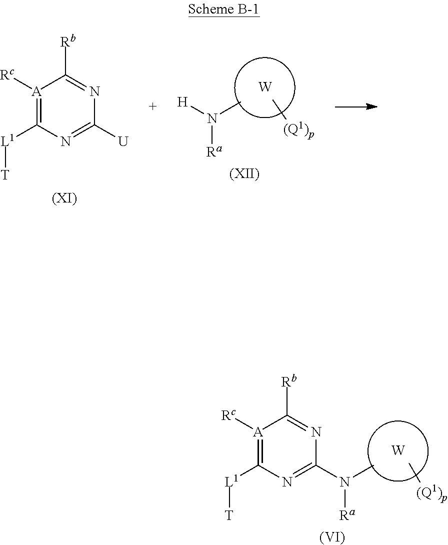

wherein
A, W, Q¹, p, R^{a}, R^{b}, R^{c}, L¹, T being as herein-defined;
U represents a hydrogen atom or a leaving group such as a halogen atom, a C₁-C₆ alkylsulphenyl, a C₁-C₆ haloalkylsulphenyl; a substituted or non-substitued phenylsulphenyl, a C₁-C₆ alkylsulfonate, a C₁-C₆ haloalkylsulfonate; a substituted or non-substitued phenylsulfonate and that comprises
   reacting a compound of formula (XI) with an amino derivative of formula (XII) in order to yield a compound of formula (VI), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, to bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(+1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine, optionally in the presence of an organo-metallic reagent such as an organo-lithium reagent, for example n-butyl lithium, methyl lithium, phenyl lithium or an organo-magnesium halide reagent (Grignard reagent) such as isopropyl magnesium halide more preferably such as isopropyl magnesium chloride, optionally in the presence of a base, such as an inorganic or an organic base, preferably an alkaline earth metal or alkali metal hydrides, hydroxides, amides, alcoholates, acetates, carbonates or hydrogen carbonates, such as sodium hydride, sodium amide, lithiium diisopropylamide, 2,2,6,6-tetramethylpiperidylmagnesium chloride, lithium hexamethyldisilazide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of a metallic salt such as an alkaline earth metal salt, an alkali metal salt, a transition metal salt such as a lithium salt, preferably a lithium halide, more preferably lithium chloride, such as a copper salt, preferably a copper(I) salt such as copper(I) chloride, copper(I) cyanide, in the presence of an oxidative agent such as oxygen, 3,3′,5,5′-tetra-tert-butyldiphenoquinone, 2,3-dichloro-5,6-dicyano-1,4-benzoquinone (DDQ), and 2,3,5,6-tetrachloro-1,4-benzoquinone (chloranil), optionally in the presence of an acid such as a Lewis acid; notably metal or metalloïd halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

Alternatively, there is provided a process C according to the invention for the preparation of a compound of formula (VI) wherein A, W, Q¹, p, R^{a}, R^{b}, R^{c}, L¹, T, being as herein-defined; and comprising
a first step according to reaction scheme C-1:

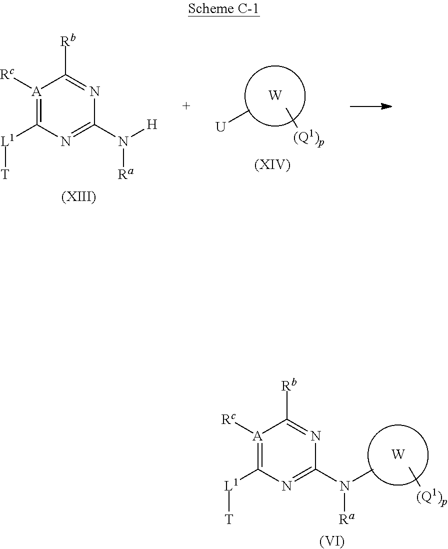

wherein
A, W, Q¹, p, R^{a}, R^{b}, R^{c}, L¹, T, being as herein-defined;
U represents a leaving group such as a halogen atom, a C₁-C₆ alkylsulphenyl, a C₁-C₆ haloalkylsulphenyl; a substituted or non-substitued phenylsulphenyl a C₁-C₆ alkylsulfonate, a C₁-C₆ haloalkylsulfonate; a substituted or non-substitued phenylsulfonate, and that comprises
   reacting an amino derivative of formula (XIII) with a compound of formula (XIV) in order to yield a compound of formula (VI), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(+1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine, optionally in the presence of an organo-metallic reagent such as an organo-lithium reagent for example n-butyl lithium, methyl lithium, phenyl lithium or an organo-magnesium halide reagent (Grignard reagent) such as isopropyl magnesium halide for example isopropyl magnesium chloride, optionally in the presence of a base, such as an inorganic or an organic base; preferably an alkaline earth metal or alkali metal hydrides, hydroxides, amides, alcoholates, acetates, carbonates or hydrogen carbonates, such as sodium hydride, sodium amide, lithiium diisopropylamide, 2,2,6,6-tetramethylpiperidylmagnesium chloride, lithium hexamethyldisilazide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of a metallic salt such as an alkaline earth metal salt, an alkali metal salt, a transition metal salt such as a lithium salt, preferably a lithium halide, more preferably lithium chloride, such as a copper salt, preferably a copper(I) salt such as copper(I) chloride, copper(I) cyanide, in the presence of an oxidative agent such as oxygen, 3,3′,5,5′-tetra-tert-butyldiphenoquinone, 2,3-dichloro-5,6-dicyano-1,4-benzoquinone (DDQ), and 2,3,5,6-tetrachloro-1,4-benzoquinone (chloranil).

In a further aspect, the present invention also relates to a fungicide composition comprising an effective and non-phytotoxic amount of an active compound of formula (I).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicide composition according to the invention. This amount can be determined by systematic field trials, which are within the capabilities of a person skilled in the art.

Thus, according to the invention, there is provided a fungicide composition comprising, as an active ingredient, an effective amount of a compound of formula (I) as herein defined and an agriculturally acceptable support, carrier or filler.

According to the invention, the term “support” denotes a natural or synthetic, organic or inorganic compound with which the active compound of formula (I) is combined or associated to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition according to the invention may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active compound and/or the inert support are water-insoluble and when the vector agent for the application is water. Preferably, surfactant content may be comprised from 5% to 40% by weight of the composition.

Optionally, additional components may also be included, e.g. protective colloids, adhesives, thickeners, thixotropic agents, penetration agents, stabilisers, sequestering agents. More generally, the active compounds can be combined with any solid or liquid additive, which complies with the usual formulation techniques.

In general, the composition according to the invention may contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed to coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions which are ready to be applied to the plant or seed to be treated by means of a suitable device, such as a spraying or dusting device, but also concentrated commercial compositions which must be diluted before application to the crop.

The compounds according to the invention can also be mixed with one or more insecticide, fungicide, bactericide, attractant, acaricide or pheromone active substance or other compounds with biological activity. The mixtures thus obtained have normally a broadened spectrum of activity. The mixtures with other fungicide compounds are particularly advantageous.

Examples of suitable fungicide mixing partners may be selected in the following lists:

(1) Inhibitors of the nucleic acid synthesis, for example benalaxyl, benalaxyl-M, bupirimate, clozylacon, dimethirimol, ethirimol, furalaxyl, hymexazol, metalaxyl, metalaxyl-M, ofurace, oxadixyl and oxolinic acid.
(2) Inhibitors of the mitosis and cell division, for example benomyl, carbendazim, chlorfenazole, diethofencarb, ethaboxam, fuberidazole, pencycuron, thiabendazole, thiophanate, thiophanate-methyl and zoxamide.
(3) Inhibitors of the respiration, for example diflumetorim as C₁-respiration inhibitor; bixafen, boscalid, carboxin, fenfuram, flutolanil, fluopyram, furametpyr, furmecyclox, isopyrazam (mixture of syn-epimeric racemate 1RS,4SR,9RS and anti-epimeric racemate 1RS,4SR,9SR), isopyrazam (syn epimeric racemate 1R,4SR,9RS), isopyrazam (syn-epimeric enantiomer 1R,4S,9R), isopyrazam (syn-epimeric enantiomer 1S,4R,9S), isopyrazam (anti-epimeric racemate 1RS,4SR,9SR), isopyrazam (anti-epimeric enantiomer 1R,4S,9S), isopyrazam (anti-epimeric enantiomer 1S,4R,9R), mepronil, oxycarboxin, penflufen, penthiopyrad, sedaxane, thifluzamide as CII-respiration inhibitor; amisulbrom, azoxystrobin, cyazofamid, dimoxystrobin, enestroburin, famoxadone, fenamidone, fluoxastrobin, kresoxim-methyl, metominostrobin, orysastrobin, picoxystrobin, pyraclostrobin, pyraoxystrobin, pyrametostrobin, pyribencarb, trifloxystrobin as CIII-respiration inhibitor.
(4) Compounds capable to act as an uncoupler, like for example binapacryl, dinocap, fluazinam to and meptyldinocap.
(5) Inhibitors of the ATP production, for example fentin acetate, fentin chloride, fentin hydroxide, and silthiofam.
(6) Inhibitors of the amino acid and/or protein biosynthesis, for example andoprim, blasticidin-S, cyprodinil, kasugamycin, kasugamycin hydrochloride hydrate, mepanipyrim and pyrimethanil.
(7) Inhibitors of the signal transduction, for example fenpiclonil, fludioxonil and quinoxyfen.
(8) Inhibitors of the lipid and membrane synthesis, for example biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl and vinclozolin.
(9) Inhibitors of the ergosterol biosynthesis, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole and voriconazole.
(10) Inhibitors of the cell wall synthesis, for example benthiavalicarb, dimethomorph, flumorph, iprovalicarb, mandipropamid, polyoxins, polyoxorim, prothiocarb, validamycin A, and valifenalate.
(11) Inhibitors of the melanine biosynthesis, for example carpropamid, diclocymet, fenoxanil, phthalide, pyroquilon and tricyclazole.
(12) Compounds capable to induce a host defence, like for example acibenzolar-S-methyl, probenazole, and tiadinil.
(13) Compounds capable to have a multisite action, like for example bordeaux mixture, captafol, captan, chlorothalonil, copper naphthenate, copper oxide, copper oxychloride, copper preparations such as copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulphur and sulphur preparations including calcium polysulphide, thiram, tolylfluanid, zineb and ziram.
(14) Further compounds like for example 2,3-dibutyl-6-chlorothieno[2,3-d]pyrimidin-4(3H)-one, ethyl (2Z)-3-amino-2-cyano-3-phenylprop-2-enoate, N-[2-(1,3-dimethylbutyl)phenyl]-5-fluoro-1,3-dimethyl-1H-pyrazole-4-carboxamide, 3-(difluoromethyl)-1-methyl-N-(3′,4′,5′-trifluorobiphenyl-2-yl)-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, (2E)-2-(2-{[6-(3-chloro-2-methylphenoxy)-5-fluoropyrimidin-4-yl]oxy}phenyl)-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, N-(3-ethyl-3,5,5-trimethylcyclohexyl)-3-(formylamino)-2-hydroxybenzamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N-ethyl-N-methyl-N′-{2-methyl-5-(trifluoromethyl)-4-[3-(trimethylsilyl)propoxy]phenyl}imidoformamide, N′-{5-(difluoromethyl)-2-methyl-4-[3-(trimethylsily)propoxy]phenyl}-N-ethyl-N-methylimidoformamide, O-{1-[(4-methoxyphenoxy)methyl]-2,2-dimethylpropyl}1H-imidazole-1-carbothioate, N-[2-(4-{[3-(4-chlorophenyl)prop-2-yn-1-yl]oxy}-3-methoxyphenyl)ethyl]-N²-(methylsulfonyl)valinamide, 5-chloro-7-(4-methylpiperidin-1-yl)-6-(2,4,6-trifluorophenyl)[1,2,4]triazolo[1,5-a]pyrimidine, 5-amino-1,3,4-thiadiazole-2-thiol, propamocarb-fosetyl, 1-[(4-methoxyphenoxy)methyl]-2,2-dimethylpropyl 1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulfonyl)pyridine, 2-butoxy-6-iodo-3-propyl-4H-chromen-4-one, 2-phenylphenol and salts, 3-(difluoromethyl)-1-methyl-N-[2-(1,1,2,2-tetrafluoroethoxy)phenyl]-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, quinolin-8-ol, quinolin-8-ol sulfate (2:1) (salt), tebufloquin, 5-methyl-6-octyl-3,7-dihydro[1,2,4]triazolo[1,5-a]pyrimidin-7-amine, 5-ethyl-6-octyl-3,7-dihydro[1,2,4]triazolo[1,5-a]pyrimidin-7-amine, ametoctradin, benthiazole, bethoxazin, capsimycin, carvone, chinomethionat, chloroneb, cufraneb, cyflufenamid, cymoxanil, cyprosulfamide, dazomet, debacarb, dichlorophen, diclomezine, dicloran, difenzoquat, difenzoquat methylsulphate, diphenylamine, ecomate, ferimzone, flumetover, fluopicolide, fluoroimide, flusulfamide, flutianil, fosetyl-aluminium, fosetyl-calcium, fosetyl-sodium, hexachlorobenzene, irumamycin, isotianil, methasulfocarb, methyl (2E)-2-{2-[({cyclopropyl[(4-methoxyphenyl)imino]methyl}thio)methyl]phenyl}-3-methoxyacrylate, methyl isothiocyanate, metrafenone, (5-chloro-2-methoxy-4-methylpyridin-3-yl)(2,3,4-trimethoxy-6-methylphenyl)methanone, mildiomycin, tolnifanide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(Z)-[(cyclopropylmethoxy)imino][6-(difluoromethoxy)-2,3-difluorophenyl]methyl}-2-phenylacetamide, N-{(E)-[(cyclopropylmethoxy)imino][6-(difluoromethoxy)-2,3-difluorophenyl]methyl}-2-phenylacetamide, natamycin, nickel dimethyldithiocarbamate, nitrothal-isopropyl, octhilinone, oxamocarb, oxyfenthiin, pentachlorophenol and salts, phenazine-1-carboxylic acid, phenothrin, phosphorous acid and its salts, propamocarb fosetylate, propanosine-sodium, proquinazid, pyrroInitrine, quintozene, S-prop-2-en-1-yl 5-amino-2-(1-methylethyl)-4-(2-methylphenyl)-3-oxo-2,3-dihydro-1H-pyrazole-1-carbothioate, tecloftalam, tecnazene, triazoxide, trichlamide, 5-chloro-N′-phenyl-N′-prop-2-yn-1-ylthiophene-2-sulfonohydrazide, zarilamid, N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, 3-(difluoromethyl)-N-[4-fluoro-2-(1,1,2,3,3,3-hexafluoropropoxy)phenyl]-1-methyl-1H-pyrazole-4-carboxamide and pentyl {6-[({[(1-methyl-1H-tetrazol-5-yl)(phenyl)methylidene]amino}oxy)methyl]pyridin-2-yl}carbamate.

The composition according to the invention comprising a mixture of a compound of formula (I) with a bactericide compound may also be particularly advantageous. Examples of suitable bactericide mixing partners may be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The compounds of formula (I) and the fungicide composition according to the invention can be used to curatively or preventively control the phytopathogenic fungi of plants or crops.

Thus, according to a further aspect of the invention, there is provided a method for curatively or preventively controlling the phytopathogenic fungi of plants or crops characterised in that a compound of formula (I) or a fungicide composition according to the invention is applied to the seed, the plant or to the fruit of the plant or to the soil wherein the plant is growing or wherein it is desired to grow.

The method of treatment according to the invention may also be useful to treat propagation material such as tubers or rhizomes, but also seeds, seedlings or seedlings pricking out and plants or plants pricking out. This method of treatment can also be useful to treat roots. The method of treatment according to the invention can also be useful to treat the over ground parts of the plant such as trunks, stems or stalks, leaves, flowers and fruit of the concerned plant.

Among the plants that can be protected by the method according to the invention, mention may be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantins), Rubiaceae sp., Theaceae sp., Sterculiceae sp., Rutaceae sp. (for instance lemons, oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

Among the diseases of plants or crops that can be controlled by the method according to the invention, mention may be made of:
Powdery Mildew Diseases such as
   Blumeria diseases caused for example by Blumeria graminis;
   Podosphaera diseases caused for example by Podosphaera leucotricha;
   Sphaerotheca diseases caused for example by Sphaerotheca fuliginea;
   Uncinula diseases caused for example by Uncinula necator;
Rust Diseases such as
   Gymnosporangium diseases caused for example by Gymnosporangium sabinae;
   Hemileia diseases caused for example by Hemileia vastatrix;
   Phakopsora diseases caused for example by Phakopsora pachyrhizi and Phakopsora meibomiae;
   Puccinia diseases caused for example by Puccinia recondite, Puccinia graminis or Puccinia striiformis;
   Uromyces diseases caused for example by Uromyces appendiculatus;
Oomycete Diseases such as
   Albugo diseases caused for example by Albugo candida;
   Bremia diseases caused for example by Bremia lactucae;
   Peronospora diseases caused for example by Peronospora pisi and Peronospora brassicae;
   Phytophthora diseases caused for example by Phytophthora infestans;
   Plasmopara diseases caused for example by Plasmopara viticola;
   Pseudoperonospora diseases caused for example by Pseudoperonospora humuli and
   Pseudoperonospora cubensis;
   Pythium diseases caused for example by Pythium ultimum;
Leaf spot, Leaf blotch and Leaf Blight Diseases such as
   Alternaria diseases caused for example by Alternaria solani;
   Cercospora diseases caused for example by Cercospora beticola;
   Cladiosporium diseases caused for example by Cladiosporium cucumerinum;
   Cochliobolus diseases caused for example by Cochliobolus sativus (Conidiaform: Drechslera, Syn: Helminthosporium) or Cochliobolus miyabeanus;
   Colletotrichum diseases caused for example by Colletotrichum lindemuthianum;
   Cycloconium diseases caused for example by Cycloconium oleaginum;
   Diaporthe diseases caused for example by Diaporthe citri;
   Elsinoe diseases caused for example by Elsinoe fawcettii;
   Gloeosporium diseases caused for example by Gloeosporium laeticolor;
   Glomerella diseases caused for example by Glomerella cingulata;
   Guignardia diseases caused for example by Guignardia bidwellii;
   Leptosphaeria diseases caused for example by Leptosphaeria maculans and Leptosphaeria nodorum;
   Magnaporthe diseases caused for example by Magnaporthe grisea;
   Mycosphaerella diseases caused for example by Mycosphaerella graminicola,
   Mycosphaerella arachidicola and Mycosphaerella fijiensis;
   Phaeosphaeria diseases caused for example by Phaeosphaeria nodorum;
   Pyrenophora diseases caused for example by Pyrenophora teres or Pyrenophora tritici repentis;
   Ramularia-diseases caused for example by Ramularia collo-cygni or Ramularia areola;
   Rhynchosporium diseases caused for example by Rhynchosporium secalis;
   Septoria diseases caused for example by Septoria apii and Septoria lycopersici;
   Typhula diseases caused for example by Thyphula incarnate;
   Venturia diseases caused for example by Venturia inaequalis;
Root-, Sheath and Stem Diseases such as
   Corticium diseases caused for example by Corticium graminearum;
   Fusarium diseases caused for example by Fusarium oxysporum;
   Gaeumannomyces diseases caused for example by Gaeumannomyces graminis;
   Rhizoctonia diseases caused for example by Rhizoctonia solani;
   Sarocladium diseases caused for example by Sarocladium oryzae;
   Sclerotium diseases caused for example by Sclerotium oryzae;
   Tapesia diseases caused for example by Tapesia acuformis;
   Thielaviopsis diseases caused for example by Thielaviopsis basicola;
Ear and Panicle Diseases including Maize cob such as
   Alternaria diseases caused for example by Alternaria spp.;
   Aspergillus diseases caused for example by Aspergillus flavus;
   Cladosporium diseases caused for example by Cladiosporium cladosporioides;
   Claviceps diseases caused for example by Claviceps purpurea;
   Fusarium diseases caused for example by Fusarium culmorum;
   Gibberella diseases caused for example by Gibberella zeae;
   Monographella diseases caused for example by Monographella nivalis;
Smut- and Bunt Diseases such as
   Sphacelotheca diseases caused for example by Sphacelotheca reiliana;
   Tilletia diseases caused for example by Tilletia caries;
   Urocystis diseases caused for example by Urocystis occulta;
   Ustilago diseases caused for example by Ustilago nuda;
Fruit Rot and Mould Diseases such as
   Aspergillus diseases caused for example by Aspergillus flavus;
   Botrytis diseases caused for example by Botrytis cinerea;
   Penicillium diseases caused for example by Penicillium expansum and Penicillium purpurogenum;
   Rhizopus diseases caused by example by Rhizopus stolonifer
   Sclerotinia diseases caused for example by Sclerotinia sclerotiorum;
   Verticillium diseases caused for example by Verticillium alboatrum;
Seed- and Soilborne Decay, Mould, Wilt, Rot and Damping-off diseases
   Alternaria diseases caused for example by Alternaria brassicicola;
   Aphanomyces diseases caused for example by Aphanomyces euteiches;
   Ascochyta diseases caused for example by Ascochyta lentis;
   Aspergillus diseases caused for example by Aspergillus flavus;
   Cladosporium diseases caused for example by Cladosporium herbarum;
   Cochliobolus diseases caused for example by Cochliobolus sativus;
   (Conidiaform: Drechslera, Bipolaris Syn: Helminthosporium);
   Colletotrichum diseases caused for example by Colletotrichum coccodes;
   Fusarium diseases caused for example by Fusarium culmorum;
   Gibberella diseases caused for example by Gibberella zeae;
   Macrophomina diseases caused for example by Macrophomina phaseolina;
   Microdochium diseases caused for example by Microdochium nivale;
   Monographella diseases caused for example by Monographella nivalis;
   Penicillium diseases caused for example by Penicillium expansum;
   Phoma diseases caused for example by Phoma lingam;
   Phomopsis diseases caused for example by Phomopsis sojae;
   Phytophthora diseases caused for example by Phytophthora cactorum;
   Pyrenophora diseases caused for example by Pyrenophora graminea;
   Pyricularia diseases caused for example by Pyricularia oryzae;
   Pythium diseases caused for example by Pythium ultimum;
   Rhizoctonia diseases caused for example by Rhizoctonia solani;
   Rhizopus diseases caused for example by Rhizopus oryzae;
   Sclerotium diseases caused for example by Sclerotium rolfsii;
   Septoria diseases caused for example by Septoria nodorum;
   Typhula diseases caused for example by Typhula incarnate;
   Verticillium diseases caused for example by Verticillium dahliae;
Canker, Broom and Dieback Diseases such as
   Nectria diseases caused for example by Nectria galligena;
Blight Diseases such as
   Monilinia diseases caused for example by Monilinia laxa;
Leaf Blister or Leaf Curl Diseases including deformation of blooms and fruits such as
   Exobasidium diseases caused for example by Exobasidium vexans.
   Taphrina diseases caused for example by Taphrina deformans;
Decline Diseases of Wooden Plants such as
   Esca disease caused for example by Phaeomoniella clamydospora, Phaeoacremonium aleophilum and Fomitiporia mediterranea;
   Ganoderma diseases caused for example by Ganoderma boninense;
   Rigidoporus diseases caused for example by Rigidoporus lignosus
Diseases of Flowers and Seeds such as
   Botrytis diseases caused for example by Botrytis cinerea;
Diseases of Tubers such as
   Rhizoctonia diseases caused for example by Rhizoctonia solani;
   Helminthosporium diseases caused for example by Helminthosporium solani;
Club root diseases such as
   Plasmodiophora diseases, cause for example by Plamodiophora brassicae.
Diseases caused by Bacterial Organisms such as
   Xanthomonas species for example Xanthomonas campestris pv. oryzae;
   Pseudomonas species for example Pseudomonas syringae pv. lachrymans;
   Erwinia species for example Erwinia amylovora.

The fungicide composition according to the invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds according to the invention, or a composition according to the invention; this includes for example direct application, spraying, dipping, injection or any other suitable means.

The dose of active compound usually applied in the method of treatment according to the to invention is generally and advantageously from 10 to 800 g/ha, preferably from 50 to 300 g/ha for applications in foliar treatment. The dose of active substance applied is generally and advantageously from 2 to 200 g per 100 kg of seed, preferably from 3 to 150 g per 100 kg of seed in the case of seed treatment.

It is clearly understood that the doses indicated herein are given as illustrative examples of the method according to the invention. A person skilled in the art will know how to adapt the application doses, notably according to the nature of the plant or crop to be treated.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, co suppression technology or RNA interference—RNAi-technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are also suitable for mobilizing the defense system of the plant against attack by unwanted phytopathogenic fungi and/or microorganisms and/or viruses. This may, if appropriate, be one of the reasons of the enhanced activity of the combinations according to the invention, for example against fungi. Plant-strengthening (resistance-inducing) substances are to be understood as meaning, in the present context, those substances or combinations of substances which are capable of stimulating the defense system of plants in such a way that, when subsequently inoculated with unwanted phytopathogenic fungi and/or microorganisms and/or viruses, the treated plants display a substantial degree of resistance to these unwanted phytopathogenic fungi and/or microorganisms and/or viruses. In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozon exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced to seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species (WO 1992/005251, WO 1995/009910, WO 1998/27806, WO 2005/002324, WO 2006/021972 and U.S. Pat. No. 6,229,072). However, genetic determinants for male sterility can also be located in the nuclear genome. Male sterile plants can also be obtained by plant biotechnology methods such as genetic engineering. A particularly useful means of obtaining male-sterile plants is described in WO 1989/10396 in which, for example, a ribonuclease such as barnase is selectively expressed in the tapetum cells in the stamens. Fertility can then be restored by expression in the tapetum cells of a ribonuclease inhibitor such as barstar (e.g. WO 1991/002069).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a Petunia EPSPS (Shah et al., Science (1986), 233, 478-481), a Tomato EPSPS (Gasser et al., J. Biol. Chem. (1988), 263, 4280-4289), or an Eleusine EPSPS (WO 2001/66704). It can also be a mutated EPSPS as described in for example EP-A 0837944, WO 2000/066746, WO 2000/066747 or WO 2002/026995. Glyphosate-tolerant plants can also be obtained by expressing a gene that encodes a glyphosate oxido-reductase enzyme as described in U.S. Pat. No. 5,776,760 and U.S. Pat. No. 5,463,175. Glyphosate-tolerant plants can also be obtained by expressing a gene that encodes a glyphosate acetyl transferase enzyme as described in for example WO 2002/036782, WO 2003/092360, WO 2005/012515 and WO 2007/024782. Glyphosate-tolerant plants can also be obtained by selecting plants containing naturally-occurring mutations of the above-mentioned genes, as described in for example WO 2001/024615 or WO 2003/013226. Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665. Further herbicide-tolerant plants are also plants that are made tolerant to the herbicides inhibiting the enzyme hydroxyphenylpyruvated ioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme as described in WO 1996/038567, WO 1999/024585 and WO 1999/024586. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 1999/034008 and WO 2002/36787. Tolerance of plants to HPPD inhibitors can also be improved by transforming plants with a gene encoding an enzyme prephenate dehydrogenase in addition to a gene encoding an HPPD-tolerant enzyme, as described in WO 2004/024928.

Still further herbicide resistant plants are plants that are made tolerant to acetolactate synthase

(ALS) inhibitors. Known ALS-inhibitors include, for example, sulfonylurea, imidazolinone, triazolopyrimidines, pyrimidinyloxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright, Weed Science (2002), 50, 700-712, but also, in U.S. Pat. No. 5,605,011, U.S. Pat. No. 5,378,824, U.S. Pat. No. 5,141,870, and U.S. Pat. No. 5,013,659. The production of sulfonylurea-tolerant plants and imidazolinone-tolerant plants is described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and international publication WO 1996/033270. Other imidazolinone-tolerant plants are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerant plants are also described in for example WO 2007/024782.

Other plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 1997/41218, for sugar beet in U.S. Pat. No. 5,773,702 and WO 1999/057965, for lettuce in U.S. Pat. No. 5,198,599, or for sunflower in WO 2001/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance. An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed by Crickmore et al., Microbiology and Molecular Biology Reviews (1998), 62, 807-813, updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1F, Cry2Ab, Cry3Aa, or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al., Nat. Biotechnol. (2001), 19, 668-72; Schnepf et al., Applied Environm. Microbiol. (2006), 71, 1765-1774); or
3) a hybrid insecticidal protein comprising parts of different insecticidal crystal proteins from Bacillus thuringiensis, such as a hybrid of the proteins of 1) above or a hybrid of the proteins of 2) above, e.g., the CryIA.105 protein produced by corn event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604;
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 1994/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102.

Of course, an insect-resistant transgenic plant, as used herein, also includes any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected when using different proteins directed at different target insect species, or to delay insect resistance development to the plants by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of poly(ADP-ribose)polymerase (PARP) gene in the plant cells or plants as described in WO 2000/004173 or WO2006/045633 or PCT/EP07/004,142.
b. plants which contain a stress tolerance enhancing transgene capable of reducing the expression and/or the activity of the PARG encoding genes of the plants or plants cells, as described e.g. in WO 2004/090140.
c. plants which contain a stress tolerance enhancing transgene coding for a plant-functional enzyme of the nicotinamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphoribosyltransferase as described e.g. in WO2006/032469 or WO 2006/133827 or PCT/EP07/002,433.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed, for example, in EP 0571427, WO 1995/004826, EP 0719338, WO 1996/15248, WO 1996/19581, WO 1996/27674, WO 1997/11188, WO 1997/26362, WO 1997/32985, WO 1997/42328, WO 1997/44472, WO 1997/45545, WO 1998/27212, WO 1998/40503, WO99/58688, WO 1999/58690, WO 1999/58654, WO 2000/008184, WO 2000/008185, WO 2000/008175, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 1995/26407, WO 1996/34968, WO 1998/20145, WO 1999/12950, WO 1999/66050, WO 1999/53072, U.S. Pat. No. 6,734,341, WO 2000/11192, WO 1998/22604, WO 1998/32326, WO 2001/98509, WO 2001/98509, WO 2005/002359, U.S. Pat. No. 5,824,790, U.S. Pat. No. 6,013,861, WO 1994/004693, WO 1994/009144, WO 1994/11520, WO 1995/35026, WO 1997/20936.
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, as disclosed in EP 0663956, WO 1996/001904, WO 1996/021023, WO 1998/039460, and WO 1999/024593, plants producing alpha 1,4 glucans as disclosed in WO 1995/031553, US 2002/031826, U.S. Pat. No. 6,284,479, U.S. Pat. No. 5,712,107, WO 1997/047806, WO 1997/047807, WO 1997/047808 and WO 2000/014249, plants producing alpha-1,6 branched alpha-1,4-glucans, as disclosed in WO 2000/73422, plants producing alternan, as disclosed in WO 2000/047727, EP 06077301.7, U.S. Pat. No. 5,908,975 and EP 0728213,
3) transgenic plants which produce hyaluronan, as for example disclosed in WO 2006/032538, WO 2007/039314, WO 2007/039315, WO 2007/039316, JP 2006/304779, and WO 2005/012529.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes as described in WO 1998/000549
b) Plants, such as cotton plants, containing an altered form of rsw2 or rsw3 homologous nucleic acids as described in WO2004/053219
c) Plants, such as cotton plants, with increased expression of sucrose phosphate synthase as described in WO 2001/017333
d) Plants, such as cotton plants, with increased expression of sucrose synthase as described in WO02/45485
e) Plants, such as cotton plants, wherein the timing of the plasmodesmatal gating at the basis of the fiber cell is altered, e.g. through downregulation of fiberselective β 1,3-glucanase as described in WO2005/017157
f) Plants, such as cotton plants, having fibers with altered reactivity, e.g. through the expression of N-acteylglucosaminetransferase gene including nodC and chitinsynthase genes as described in WO2006/136351

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants contain a mutation imparting such altered oil characteristics and include:
a) Plants, such as oilseed rape plants, producing oil having a high oleic acid content as described e.g. in U.S. Pat. No. 5,969,169, U.S. Pat. No. 5,840,946 or U.S. Pat. No. 6,323,392 or U.S. Pat. No. 6,063,947
b) Plants such as oilseed rape plants, producing oil having a low linolenic acid content as described in U.S. Pat. No. 6,270,828, U.S. Pat. No. 6,169,190 or U.S. Pat. No. 5,965,755
c) Plant such as oilseed rape plants, producing oil having a low level of saturated fatty acids as described e.g. in U.S. Pat. No. 5,434,283

Particularly useful transgenic plants which may be treated according to the invention are plants which comprise one or more genes which encode one or more toxins, such as the following which are sold under the trade names YIELD GARD₃ (for example maize, cotton, soya beans), KnockOut₃ (for example maize), BiteGard₃ (for example maize), Bt-Xtra₃ (for example maize), StarLink₃ (for example maize), Bollgard₃ (cotton), Nucotn₃ (cotton), Nucotn 33B® (cotton), NatureGard₃ (for example maize), Protecta₃ and NewLeaf₃ (potato). Examples of herbicide-tolerant plants which may be mentioned are maize varieties, cotton varieties and soya bean varieties which are sold under the trade names Roundup Ready₃ (tolerance to glyphosate, for example maize, cotton, soya bean), Liberty Link₃ (tolerance to phosphinotricin, for example oilseed rape), IMI₃ (tolerance to imidazolinones) and STS₃ (tolerance to sulphonylureas, for example maize). Herbicide-resistant plants (plants bred in a conventional manner for herbicide tolerance) which may be mentioned include the varieties sold under the name Clearfield₃ (for example maize).

Particularly useful transgenic plants which may be treated according to the invention are plants containing transformation events, or combination of transformation events, that are listed for example in the databases from various national or regional regulatory agencies (see for example http://dmoinfo.irc.it/gmp_browse.aspx and http://www.agbios.com/dbase.php).

The compounds or mixtures according to the invention may also be used for the preparation of composition useful to curatively or preventively treat human or animal fungal diseases such as, for example, mycoses, dermatoses, trichophyton diseases and candidiases or diseases caused by Aspergillus spp., for example Aspergillus fumigatus.

Furthermore compounds according to the invention may also be used to reduce the contents of mycotoxins in plants and the harvested plant material and therefore in foods and animal feed stuff made therefrom.

Method of combating phytopathogenic and mycotoxin producing fungi characterized in that compounds according to the invention are applied to these fungi and/or their habitat.

Especially but not exclusively the following mycotoxins can be specified:

Deoxynivalenole (DON), Nivalenole, 15-Ac-DON, 3-Ac-DON, T2- and HT2-Toxins, Fumonisines, Zearalenone Moniliformine, Fusarine, Diaceotoxyscirpenole (DAS), Beauvericine, Enniatine, Fusaroproliferine, Fusarenole, Ochratoxines, Patuline, Ergotalkaloides and Aflatoxines, which are caused for example by the following fungal diseases: Fusarium spec., like Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides and others but also by Aspergillus spec., Penicillium spec., Claviceps purpurea, Stachybotrys spec. and others.

The various aspects of the invention will now be illustrated with reference to the following tables I, II and III of compound examples and the following preparation or efficacy examples.

The following tables I, II and III illustrate in a non-limiting manner examples of compounds according to the invention.

In the following tables, M+H (or M−H) means the molecular ion peak, plus or minus 1 a.m.u. (atomic mass unit) respectively, as observed in mass spectroscopy and M (Apcl+) means the molecular ion peak as it was found via positive atmospheric pressure chemical ionisation in mass spectroscopy.

TABLE I
Formula (III₂)

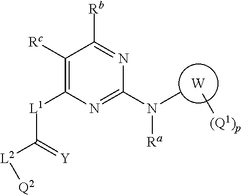

| | | | | | In Table I we use the following abbreviations for | | | | | | | | |
|---|---|---|---|---|---|---|---|---|---|---|---|---|---|
| | | | | | *—L¹—C(═Y)—L²—Q²: | | | | | | | | |
| 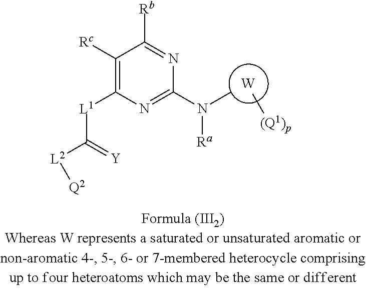 | | | | | *—L¹—C(═Y)—L²—Q² 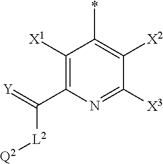 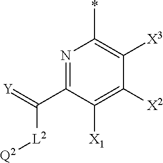 | | | | | Abbreviation L1A L1B | | | |
| | | | | | *-L¹—C(═Y)—L²—Q² | | | | | | | | |
| Ex- ample Num- ber | L¹C(═Y)L²Q² | L² | Q² | Y | 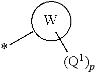 | X¹ | X² | X³ | R^{a} | R^{b} | R^{c} | Mea- sured MW | log p |
| A1 | L1A | oxy | ethyl | oxo | 6-methoxypyridin- | H | H | H | H | H | H | 351 | 1.1 |
| | | | | | 3-yl | | | | | | | | |
| A2 | L1A | bond | 3,6-dihydro- | oxo | 2-chloropyridin- | H | H | H | H | H | H | 393 | 2.2 |
| | | | pyridin-1(2H)-yl | | 4-yl | | | | | | | | |
| A3 | L1A | bond | 3,6-dihydro- | oxo | 2-(3,6- | H | H | H | H | H | H | 468 | 1.6 |
| | | | pyridin- | | dihydropyridin- | | | | | | | | |
| | | | 1(2H)-yl | | 1(2H)-ylcarbonyl) | | | | | | | | |
| | | | | | pyridin-4-yl | | | | | | | | |
| A4 | L1A | methylimino | 2-methoxy-ethyl | oxo | 2-[(2- | H | H | H | H | H | H | 480 | 1.1 |
| | | | | | methoxyethyl) | | | | | | | | |
| | | | | | (methyl) | | | | | | | | |
| | | | | | carbamoyl] | | | | | | | | |
| | | | | | pyridin-4-yl | | | | | | | | |
| A5 | L1A | bond | piperidin-1-yl | oxo | 2-chloropyridin- | H | H | H | H | H | H | 395 | 2.23 |
| | | | | | 4-yl | | | | | | | | |
| A6 | L1A | bond | piperidin-1-yl | oxo | 2-(piperidin-1- | H | H | H | H | H | H | 472 | 1.63 |
| | | | | | ylcarbonyl) | | | | | | | | |
| | | | | | pyridin-4-yl | | | | | | | | |
| A7 | L1A | bond | pyrrolidin-1-yl | oxo | 2-chloropyridin-4-yl | H | H | H | H | H | H | 381 | 2.08 |
| A8 | L1A | bond | pyrrolidin-1-yl | oxo | 2-(pyrrolidin-1- | H | H | H | H | H | H | 444 | 1.37 |
| | | | | | ylcarbonyl) | | | | | | | | |
| | | | | | pyridin-4-yl | | | | | | | | |
| A9 | L1A | bond | 3-(hydroxy- | oxo | 2-chloropyridin- | H | H | H | H | H | H | 425 | 1.69 |
| | | | methyl) | | 4-yl | | | | | | | | |
| | | | piperidin-1-yl | | | | | | | | | | |
| A10 | L1A | propylimino | 2-hydroxy- | oxo | 2-chloropyridin- | H | H | H | H | H | H | 413 | 1.84 |
| | | | ethyl | | 4-yl | | | | | | | | |
| A11 | L1A | propylimino | propyl | oxo | 2-chloropyridin- | H | H | H | H | H | H | 411 | 2.88^{[c]} |
| | | | | | 4-yl | | | | | | | | |
| A12 | L1A | propylimino | propyl | oxo | 2-(dipropyl- | H | H | H | H | H | H | 504 | 2.54 |
| | | | | | carbamoyl) | | | | | | | | |
| | | | | | pyridin-4-yl | | | | | | | | |
| A13 | L1A | methylimino | propyl | oxo | 2-chloropyridin-4-yl | H | H | H | H | H | H | 383 | 2.2 |
| A14 | L1A | methylimino | propyl | oxo | 2-[methyl(propyl) | H | H | H | H | H | H | 448 | 1.59 |
| | | | | | carbamoyl]-pyridin4-yl | | | | | | | | |
| A15 | L1A | methylimino | tetrahydro- | oxo | 2-chloropyridin- | H | H | H | H | H | H | 425 | 1.99 |
| | | | furan-2-ylmethyl | | 4-yl | | | | | | | | |
| A16 | L1A | methylimino | 2-tetrahy- | oxo | 2-[methyl- | H | H | H | H | H | H | 532 | 1.35 |
| | | | drofuran- | | (tetrahydrofuran-2- | | | | | | | | |
| | | | ylmethyl | | ylmethyl) | | | | | | | | |
| | | | | | carbamoyl] | | | | | | | | |
| | | | | | pyridin-4-yl | | | | | | | | |
| A17 | L1A | methylimino | ethyl | oxo | 2-chloropyridin- | H | H | H | H | H | H | 369 | 1.91 |
| | | | | | 4-yl | | | | | | | | |
| A18 | L1A | methylimino | ethyl | oxo | 2-(trifluoromethyl) | H | H | H | H | H | H | 403 | 2.22 |
| | | | | | pyridin-4-yl | | | | | | | | |
| A19 | L1A | methylimino | ethyl | oxo | 2-[ethyl(methyl) | H | H | H | H | H | H | 420 | 1.19 |
| | | | | | carbamoyl] | | | | | | | | |
| | | | | | pyridin-4-yl | | | | | | | | |
| A20 | L1A | imino | 3-(2-oxoazepan- | oxo | 2-chloropyridin- | H | H | H | H | H | H | 480 | 2.33 |
| | | | 1-yl)propyl | | 4-yl | | | | | | | | |
| A21 | L1A | imino | 3-(2-oxoazepan- | oxo | 2-{[3-(2- | H | H | H | H | H | H | 642 | 2.33 |
| | | | 1-yl)propyl | | oxoazepan- | | | | | | | | |
| | | | | | 1-yl)propyl] | | | | | | | | |
| | | | | | carbamoyl} | | | | | | | | |
| | | | | | pyridin-4-yl | | | | | | | | |
| A22 | L1A | bond | morpholin- | oxo | 2-chloropyridin- | H | H | H | H | H | H | 397 | 1.69 |
| | | | 4-yl | | 4-yl | | | | | | | | |
| A23 | L1A | bond | morpholin- | oxo | 2-(morpholin-4- | H | H | H | H | H | H | 476 | 0.92 |
| | | | 4-yl | | ylcarbonyl) | | | | | | | | |
| | | | | | pyridin-4-yl | | | | | | | | |
| A24 | L1A | methylimino | 2-cyanoethyl | oxo | 2-chloropyridin- | H | H | H | H | H | H | 394 | 1.78 |
| | | | | | 4-yl | | | | | | | | |
| A25 | L1A | methylimino | prop-2-en-1-yl | oxo | 2-chloropyridin- | H | H | H | H | H | H | 381 | 2.13 |
| | | | | | 4-yl | | | | | | | | |
| A26 | L1A | ethylimino | propyl | oxo | 2-chloropyridin- | H | H | H | H | H | H | 397 | 2.51 |
| | | | | | 4-yl | | | | | | | | |
| A27 | L1A | ethylimino | ethyl | oxo | 2-chloropyridin- | H | H | H | H | H | H | 383 | 2.17 |
| | | | | | 4-yl | | | | | | | | |
| A28 | L1A | methylimino | propan-2-yl | oxo | 2-chloropyridin- | H | H | H | H | H | H | 383 | 2.13 |
| | | | | | 4-yl | | | | | | | | |
| A29 | L1A | methylimino | butan-2-yl | oxo | 2-chloropyridin- | H | H | H | H | H | H | 397 | 2.47 |
| | | | | | 4-yl | | | | | | | | |
| A30 | L1A | methylimino | 3-methylbut- | oxo | 2-chloropyridin- | H | H | H | H | H | H | 409 | 2.66 |
| | | | 2-en-1-yl | | 4-yl | | | | | | | | |
| A31 | L1A | methylimino | cyclopropyl- | oxo | 2-chloropyridin- | H | H | H | H | H | H | 395 | 2.28 |
| | | | methyl | | 4-yl | | | | | | | | |
| A32 | L1A | methylimino | methyl | oxo | 2-chloropyridin- | H | H | H | H | H | H | 355 | 1.69 |
| | | | | | 4-yl | | | | | | | | |
| A33 | L1B | ethylimino | ethyl | oxo | 2-chloropyridin- | H | H | H | H | H | H | 383 | 2.6 |
| | | | | | 4-yl | | | | | | | | |
| A34 | L1B | methylimino | methyl | oxo | 2-chloropyridin- | H | H | H | H | H | H | 355 | 1.96^{[b]} |
| | | | | | 4-yl | | | | | | | | |
| A35 | L1B | bond | azetidin-1-yl | oxo | 2-chloropyridin | H | H | H | H | H | H | 367 | 2.21^{[b]} |
| | | | | | -4-yl | | | | | | | | |
| A36 | L1B | methylimino | ethyl | oxo | 2-chloropyridin- | H | H | H | H | H | H | 369 | 2.25^{[b]} |
| | | | | | 4-yl | | | | | | | | |
| A37 | L1A | methylimino | ethyl | oxo | 4-methyl- | H | H | H | H | H | H | 354 | 2.43 |
| | | | | | thiopen-4-yl | | | | | | | | |
| A38 | L1A | ethylimino | ethyl | oxo | thiophen-3-yl | H | H | H | H | H | H | 354 | 2.55 |
| A39 | L1A | ethylimino | ethyl | oxo | 5-tert-butyl-t | H | H | H | H | H | H | 410 | 3.62 |
| | | | | | hiophen-2-yl | | | | | | | | |
| A40 | L1A | ethylimino | ethyl | oxo | 5-(4-fluorophenyl) | H | H | H | H | H | H | 448 | 3.71 |
| | | | | | thiophen-2-yl | | | | | | | | |
| A41 | L1A | ethylimino | ethyl | oxo | thiophen-3-yl | H | H | H | H | H | CH₃ | 368 | 2.61 |
| A45 | L1A | methylimino | 3-methylbut- | oxo | 2-chloro- | H | H | H | R^{a1} | H | H | 481 | 2.49 |
| | | | 2-en-1-yl | | pyridin-4-yl | | | | | | | | |
| A46 | L1A | methylimino | ethyl | oxo | 4,5-dimethyl- | H | H | H | H | H | H | 368 | 2.68 |
| | | | | | thiophen-2-yl | | | | | | | | |
| A47 | L1A | methylimino | ethyl | oxo | 5-methyl- | H | H | H | H | H | H | 354 | 2.39 |
| | | | | | thiophen-2-yl | | | | | | | | |
| A48 | L1A | methylimino | CH₃ | oxo | 5-methyl-t | H | H | H | H | H | H | 340 | 2.13 |
| | | | | | hiophen-2-yl | | | | | | | | |

Abbreviations: R^{a1} = methoxyacetyl;
Measurement of logP values was performed according EEC directive 79/831 Annex V.A8 by HPLC (High Performance Liquid
Chromatography) on reversed phase columns with the following methods:
Usually measurement of LC-MS was done at pH 2.7 with 0.1% formic acid in water and with acetonitrile (contains 0.1% formic acid) as
eluent with a linear gradient from 10% acetonitrle to 95% acetonitrile.
^{[b]}Measurement was done at pH 2.3 with 0.1% phosphoric acid and acetonitrile as eluent.
^{[c]}Measurement with LC-MS was done at pH 7.8 with 0.001 molar ammonium hydrogen carbonate solution in water as eluent with a linear
gradient from 10% acetonitrile to 95% acetonitrile.
Calibration was done with not branched alkan2-ones (with 3 to 16 carbon atoms) with known logP-values (measurement of logP values
using retention times with linear interpolation between successive alkanones) . . . lambda-maX-values were determined using UV-spectra
from 200 nm to 400 nm and the peak values of the chromatographic signals.

TABLE II
Formula (II)

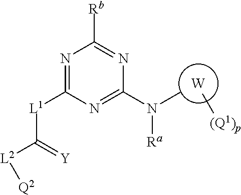

| | In table II we use the same abbreviations for | |
|---|---|---|
| | *—L¹—C(═Y)—L²—Q² as in table I. | |
| | *—L¹—C(═Y)—L²—Q² | Abbreviation |
| 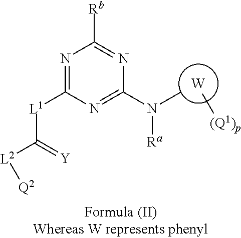 | 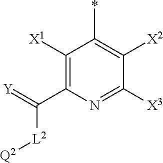 | L1A |

If W(Q¹)ₚ = subst. Phenyl, then the position of (Q¹)ₚ is as shown:

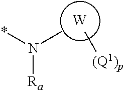

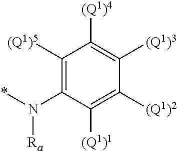

| Ex- | | | | | | | | | | | | | | | | |
|---|---|---|---|---|---|---|---|---|---|---|---|---|---|---|---|---|
| ample | | | | | | | | | | | | | | | Mea- | |
| Num- | | | | | | | | | | | | | | | sured | log |
| ber | L¹C(═Y)L²Q² | L² | Q² | Y | (Q¹⁾¹ | (Q¹⁾² | (Q¹⁾³ | (Q¹⁾⁴ | (Q¹⁾⁵ | R^{a} | R^{b} | X¹ | X² | X³ | MW | p |
| B1 | L1A | methylimino | 2-cyanoethyl | oxo | H | fluoro | H | H | H | H | H | H | H | H | 378 | 2.22 |
| B2 | L1A | ethylimino | ethyl | oxo | H | fluoro | H | H | H | H | H | H | H | H | 367 | 2.72 |
| B3 | L1A | methylimino | propyl | oxo | H | fluoro | H | H | H | H | H | H | H | H | 367 | 2.72 |
| B4 | L1A | methylimino | cyclopropylmethyl | oxo | H | fluoro | H | H | H | H | H | H | H | H | 379 | 2.82 |
| B5 | L1A | methylimino | methyl | oxo | H | fluoro | H | H | H | H | H | H | H | H | 339 | 2.13 |
| B6 | L1A | methylimino | 2-methylpropyl | oxo | H | fluoro | H | H | H | H | H | H | H | H | 381 | 2.92 |
| B7 | L1A | methylimino | ethyl | oxo | H | fluoro | H | H | H | H | H | H | H | H | 353 | 2.4 |
| B8 | L1A | methylimino | propan-2-yl | oxo | H | fluoro | H | H | H | H | H | H | H | H | 367 | 2.67 |
| B9 | L1A | methylimino | 2-methoxyethyl | oxo | H | fluoro | H | H | H | H | H | H | H | H | 383 | 2.26 |
| B10 | L1A | methylimino | ethyl | thioxo | H | fluoro | H | H | H | H | H | H | H | H | 369 | 2.94 |
| B11 | L1A | ethylimino | ethyl | thioxo | H | fluoro | H | H | H | H | H | H | H | H | 383 | 3.31 |
| B12 | L1A | methylimino | cyclopropylmethyl | thioxo | H | fluoro | H | H | H | H | H | H | H | H | 395 | 3.42 |
| B13 | L1A | methylimino | propyl | thioxo | H | fluoro | H | H | H | H | H | H | H | H | 383 | 3.32 |
| B14 | L1A | methylimino | 2-cyanoethyl | oxo | H | H | F | H | H | H | H | H | H | H | 378 | 2.14 |
| B15 | L1A | ethylimino | ethyl | oxo | H | H | F | H | H | H | H | H | H | H | 367 | 2.6 |
| B16 | L1A | methylimino | propyl | oxo | H | H | F | H | H | H | H | H | H | H | 367 | 2.62 |
| B17 | L1A | methylimino | cyclopropylmethyl | oxo | H | H | F | H | H | H | H | H | H | H | 379 | 2.73 |
| B18 | L1A | methylimino | methyl | oxo | H | H | F | H | H | H | H | H | H | H | 339 | 2.02 |
| B19 | L1A | methylimino | 2-methylpropyl | oxo | H | H | F | H | H | H | H | H | H | H | 381 | 2.92 |
| B20 | L1A | methylimino | ethyl | oxo | H | H | F | H | H | H | H | H | H | H | 353 | 2.28 |
| B21 | L1A | methylimino | propan-2-yl | oxo | H | H | F | H | H | H | H | H | H | H | 367 | 2.55 |
| B22 | L1A | methylimino | 2-methoxyethyl | oxo | H | H | F | H | H | H | H | H | H | H | 383 | 3.92 |
| B23 | L1A | methylimino | ethyl | thioxo | H | H | F | H | H | H | H | H | H | H | 369 | 2.8 |
| B24 | L1A | methylimino | 1-cyclopropyl-2-methoxyethyl | oxo | H | H | F | H | H | H | H | H | H | H | 423 | 2.8 |
| B25 | L1A | oxy | methyl | oxo | H | chloro | H | H | H | H | H | H | H | H | 342 | 2.79 |
| B26 | L1A | bond | methyl | oxo | H | chloro | H | H | H | H | H | H | H | H | 326 | 3.32 |
| B27 | L1A | bond | methyl | methoxy-imino | H | chloro | H | H | H | H | H | H | H | H | 355 | 4.23 |
| B28 | L1A | bond | pyrrolidin-1-yl | oxo | H | chloro | H | H | H | H | H | H | H | H | 381 | 2.88 |
| B29 | L1A | bond | methyl | (prop-2-en-1- yloxy) imino | H | chloro | H | H | H | H | H | H | H | H | 381 | 4.7 |
| B30 | L1A | bond | methyl | (but-3-yn-2- yloxy) imino | H | chloro | H | H | H | H | H | H | H | H | 393 | 4.47 |
| B31 | L1A | bond | methyl | ethoxy-imino | H | chloro | H | H | H | H | H | H | H | H | 369 | 4.7 |
| B32 | L1A | bond | methyl | [2- (dimethyl- amino) ethoxy] imino | H | chloro | H | H | H | H | H | H | H | H | 412 | 1.99 |
| B33 | L1A | imino | tetrahydrofuran-2-ylmethyl | oxo | H | chloro | H | H | H | H | H | H | H | H | 411 | 3.09 |
| B34 | L1A | bond | methyl | (cyclo-propyl methoxy) imino | H | chloro | H | H | H | H | H | H | H | H | 395 | 4.94 |
| B35 | L1A | bond | methyl | (hex-2-yn-1- yloxy) imino | H | chloro | H | H | H | H | H | H | H | H | 421 | 5.16 |
| B36 | L1A | bond | methyl | (but-2-yn-1- yloxy) imino | H | chloro | H | H | H | H | H | H | H | H | 393 | 4.41 |
| B37 | L1A | bond | methyl | (prop-2-yn-1- yloxy) imino | H | chloro | H | H | H | H | H | H | H | H | 379 | 3.98 |
| B38 | L1A | bond | methyl | (cyclo-pentyl- oxy) imino | H | chloro | H | H | H | H | H | H | H | H | 409 | 5.79 |
| B39 | L1A | bond | methyl | [(2,6- dichloro-benzyl) oxy]imino | H | chloro | H | H | H | H | H | H | H | H | 499 | 6.02 |
| B40 | L1A | bond | ethyl | ethoxy-imino | H | chloro | H | H | H | H | H | H | H | H | 383 | 5.1 |
| B41 | L1A | bond | ethyl | (prop-2-en-1- yloxy) imino | H | chloro | H | H | H | H | H | H | H | H | 395 | 5.14 |
| B42 | L1A | bond | ethyl | (but-3-yn-2- yloxy) imino | H | chloro | H | H | H | H | H | H | H | H | 407 | 4.87 |
| B43 | L1A | bond | methyl | propoxy-imino | H | chloro | H | H | H | H | H | H | H | H | 383 | 5.13 |
| B44 | L1A | bond | methyl | (2- methyl-propoxy) imino | H | chloro | H | H | H | H | H | H | H | H | 397 | 5.63 |
| B45 | L1A | bond | ethyl | oxo | H | chloro | H | H | H | H | H | H | H | H | 340 | 3.79 |
| B46 | L1A | bond | ethyl | (hex-2-yn-1- yloxy)-imino | H | chloro | H | H | H | H | H | H | H | H | 435 | 5.81 |
| B47 | L1A | bond | ethyl | (cyclo-pentyl- oxy)imino | H | chloro | H | H | H | H | H | H | H | H | 423 | 6.39 |
| B48 | L1A | bond | methyl | (dicyclo-propyl methoxy) imino | H | chloro | H | H | H | H | H | H | H | H | 435 | 5.64 |
| B49 | L1A | bond | ethyl | [2- (dimethyl- amino) ethoxy] imino | H | chloro | H | H | H | H | H | H | H | H | 426 | 2.06 |
| B50 | L1A | bond | methyl | (benzy-loxy) imino | H | chloro | H | H | H | H | H | H | H | H | 431 | 5.18 |
| B51 | L1A | imino | 1-methoxy-propan-2-yl | oxo | H | chloro | H | H | H | H | H | H | H | H | 399 | 3.21 |
| B52 | L1A | imino | (2S)-1-methoxy-propan-2-yl | oxo | H | chloro | H | H | H | H | H | H | H | H | 399 | 3.39 |
| B53 | L1A | bond | ethyl | (but-2-yn-1- yloxy) imino | H | chloro | H | H | H | H | H | H | H | H | 407 | 4.91 |
| B54 | L1A | bond | ethyl | (cyclo-propyl methoxy) imino | H | chloro | H | H | H | H | H | H | H | H | 409 | 5.49 |
| B55 | L1A | bond | ethyl | propoxy-imino | H | chloro | H | H | H | H | H | H | H | H | 397 | 5.69 |
| B56 | L1A | imino | cyclohexyl | oxo | H | chloro | H | H | H | H | H | H | H | H | 409 | 4.46 |
| B57 | L1A | imino | cyclopropyl | oxo | H | chloro | H | H | H | H | H | H | H | H | 367 | 3.05 |
| B58 | L1A | bond | ethyl | (2,2,2- trifluoro-ethoxy) imino | H | chloro | H | H | H | H | H | H | H | H | 437 | 5.39 |
| B59 | L1A | imino | 2-methoxy-ethyl | oxo | H | chloro | H | H | H | H | H | H | H | H | 385 | 2.84 |
| B60 | L1A | bond | methyl | [(4- meth-oxybenzyl) oxy]imino | H | chloro | H | H | H | H | H | H | H | H | 461 | 4.96 |
| B61 | L1A | imino | 3-methylbutyl | oxo | H | chloro | H | H | H | H | H | H | H | H | 397 | 4.3 |
| B62 | L1A | imino | 3-methylbutan-2-yl | oxo | H | chloro | H | H | H | H | H | H | H | H | 397 | 4.3 |
| B63 | L1A | imino | pentan-3-yl | oxo | H | chloro | H | H | H | H | H | H | H | H | 397 | 4.22 |
| B64 | L1A | bond | ethyl | hydroxy-imino | H | chloro | H | H | H | H | H | H | H | H | 355 | 3.24 |
| B65 | L1A | bond | ethyl | (2- methyl-propoxy) imino | H | chloro | H | H | H | H | H | H | H | H | 411 | 6.19 |
| B66 | L1A | imino | 1-(dimethylamino)propan-2-yl | oxo | H | chloro | H | H | H | H | H | H | H | H | 412 | 1.72 |
| B67 | L1A | imino | pentyl | oxo | H | chloro | H | H | H | H | H | H | H | H | 397 | 4.35 |
| B68 | L1A | bond | ethyl | [(4- methoxy-benzyl) oxy]imino | H | chloro | H | H | H | H | H | H | H | H | 475 | 5.46 |
| B69 | L1A | bond | ethyl | [(2,6- dichloro-benzyl) oxy]imino | H | chloro | H | H | H | H | H | H | H | H | 513 | 6.53 |
| B70 | L1A | bond | ethyl | [(4- chloro-benzyl) oxy]imino | H | chloro | H | H | H | H | H | H | H | H | 479 | 6.22 |
| B71 | L1A | imino | cyclopentyl | oxo | H | chloro | H | H | H | H | H | H | H | H | 395 | 4.03 |
| B72 | L1A | bond | ethyl | (benzyl-oxy)imino | H | chloro | H | H | H | H | H | H | H | H | 445 | 5.65 |
| B73 | L1A | bond | methyl | (2,2,2- trifluoro-ethoxy) imino | H | chloro | H | H | H | H | H | H | H | H | 423 | 4.58 |
| B74 | L1A | bond | methyl | hydroxy-imino | H | chloro | H | H | H | H | H | H | H | H | 341 | 2.8 |
| B75 | L1A | bond | methyl | 2- methyl-hydra- zinylidene | H | chloro | H | H | H | H | H | H | H | H | 354 | 2.27 |
| B76 | L1A | bond | methyl | 2-(2,2,2- trifluoro-ethyl) hydrazin-ylidene | H | chloro | H | H | H | H | H | H | H | H | 422 | 3.58 |
| B77 | L1A | bond | methyl | 2-(2-ethoxy-2- oxoethyl) hydrazin-ylidene | H | chloro | H | H | H | H | H | H | H | H | 426 | 3.02 |
| B78 | L1A | bond | amino | hydroxy-imino | H | chloro | H | H | H | H | H | H | H | H | 342 | 2.28 |
| B79 | L1A | bond | ethyl | (dicyclo-propyl methoxy) imino | H | chloro | H | H | H | H | H | H | H | H | 449 | 6.59 |
| B80 | L1A | bond | ethyl | (prop-2-yn-1- yloxy) imino | H | chloro | H | H | H | H | H | H | H | H | 393 | 4.73 |
| B81 | L1A | bond | methyl | 2-(2- cyano-ethyl) hydrazin-ylidene | H | chloro | H | H | H | H | H | H | H | H | 393 | 2.5 |
| B82 | L1A | bond | methyl | 2-(4- chloro-phenyl) hydrazin-ylidene | H | chloro | H | H | H | H | H | H | H | H | 450 | 4.97 |
| B83 | L1A | bond | methyl | 2- (methoxy- carbon-yl) hydrazin-ylidene | H | chloro | H | H | H | H | H | H | H | H | 398 | 2.86 |
| B84 | L1A | bond | ethyl | (1- cyclo-propyl ethoxy) imino | H | chloro | H | H | H | H | H | H | H | H | 423 | 6.24 |
| B85 | L1A | bond | ethyl | methoxy-imino | H | chloro | H | H | H | H | H | H | H | H | 369 | 4.81 |
| B86 | L1A | imino | 1,3-dimethoxypropan-2-yl | oxo | H | chloro | H | H | H | H | H | H | H | H | 429 | 3.39 |
| B87 | L1A | imino | 3-(2-oxoazepan-2-yl | oxo | H | chloro | H | H | H | H | H | H | H | H | 480 | 3.11 |
| B88 | L1A | imino | 3-methoxypropyl | oxo | H | chloro | H | H | H | H | H | H | H | H | 399 | 3.15 |
| B89 | L1A | imino | 1-methoxybutan-2-yl | oxo | H | chloro | H | H | H | H | H | H | H | H | 413 | 3.8 |
| B90 | L1A | bond | methyl | chloro-5- (trifluoro- methyl) pyridin-2- yl] hydrazin-ylidene | H | chloro | H | H | H | H | H | H | H | H | 519 | 5.46 |
| B91 | L1A | imino | propan-2-yl | oxo | H | chloro | H | H | H | H | H | H | H | H | 369 | 3.62 |
| B92 | L1A | imino | ethyl | oxo | H | chloro | H | H | H | H | H | H | H | H | 355 | 3.17 |
| B93 | L1A | imino | butyl | oxo | H | chloro | H | H | H | H | H | H | H | H | 383 | 4.11 |
| B94 | L1A | imino | butan-2-yl | oxo | H | chloro | H | H | H | H | H | H | H | H | 383 | 4.04 |
| B95 | L1A | imino | 1-cyanobutan-2-yl | oxo | H | chloro | H | H | H | H | H | H | H | H | 408 | 3.35 |
| B96 | L1A | imino | 4-(2-oxopyrrolidin-1-yl)butyl | oxo | H | chloro | H | H | H | H | H | H | H | H | 466 | 2.73 |
| B97 | L1A | imino | 4-ethoxycyclohexyl | oxo | H | chloro | H | H | H | H | H | H | H | H | 453 | 4.39 |
| B98 | L1A | imino | tert-butyl | oxo | H | chloro | H | H | H | H | H | H | H | H | 383 | 4.39 |
| B99 | L1A | imino | cyclopropylmethyl | oxo | H | chloro | H | H | H | H | H | H | H | H | 381 | 3.73 |
| B100 | L1A | imino | 3-(2-oxopyrrolidin-1-yl)propyl | oxo | H | chloro | H | H | H | H | H | H | H | H | 452 | 2.63 |
| B101 | L1A | imino | 1,1-dioxidotetrahydrothiophen-3-yl | oxo | H | chloro | H | H | H | H | H | H | H | H | 445 | 2.66 |
| B102 | L1A | bond | ethoxy | imino | H | chloro | H | H | H | H | H | H | H | H | 355 | 2.08 |
| B103 | L1A | imino | propyl | oxo | H | chloro | H | H | H | H | H | H | H | H | 369 | 3.62 |
| B104 | L1A | bond | amino | [(methoxy- acetyl) oxy]imino | H | chloro | H | H | H | H | H | H | H | H | 414 | 2.92 |
| B105 | L1A | imino | 2-(morpholin-4-yl)ethyl | oxo | H | chloro | H | H | H | H | H | H | H | H | 440 | 1.68 |
| B106 | L1A | bond | amino | (butan-oyloxy) imino | H | chloro | H | H | H | H | H | H | H | H | 412 | 3.8 |
| B107 | L1A | bond | amino | [(2- methyl- propanoyl) oxy]imino | H | chloro | H | H | H | H | H | H | H | H | 412 | 3.69 |
| B108 | L1A | bond | amino | (pro-panoyl) imino | H | chloro | H | H | H | H | H | H | H | H | 398 | 3.33 |
| B109 | L1A | bond | amino | (acetyloxy) imino | H | chloro | H | H | H | H | H | H | H | H | 384 | 2.95 |
| B110 | L1A | bond | amino | [(3- methyl- butanoyl) oxy]imino | H | chloro | H | H | H | H | H | H | H | H | 426 | 4.12 |
| B111 | L1A | bond | amino | [(3-methylbut- 2-enoyl) oxy]imino | H | chloro | H | H | H | H | H | H | H | H | 424 | 3.92 |
| B112 | L1A | imino | oxetan-3-yl | oxo | H | chloro | H | H | H | H | H | H | H | H | 383 | 2.6 |
| B113 | L1A | imino | cyclobutyl | oxo | H | chloro | H | H | H | H | H | H | H | H | 381 | 3.89 |
| B114 | L1A | imino | 1-(diethylamino)propan-2-yl | oxo | H | chloro | H | H | H | H | H | H | H | H | 440 | 1.9 |
| B115 | L1A | bond | piperidin-1-yl | oxo | H | chloro | H | H | H | H | H | H | H | H | 395 | 3.1 |
| B116 | L1A | bond | morpholin-4-yl | oxo | H | chloro | H | H | H | H | H | H | H | H | 397 | 2.39 |
| B117 | L1A | imino | 2,2,2-trifluoroethyl | oxo | H | chloro | H | H | H | H | H | H | H | H | 409 | 3.67 |
| B118 | L1A | imino | pentan-2-yl | oxo | H | chloro | H | H | H | H | H | H | H | H | 397 | 4.54 |
| B119 | L1A | imino | H | thioxo | H | chloro | H | H | H | H | H | H | H | H | 343 | 3.12 |
| B120 | L1A | bond | 4-(trifluoro-methyl)piperidin-1-yl | oxo | H | chloro | H | H | H | H | H | H | H | H | 463 | 3.55 |
| B121 | L1A | imino | 3,3,3-trifluoropropyl | oxo | H | chloro | H | H | H | H | H | H | H | H | 423 | 3.73 |
| B122 | L1A | imino | 1-ethyl-piperidin-3-yl | oxo | H | chloro | H | H | H | H | H | H | H | H | 438 | 1.82 |
| B123 | L1A | methylimino | butyl | oxo | H | chloro | H | H | H | H | H | H | H | H | 396 | 3.48 |
| B124 | L1A | propylimino | propyl | oxo | H | chloro | H | H | H | H | H | H | H | H | 410 | 3.89 |
| B125 | L1A | ethylimino | propyl | oxo | H | chloro | H | H | H | H | H | H | H | H | 396 | 3.46 |
| B126 | L1A | propylimino | butyl | oxo | H | chloro | H | H | H | H | H | H | H | H | 425 | 4.31 |
| B127 | L1A | methylimino | prop-2-en-1-yl | oxo | H | chloro | H | H | H | H | H | H | H | H | 381 | 2.96 |
| B128 | L1A | methylimino | hexyl | oxo | H | chloro | H | H | H | H | H | H | H | H | 425 | 4.34 |
| B129 | L1A | methylimino | 2-cyanoethyl | oxo | H | chloro | H | H | H | H | H | H | H | H | 394 | 2.51 |
| B130 | L1A | ethylimino | ethyl | oxo | H | chloro | H | H | H | H | H | H | H | H | 383 | 3.06 |
| B131 | L1A | bond | 4-methyl-piperidin-1-yl | oxo | H | chloro | H | H | H | H | H | H | H | H | 409 | 3.51 |
| B132 | L1A | methylimino | propyl | oxo | H | chloro | H | H | H | H | H | H | H | H | 383 | 3.06 |
| B133 | L1A | methylimino | cyclopropyl-methyl | oxo | H | chloro | H | H | H | H | H | H | H | H | 395 | 3.15 |
| B134 | L1A | bond | (2R)-2-(methoxy-methyl)pyrrolidin-1-yl | oxo | H | chloro | H | H | H | H | H | H | H | H | 425 | 3.08 |
| B135 | L1A | bond | 3-methylpiperidin-1-yl | oxo | H | chloro | H | H | H | H | H | H | H | H | 409 | 3.48 |
| B136 | L1A | methylimino | 2-hydroxyethyl | oxo | H | chloro | H | H | H | H | H | H | H | H | 385 | 2.04 |
| B137 | L1A | bond | 2,3-dimethylpiperidin-1-yl | oxo | H | chloro | H | H | H | H | H | H | H | H | 423 | 3.78 |
| B138 | L1A | bond | azepan-1-yl | oxo | H | chloro | H | H | H | H | H | H | H | H | 409 | 3.46 |
| B139 | L1A | bond | 2-ethylpiperidin-1-yl | oxo | H | chloro | H | H | H | H | H | H | H | H | 423 | 3.8 |
| B140 | L1A | bond | 3-hydroxypiperidin-1-yl | oxo | H | chloro | H | H | H | H | H | H | H | H | 411 | 2.2 |
| B141 | L1A | bond | 2,5-dimethylpyrrolidin-1-yl | oxo | H | chloro | H | H | H | H | H | H | H | H | 409 | 3.64 |
| B142 | L1A | bond | 2-methyl-piperidin-1-yl | oxo | H | chloro | H | H | H | H | H | H | H | H | 409 | 3.39 |
| B143 | L1A | prop-2-en-1-ylimino | prop-2-en-1-yl | oxo | H | chloro | H | H | H | H | H | H | H | H | 407 | 3.67 |
| B144 | L1A | methylimino | cyclohexyl | oxo | H | chloro | H | H | H | H | H | H | H | H | 423 | 3.89 |
| B145 | L1A | bond | 3-(hydroxymethyl)piperidin-1-yl | oxo | H | chloro | H | H | H | H | H | H | H | H | 425 | 2.3 |
| B146 | L1A | methylimino | 3-methylbutyl | oxo | H | chloro | H | H | H | H | H | H | H | H | 411 | 3.87 |
| B147 | L1A | bond | 3,3-dimethylpiperidin-1-yl | oxo | H | chloro | H | H | H | H | H | H | H | H | 423 | 3.83 |
| B148 | L1A | methylimino | 3-methylbut-2-en-1-yl | oxo | H | chloro | H | H | H | H | H | H | H | H | 409 | 3.67 |
| B149 | L1A | methylimino | cyclopentyl | oxo | H | chloro | H | H | H | H | H | H | H | H | 409 | 3.51 |
| B150 | L1A | methylimino | methyl | oxo | H | chloro | H | H | H | H | H | H | H | H | 355 | 2.4 |
| B151 | L1A | methylimino | 2-methyl-propyl | oxo | H | chloro | H | H | H | H | H | H | H | H | 397 | 3.42 |
| B152 | L1A | bond | 2-methylpyrrolidin-1-yl | oxo | H | chloro | H | H | H | H | H | H | H | H | 395 | 3.23 |
| B153 | L1A | bond | 3,6-dihydropyridin-1(2H)-yl | oxo | H | chloro | H | H | H | H | H | H | H | H | 393 | 3.11 |
| B154 | L1A | bond | 2,6-dimethylmorpholin-4-yl | oxo | H | chloro | H | H | H | H | H | H | H | H | 425 | 2.92 |
| B155 | L1A | bond | 3,5-dimethyl-piperidin-1-yl | oxo | H | chloro | H | H | H | H | H | H | H | H | 423 | 3.96 |
| B156 | L1A | ethylimino | 2-methylprop-2-en-1-yl | oxo | H | chloro | H | H | H | H | H | H | H | H | 409 | 3.67 |
| B157 | L1A | methylimino | ethyl | oxo | H | chloro | H | H | H | H | H | H | H | H | 369 | 2.69 |
| B158 | L1A | methylimino | propan-2-yl | oxo | H | chloro | H | H | H | H | H | H | H | H | 383 | 2.96 |
| B159 | L1A | propylimino | cyclopropyl-methyl | oxo | H | chloro | H | H | H | H | H | H | H | H | 423 | 3.94 |
| B160 | L1A | methylimino | tetrahydro-furan-2-ylmethyl | oxo | H | chloro | H | H | H | H | H | H | H | H | 425 | 2.76 |
| B161 | L1A | methylimino | pentyl | oxo | H | chloro | H | H | H | H | H | H | H | H | 411 | 3.89 |
| B162 | L1A | ethylimino | butyl | oxo | H | chloro | H | H | H | H | H | H | H | H | 411 | 3.87 |
| B163 | L1A | bond | 4-formyl-piperazin-1-yl | oxo | H | chloro | H | H | H | H | H | H | H | H | 424 | 2.04 |
| B164 | L1A | ethylimino | 2-hydroxyethyl | oxo | H | chloro | H | H | H | H | H | H | H | H | 399 | 2.25 |
| B165 | L1A | bond | 2-(hydroxymethyl)piperidin-1-yl | oxo | H | chloro | H | H | H | H | H | H | H | H | 425 | 2.48 |
| B166 | L1A | ethylimino | 2-hydroxy-2-methylpropyl | oxo | H | chloro | H | H | H | H | H | H | H | H | 427 | 2.66 |
| B167 | L1A | ethylimino | tetrahydro-furan-2-yl-methyl | oxo | H | chloro | H | H | H | H | H | H | H | H | 439 | 3.09 |
| B168 | L1A | bond | 4-oxoimidazo-lidin-1-yl | oxo | H | chloro | H | H | H | H | H | H | H | H | 396 | 2.15 |
| B169 | L1A | bond | 4-methoxypiperidin-1-yl | oxo | H | chloro | H | H | H | H | H | H | H | H | 425 | 2.68 |
| B170 | L1A | bond | 4-cyano-piperidin-1-yl | oxo | H | chloro | H | H | H | H | H | H | H | H | 420 | 2.59 |
| B171 | L1A | propylimino | butan-2-yl | oxo | H | chloro | H | H | H | H | H | H | H | H | 425 | 4.13 |
| B172 | L1A | methylimino | 2-methoxy-ethyl | oxo | H | chloro | H | H | H | H | H | H | H | H | 399 | 2.57 |
| B173 | L1A | bond | 4-hydroxy-piperidin-1-yl | oxo | H | chloro | H | H | H | H | H | H | H | H | 411 | 2.04 |
| B174 | L1A | hexylimino | hexyl | oxo | H | chloro | H | H | H | H | H | H | H | H | 496 | 6.69 |
| B175 | L1A | butylimino | butyl | oxo | H | chloro | H | H | H | H | H | H | H | H | 439 | 4.75 |
| B176 | L1A | bond | thiomorpholin-4-yl | oxo | H | chloro | H | H | H | H | H | H | H | H | 413 | 2.98 |
| B177 | L1A | bond | 3-hydroxypyrrolidin-1-yl | oxo | H | chloro | H | H | H | H | H | H | H | H | 397 | 2.07 |
| B178 | L1A | imino | 3-(formylamino)propyl | oxo | H | chloro | H | H | H | H | H | H | H | H | 394 | 2.26 |
| B179 | L1A | bond | 3-methoxypiperidin-1-yl | oxo | H | chloro | H | H | H | H | H | H | H | H | 424 | 2.76[b] |
| B180 | L1A | (2-hydroxyethyl)imino | propan-2-yl | oxo | H | chloro | H | H | H | H | H | H | H | H | 413 | 2.45 |
| B181 | L1A | propylimino | 2-hydroxyethyl | oxo | H | chloro | H | H | H | H | H | H | H | H | 413 | 2.51[b] |
| B182 | L1A | propylimino | 2-cyanoethyl | oxo | H | chloro | H | H | H | H | H | H | H | H | 422 | 3.13[b] |
| B183 | L1A | imino | 3-hydroxypropyl | oxo | H | chloro | H | H | H | H | H | H | H | H | 385 | 2.4 |
| B184 | L1A | bond | 4-methylpiperazin-1-yl | oxo | H | chloro | H | H | H | H | H | H | H | H | 410 | 1.5 |
| B185 | L1A | imino | hydroxy | oxo | H | chloro | H | H | H | H | H | H | H | H | 343 | 2.13 |
| B186 | L1A | methylimino | methoxy | oxo | H | chloro | H | H | H | H | H | H | H | H | 371 | 2.59 |

| B187 | L1A | 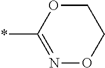 | | | H | chloro | H | H | H | H | H | H | H | H | 369 | 2.63 |
|---|---|---|---|---|---|---|---|---|---|---|---|---|---|---|---|---|
| B188 | L1A | methylimino | 1-cyclopropyl-2-methoxyethyl | oxo | H | chloro | H | H | H | H | H | H | H | H | 480 | 3.09 |
| B189 | L1A | methylimino | 3-methoxybutan-2-yl | oxo | H | chloro | H | H | H | H | H | H | H | H | 427 | 3.15 |
| B190 | L1A | bond | pyrrolidin-1-yl | oxo | H | chloro | F | H | H | H | H | H | H | H | 399 | 3 |
| B191 | L1A | imino | 1-methoxy-propan-2-yl | oxo | H | chloro | F | H | H | H | H | H | H | H | 417 | 3.48 |
| B192 | L1A | imino | cyclopropyl | oxo | H | chloro | F | H | H | H | H | H | H | H | 385 | 3.31 |
| B193 | L1A | imino | 2-methoxyethyl | oxo | H | chloro | F | H | H | H | H | H | H | H | 403 | 3.08 |
| B194 | L1A | imino | 3-(2-oxoazepan-1-yl)propyl | oxo | H | chloro | F | H | H | H | H | H | H | H | 498 | 3.19 |
| B195 | L1A | imino | 1-methoxybutan-2-yl | oxo | H | chloro | F | H | H | H | H | H | H | H | 431 | 3.89 |
| B196 | L1A | imino | propan-2-yl | oxo | H | chloro | F | H | H | H | H | H | H | H | 387 | 3.73 |
| B197 | L1A | imino | butan-2-yl | oxo | H | chloro | F | H | H | H | H | H | H | H | 401 | 4.16 |
| B198 | L1A | imino | cyclopropylmethyl | oxo | H | chloro | F | H | H | H | H | H | H | H | 399 | 3.83 |
| B199 | L1A | imino | cyclobutyl | oxo | H | chloro | F | H | H | H | H | H | H | H | 399 | 3.94 |
| B200 | L1A | imino | pentan-2-yl | oxo | H | chloro | F | H | H | H | H | H | H | H | 415 | 4.64 |
| B201 | L1A | methylimino | 2-cyanoethyl | oxo | H | chloro | F | H | H | H | H | H | H | H | 412 | 2.58 |
| B202 | L1A | ethylimino | ethyl | oxo | H | chloro | F | H | H | H | H | H | H | H | 401 | 3.13 |
| B203 | L1A | methylimino | propyl | oxo | H | chloro | F | H | H | H | H | H | H | H | 401 | 3.13 |
| B204 | L1A | methylimino | cyclopropylmethyl | oxo | H | chloro | F | H | H | H | H | H | H | H | 413 | 3.23 |
| B205 | L1A | methylimino | methyl | oxo | H | chloro | F | H | H | H | H | H | H | H | 373 | 2.47 |
| B206 | L1A | methylimino | 2-methylpropyl | oxo | H | chloro | F | H | H | H | H | H | H | H | 415 | 3.46 |
| B207 | L1A | methylimino | ethyl | oxo | H | chloro | F | H | H | H | H | H | H | H | 387 | 2.75 |
| B208 | L1A | methylimino | propan-2-yl | oxo | H | chloro | F | H | H | H | H | H | H | H | 401 | 3.02 |
| B209 | L1A | methylimino | ethyl | thioxo | H | chloro | F | H | H | H | H | H | H | H | 403 | 3.33 |
| B210 | L1A | methylimino | 1-cyclopropyl-2-methoxyethyl | oxo | H | chloro | F | H | H | H | H | H | H | H | 457 | 3.33 |
| B211 | L1A | bond | pyrrolidin-1-yl | oxo | H | methyl-sulfanyl | H | H | H | H | H | H | H | H | 393 | 2.82 |
| B212 | L1A | imino | 1-methoxypropan-2-yl | oxo | H | methyl-sulfanyl | H | H | H | H | H | H | H | H | 411 | 3.31 |
| B213 | L1A | imino | cyclopropyl | oxo | H | methyl-sulfanyl | H | H | H | H | H | H | H | H | 379 | 3.15 |
| B214 | L1A | imino | 2-methoxyethyl | oxo | H | methyl-sulfanyl | H | H | H | H | H | H | H | H | 397 | 2.92 |
| B215 | L1A | imino | 3-(2-oxoazepan-1-yl)propyl | oxo | H | methyl-sulfanyl | H | H | H | H | H | H | H | H | 492 | 3.02 |
| B216 | L1A | imino | 1-methoxybutan-2-yl | oxo | H | methyl-sulfanyl | H | H | H | H | H | H | H | H | 425 | 3.69 |
| B217 | L1A | imino | propan-2-yl | oxo | H | methyl-sulfanyl | H | H | H | H | H | H | H | H | 381 | 3.53 |
| B218 | L1A | imino | butan-2-yl | oxo | H | methyl-sulfanyl | H | H | H | H | H | H | H | H | 395 | 3.94 |
| B219 | L1A | imino | cyclopropylmethyl | oxo | H | methyl-sulfanyl | H | H | H | H | H | H | H | H | 393 | 3.64 |
| B220 | L1A | imino | cyclobutyl | oxo | H | methyl sulfanyl | H | H | H | H | H | H | H | H | 393 | 3.73 |
| B221 | L1A | imino | pentan-2-yl | oxo | H | methyl sulfanyl | H | H | H | H | H | H | H | H | 409 | 4.44 |
| B222 | L1A | methylimino | ethyl | oxo | H | methyl sulfanyl | H | H | H | H | H | H | H | H | 381 | 2.6 |
| B223 | L1A | methylimino | ethyl | thioxo | H | methyl sulfanyl | H | H | H | H | H | H | H | H | 397 | 3.17 |
| B224 | L1A | bond | pyrrolidin-1-yl | oxo | H | chloro | Me | H | H | H | H | H | H | H | 395 | 3.26 |
| B225 | L1A | imino | 1-methoxypropan-2-yl | oxo | H | chloro | Me | H | H | H | H | H | H | H | 413 | 3.8 |
| B226 | L1A | imino | cyclopropyl | oxo | H | chloro | Me | H | H | H | H | H | H | H | 381 | 3.62 |
| B227 | L1A | imino | 2-methoxyethyl | oxo | H | chloro | Me | H | H | H | H | H | H | H | 399 | 3.48 |
| B228 | L1A | imino | 3-(2-oxoazepan-1-yl)propyl | oxo | H | chloro | Me | H | H | H | H | H | H | H | 494 | 3.44 |
| B229 | L1A | imino | 1-methoxybutan-2-yl | oxo | H | chloro | Me | H | H | H | H | H | H | H | 427 | 4.24 |
| B230 | L1A | imino | propan-2-yl | oxo | H | chloro | Me | H | H | H | H | H | H | H | 383 | 4.04 |
| B231 | L1A | imino | ethyl | oxo | H | chloro | Me | H | H | H | H | H | H | H | 369 | 3.55 |
| B232 | L1A | imino | butan-2-yl | oxo | H | chloro | Me | H | H | H | H | H | H | H | 397 | 4.49 |
| B233 | L1A | imino | cyclopropylmethyl | oxo | H | chloro | Me | H | H | H | H | H | H | H | 395 | 4.16 |
| B234 | L1A | imino | cyclobutyl | oxo | H | chloro | Me | H | H | H | H | H | H | H | 395 | 4.29 |
| B235 | L1A | imino | pentan-2-yl | oxo | H | chloro | Me | H | H | H | H | H | H | H | 411 | 5 |
| B236 | L1A | methylimino | ethyl | oxo | H | chloro | Me | H | H | H | H | H | H | H | 383 | 3.02 |
| B237 | L1A | methylimino | 2-cyanoethyl | oxo | fluoro | H | H | fluoro | H | H | H | H | H | H | 396 | 2.1 |
| B238 | L1A | methylimino | 2-methylpropyl | oxo | fluoro | H | H | fluoro | H | H | H | H | H | H | 399 | 2.9 |
| B239 | L1A | methylimino | 2-cyanoethyl | oxo | H | methoxy-methyl | H | H | H | H | H | H | H | H | 404 | 1.94 |
| B240 | L1A | ethylimino | ethyl | oxo | H | methoxy-methyl | H | H | H | H | H | H | H | H | 393 | 2.48 |
| B241 | L1A | methylimino | propyl | oxo | H | methoxy-methyl | H | H | H | H | H | H | H | H | 393 | 2.48 |
| B242 | L1A | methylimino | cyclopropylmethyl | oxo | H | methoxy-methyl | H | H | H | H | H | H | H | H | 405 | 2.58 |
| B243 | L1A | methylimino | methyl | oxo | H | methoxy-methyl | H | H | H | H | H | H | H | H | 365 | 1.91 |
| B244 | L1A | methylimino | 2-methylpropyl | oxo | H | methoxy-methyl | H | H | H | H | H | H | H | H | 407 | 2.66 |
| B245 | L1A | methylimino | ethyl | oxo | H | methoxy-methyl | H | H | H | H | H | H | H | H | 379 | 2.17 |
| B246 | L1A | methylimino | propan-2-yl | oxo | H | methoxy-methyl | H | H | H | H | H | H | H | H | 393 | 2.42 |
| B247 | L1A | methylimino | 2-methoxyethyl | oxo | H | methoxy-methyl | H | H | H | H | H | H | H | H | 409 | 2.05 |
| B248 | L1A | methylimino | ethyl | thioxo | H | methoxy-methyl | H | H | H | H | H | H | H | H | 395 | 2.66 |
| B249 | L1A | methylimino | cyclopropylmethyl | thioxo | H | methoxy-methyl | H | H | H | H | H | H | H | H | 421 | 3.12 |
| B250 | L1A | methylimino | propyl | thioxo | H | methoxy-methyl | H | H | H | H | H | H | H | H | 409 | 3.05 |
| B251 | L1A | methylimino | 1-cyclopropyl-2-methoxyethyl | oxo | H | methoxy-methyl | H | H | H | H | H | H | H | H | 449 | 2.69 |
| B252 | L1A | methylimino | ethyl | oxo | H | pentafluoro- lambda⁶-sulfanyl | H | H | H | H | H | H | H | H | 461 | 3.06 |
| B253 | L1A | methylimino | ethyl | thioxo | H | pentafluoro- lambda⁶- sulfanyl | H | H | H | H | H | H | H | H | 477 | 3.57 |
| B254 | L1A | methylimino | ethyl | oxo | H | ethoxy | H | H | H | H | H | H | H | H | 379 | 2.6 |
| B255 | L1A | methylimino | ethyl | thioxo | H | ethoxy | H | H | H | H | H | H | H | H | 395 | 3.15 |
| B256 | L1A | methylimino | ethyl | oxo | H | trifluoro-methyl | H | H | H | H | H | H | H | H | 403 | 2.86 |
| B257 | L1A | methylimino | ethyl | thioxo | H | trifluoro-methyl | H | H | H | H | H | H | H | H | 419 | 3.41 |
| B258 | L1A | methylimino | ethyl | oxo | H | propan-2-yl | H | H | H | H | H | H | H | H | 377 | 3.11 |
| B259 | L1A | methylimino | ethyl | thioxo | H | propan-2-yl | H | H | H | H | H | H | H | H | 393 | 3.7 |
| B260 | L1A | methylimino | ethyl | oxo | H | 1,1,2,2- tetrafluoro ethoxy | H | H | H | H | H | H | H | H | 451 | 2.92 |
| B261 | L1A | methylimino | ethyl | thioxo | H | 1,1,2,2- tetrfluoro ethoxy | H | H | H | H | H | H | H | H | 467 | 3.41 |
| B262 | L1A | methylimino | 2-cyanoethyl | oxo | H | trifluoro- methoxy | H | H | H | H | H | H | H | H | 444 | 2.8 |
| B263 | L1A | ethylimino | ethyl | oxo | H | trifluoro- methoxy | H | H | H | H | H | H | H | H | 433 | 3.39 |
| B264 | L1A | methylimino | propyl | oxo | H | trifluoro- methoxy | H | H | H | H | H | H | H | H | 433 | 3.37 |
| B265 | L1A | methylimino | cyclopropylmethyl | oxo | H | trifluoro- methoxy | H | H | H | H | H | H | H | H | 445 | 3.48 |
| B266 | L1A | methylimino | methyl | oxo | H | trifluoro- methoxy | H | H | H | H | H | H | H | H | 405 | 2.7 |
| B267 | L1A | methylimino | 2-methylpropyl | oxo | H | trifluoro- methoxy | H | H | H | H | H | H | H | H | 447 | 3.73 |
| B268 | L1A | methylimino | ethyl | oxo | H | trifluoro- methoxy | H | H | H | H | H | H | H | H | 419 | 3 |
| B269 | L1A | methylimino | propan-2-yl | oxo | H | trifluoro- methoxy | H | H | H | H | H | H | H | H | 433 | 3.29 |
| B270 | L1A | methylimino | 2-methoxyethyl | oxo | H | trifluoro- methoxy | H | H | H | H | H | H | H | H | 449 | 2.86 |
| B271 | L1A | methylimino | ethyl | thioxo | H | trifluoro- methoxy | H | H | H | H | H | H | H | H | 435 | 3.57 |
| B272 | L1A | methylimino | 2-cyanoethyl | oxo | H | methoxy | H | H | H | H | H | H | H | H | 390 | 2.11 |
| B273 | L1A | ethylimino | ethyl | oxo | H | methoxy | H | H | H | H | H | H | H | H | 379 | 2.59 |
| B274 | L1A | methylimino | propyl | oxo | H | methoxy | H | H | H | H | H | H | H | H | 379 | 2.61 |
| B275 | L1A | methylimino | cyclopropylmethyl | oxo | F | methoxy | H | H | H | H | H | H | H | H | 391 | 2.7 |
| B276 | L1A | methylimino | methyl | oxo | H | methoxy | H | H | H | H | H | H | H | H | 351 | 2.01 |
| B277 | L1A | methylimino | 2-methylpropyl | oxo | H | methoxy | H | H | H | H | H | H | H | H | 393 | 2.90 |
| B278 | L1A | methylimino | ethyl | oxo | H | methoxy | H | H | H | H | H | H | H | H | 365 | 2.26 |
| B279 | L1A | methylimino | propan-2-yl | oxo | H | methoxy | H | H | H | H | H | H | H | H | 379 | 2.51 |
| B280 | L1A | methylimino | 2-methoxyethyl | oxo | H | methoxy | H | H | H | H | H | H | H | H | 395 | 2.14 |
| B281 | L1A | methylimino | ethyl | thioxo | H | methoxy | H | H | H | H | H | H | H | H | 381 | 2.78 |
| B282 | L1A | ethylimino | ethyl | thioxo | H | methoxy | H | H | H | H | H | H | H | H | 395 | 3.15 |
| B283 | L1A | methylimino | propyl | thioxo | H | methoxy | H | H | H | H | H | H | H | H | 395 | 3.15 |
| B284 | L1A | methylimino | 2-cyanoethyl | oxo | H | difluoro-methyl | H | H | H | H | H | H | H | H | 410 | 2.25 |
| B285 | L1A | ethylimino | ethyl | oxo | H | difluoro-methyl | H | H | H | H | H | H | H | H | 399 | 2.73 |
| B286 | L1A | methylimino | propyl | oxo | H | difluoro-methyl | H | H | H | H | H | H | H | H | 399 | 2.73 |
| B287 | L1A | methylimino | cyclopropylmethyl | oxo | H | difluoro-methyl | H | H | H | H | H | H | H | H | 411 | 2.82 |
| B288 | L1A | methylimino | methyl | oxo | H | difluoro-methyl | H | H | H | H | H | H | H | H | 371 | 2.17 |
| B289 | L1A | methylimino | 2-methylpropyl | oxo | H | difluoro-methyl | H | H | H | H | H | H | H | H | 413 | 3.04 |
| B290 | L1A | methylimino | propan-2-yl | oxo | H | difluoro-methyl | H | H | H | H | H | H | H | H | 399 | 2.65 |
| B291 | L1A | methylimino | 2-cyanoethyl | oxo | H | cyano | H | H | H | H | H | H | H | H | 385 | 1.99 |
| B292 | L1A | ethylimino | ethyl | oxo | H | cyano | H | H | H | H | H | H | H | H | 374 | 2.45 |
| B293 | L1A | methylimino | propyl | oxo | H | cyano | H | H | H | H | H | H | H | H | 374 | 2.45 |
| B294 | L1A | methylimino | methyl | oxo | H | cyano | H | H | H | H | H | H | H | H | 346 | 1.88 |
| B295 | L1A | methylimino | 2-methylpropyl | oxo | H | cyano | H | H | H | H | H | H | H | H | 388 | 2.73 |
| B296 | L1A | methylimino | propan-2-yl | oxo | H | cyano | H | H | H | H | H | H | H | H | 374 | 2.37 |
| B297 | L1A | methylimino | 2-cyanoethyl | oxo | H | difluoro- methoxy | H | H | H | H | H | H | H | H | 426 | 2.37 |
| B298 | L1A | ethylimino | ethyl | oxo | H | difluoro- methoxy | H | H | H | H | H | H | H | H | 415 | 2.88 |
| B299 | L1A | methylimino | propyl | oxo | H | difluoro- methoxy | H | H | H | H | H | H | H | H | 415 | 2.86 |
| B300 | L1A | methylimino | methyl | oxo | H | difluoro- methoxy | H | H | H | H | H | H | H | H | 387 | 2.25 |
| B301 | L1A | methylimino | 2-methylpropyl | oxo | H | difluoro- methoxy | H | H | H | H | H | H | H | H | 429 | 3.15 |
| B302 | L1A | methylimino | propan-2-yl | oxo | H | difluoro- methoxy | H | H | H | H | H | H | H | H | 415 | 2.78 |
| B303 | L1A | methylimino | 1-cyclopropyl-2-methoxyethyl | oxo | H | difluoro- methoxy | H | H | H | H | H | H | H | H | 512 | 2.94 |
| B304 | L1A | methylimino | 2-cyanoethyl | oxo | H | acetyl-amino | H | H | H | H | H | H | H | H | 417 | 2.08 |
| B305 | L1A | methylimino | propyl | oxo | H | acetyl-amino | H | H | H | H | H | H | H | H | 406 | 1.87 |
| B306 | L1A | methylimino | cyclopropylmethyl | oxo | H | acetyl-amino | H | H | H | H | H | H | H | H | 418 | 1.94 |
| B307 | L1A | methylimino | methyl | oxo | H | acetyl-amino | H | H | H | H | H | H | H | H | 378 | 1.4 |
| B308 | L1A | methylimino | 2-methylpropyl | oxo | H | acetyl-amino | H | H | H | H | H | H | H | H | 420 | 2.1 |
| B309 | L1A | methylimino | propan-2-yl | oxo | H | acetyl-amino | H | H | H | H | H | H | H | H | 406 | 1.81 |
| B310 | L1A | methylimino | 2-cyanoethyl | oxo | H | H | H | H | H | H | H | H | H | H | 360 | 2.07 |
| B311 | L1A | ethylimino | ethyl | oxo | H | H | H | H | H | H | H | H | H | H | 349 | 2.55 |
| B312 | L1A | methylimino | propyl | oxo | H | H | H | H | H | H | H | H | H | H | 349 | 2.55 |
| B313 | L1A | methylimino | cyclopropylmethyl | oxo | H | H | H | H | H | H | H | H | H | H | 361 | 2.65 |
| B314 | L1A | methylimino | methyl | oxo | H | H | H | H | H | H | H | H | H | H | 321 | 1.95 |
| B315 | L1A | methylimino | 2-methylpropyl | oxo | H | H | H | H | H | H | H | H | H | H | 363 | 2.84 |
| B316 | L1A | methylimino | ethyl | oxo | H | H | H | H | H | H | H | H | H | H | 335 | 2.2 |
| B317 | L1A | methylimino | propan-2-yl | oxo | H | H | H | H | H | H | H | H | H | H | 349 | 2.45 |
| B318 | L1A | methylimino | ethyl | thioxo | H | H | H | H | H | H | H | H | H | H | 351 | 2.73 |
| B319 | L1A | methylimino | 1-cyclopropyl-2-yl | oxo | H | H | H | H | H | H | H | H | H | H | 405 | 2.75 |
| B320 | L1A | imino | H | thioxo | H | CHF₂ | H | H | H | H | H | H | H | H | 359 | 3.05 |
| B321 | L1A | imino | H | thioxo | H | F | H | H | H | H | H | H | H | H | 327 | 2.57 |
| B322 | L1A | imino | H | thioxo | H | H | F | H | H | H | H | H | H | H | 327 | 2.47 |
| B323 | L1A | imino | propyl | oxo | H | F | H | H | H | H | H | H | H | H | 353 | 3.17 |
| B324 | L1A | imino | H | oxo | H | F | H | H | H | H | H | H | H | H | 311 | 2.09 |
| B325 | L1A | imino | prop-2-en-1-yl | oxo | H | F | H | H | H | H | H | H | H | H | 351 | 3.08 |
| B326 | L1A | ethylimino | ethyl | thioxo | H | CF₃ | H | H | H | H | H | H | H | H | 433 | 3.78 |
| B327 | L1A | ethylimino | ethyl | thioxo | H | CHF₂ | F | H | H | H | H | H | H | H | 433 | 3.37 |
| B328 | L1A | ethylimino | ethyl | thioxo | H | CHF₂ | H | H | H | H | H | H | H | H | 415 | 3.27 |
| B329 | L1A | methylimino | propan-2-yl | thioxo | H | CHF₂ | F | H | H | H | H | H | H | H | 433 | 3.29 |
| B330 | L1A | methylimino | cyclopropylmethyl | thioxo | H | Cl | H | H | H | H | H | H | H | H | 411 | 3.76 |
| B331 | L1A | methylimino | cyclopropylmethyl | thioxo | H | CHF₂ | H | H | H | H | H | H | H | H | 427 | 3.37 |
| B332 | L1A | methylimino | CH₃ | thioxo | H | CHF₂ | F | H | H | H | H | H | H | H | 405 | 2.73 |
| B333 | L1A | methylimino | ethyl | thioxo | H | CHF₂ | F | H | H | H | H | H | H | H | 419 | 3.04 |
| B334 | L1A | methylimino | propyl | thioxo | H | CHF₂ | F | H | H | H | H | H | H | H | 433 | 3.4 |
| B335 | L1A | methylimino | 2-methylpropyl | thioxo | H | CHF₂ | F | H | H | H | H | H | H | H | 447 | 3.71 |
| B336 | L1A | methylimino | propyl | thioxo | H | Cl | H | H | H | H | H | H | H | H | 399 | 3.65 |
| B337 | L1A | methylimino | cyclopropylmethyl | thioxo | H | H | H | H | H | H | H | H | H | H | 377 | 3.23 |
| B338 | L1A | methylimino | ethyl | thioxo | H | F | H | H | H | R^{a1} | H | H | H | H | 441 | 2.62 |
| B339 | L1A | methylimino | ethyl | thioxo | H | F | H | H | H | R^{a3} | H | H | H | H | 411 | 2.73 |
| B340 | L1A | methylimino | 2-cyanoethyl | thioxo | H | CHF₂ | F | H | H | H | H | H | H | H | 444 | 2.82 |
| B341 | L1A | methylimino | ethyl | thioxo | H | CHF₂ | H | H | H | H | H | H | H | H | 401 | 2.94 |
| B342 | L1A | methylimino | 2,2-difluoroethyl | thioxo | H | CHF₂ | F | H | H | H | H | H | H | H | 455 | 3.35 |
| B343 | L1A | methylimino | 2-cyanoethyl | thioxo | H | Cl | H | H | H | H | H | H | H | H | 410 | 3.02 |
| B344 | L1A | methylimino | cyclopropylmethyl | thioxo | H | H | H | H | H | R^{a3} | H | H | H | H | 419 | 3.02 |
| B345 | L1A | methylimino | ethyl | thioxo | H | F | H | H | H | R^{a2} | H | H | H | H | 407 | 3.25 |
| B346 | L1A | ethylimino | ethyl | oxo | H | CF₃ | H | H | H | H | H | H | H | H | 417 | 3.23 |
| B347 | L1A | ethylimino | methoxy | oxo | H | Cl | H | H | H | H | H | H | H | H | 384 | 2.92 |
| B348 | L1A | ethylimino | ethyl | oxo | H | 1,1,2,2- tetra-fluoro- ethoxy | H | H | H | H | H | H | H | H | 465 | 3.29 |
| B349 | L1A | ethylimino | ethyl | oxo | H | Cl | CH₃ | H | H | H | H | H | H | H | 397 | 3.44 |
| B350 | L1A | ethylimino | ethyl | oxo | F | H | H | H | H | H | H | H | H | H | 385 | 2.71 |
| B351 | L1A | ethylimino | ethyl | oxo | H | F | F | H | H | H | H | H | H | H | 385 | 2.88 |
| B352 | L1A | ethylimino | ethyl | oxo | H | CHF₂ | Cl | H | H | H | H | H | H | H | 433 | 3.23 |
| B353 | L1A | ethylimino | ethyl | oxo | H | CHF₂ | F | H | H | H | H | H | H | H | 417 | 2.86 |
| B354 | L1A | methylimino | CH₃ | oxo | F | H | H | F | H | H | H | H | H | H | 357 | 2.1 |
| B355 | L1A | methylimino | CH₃ | oxo | H | CF₃ | H | H | H | H | H | H | H | H | 389 | 2.58 |
| B356 | L1A | methylimino | propyl | oxo | H | CF₃ | H | H | H | H | H | H | H | H | 417 | 3.25 |
| B357 | L1A | methylimino | cyclopropylmethyl | oxo | H | CF₃ | H | H | H | H | H | H | H | H | 429 | 3.33 |
| B358 | L1A | methylimino | ethyl | oxo | F | H | H | F | H | H | H | H | H | H | 371 | 2.39 |
| B359 | L1A | methylimino | CH₃ | oxo | H | Cl | CH₃ | H | H | H | H | H | H | H | 369 | 2.71 |
| B360 | L1A | methylimino | propan-2-yl | oxo | H | CF₃ | H | H | H | H | H | H | H | H | 417 | 3.15 |
| B361 | L1A | methylimino | 2-methoxyethyl | oxo | H | CF₃ | H | H | H | H | H | H | H | H | 433 | 2.75 |
| B362 | L1A | methylimino | 2-methylpropyl | oxo | H | CF₃ | H | H | H | H | H | H | H | H | 431 | 3.58 |
| B363 | L1A | methylimino | 2-cyanoethyl | oxo | H | CF₃ | H | H | H | H | H | H | H | H | 428 | 2.68 |
| B364 | L1A | methylimino | CH₃ | oxo | H | 1,1,2,2- tetra-fluoro- ethoxy | H | H | H | H | H | H | H | H | 437 | 2.64 |
| B365 | L1A | methylimino | propan-2-yl | oxo | H | Cl | CH₃ | H | H | H | H | H | H | H | 397 | 3.33 |
| B366 | L1A | methylimino | 2-cyanoethyl | oxo | H | Cl | CH₃ | H | H | H | H | H | H | H | 408 | 2.8 |
| B367 | L1A | methylimino | cyclopropylmethyl | oxo | H | Cl | CH₃ | H | H | H | H | H | H | H | 409 | 3.53 |
| B368 | L1A | methylimino | 2-methylpropyl | oxo | H | Cl | CH₃ | H | H | H | H | H | H | H | 411 | 3.78 |
| B369 | L1A | methylimino | 2,2-difluoroethyl | oxo | H | cyano | H | H | H | H | H | H | H | H | 396 | 2.45 |
| B370 | L1A | methylimino | 2,2-difluoroethyl | oxo | H | CHF₂O | H | H | H | H | H | H | H | H | 437 | 2.86 |
| B371 | L1A | methylimino | 2,2-difluoroethyl | oxo | H | CHF₂ | H | H | H | H | H | H | H | H | 421 | 2.75 |
| B372 | L1A | methylimino | 2,2-difluoroethyl | oxo | H | methyl-sulfanyl | H | H | H | H | H | H | H | H | 417 | 2.96 |
| B373 | L1A | methylimino | 2,2-difluoroethyl | oxo | H | Cl | H | H | H | H | H | H | H | H | 405 | 3.06 |
| B374 | L1A | methylimino | propyl | oxo | H | Cl | CH₃ | H | H | H | H | H | H | H | 397 | 3.42 |
| B375 | L1A | methylimino | cyclopropylmethyl | oxo | H | H | H | F | H | H | H | H | H | H | 397 | 2.82 |
| B376 | L1A | methylimino | butyl | oxo | H | F | H | H | H | H | H | H | H | H | 381 | 3.09 |
| B377 | L1A | methylimino | CH₃ | oxo | H | F | F | H | H | H | H | H | H | H | 357 | 2.25 |
| B378 | L1A | methylimino | ethyl | oxo | H | F | F | H | H | H | H | H | H | H | 371 | 2.55 |
| B379 | L1A | methylimino | propyl | oxo | H | F | F | H | H | H | H | H | H | H | 385 | 2.86 |
| B380 | L1A | methylimino | 2-methylpropyl | oxo | H | F | F | H | H | H | H | H | H | H | 399 | 3.19 |
| B381 | L1A | methylimino | cyclopropylmethyl | oxo | H | F | F | H | H | H | H | H | H | H | 397 | 2.98 |
| B382 | L1A | methylimino | 2,2-difluoroethyl | oxo | H | F | F | H | H | H | H | H | H | H | 407 | 2.86 |
| B383 | L1A | methylimino | propan-2-yl | oxo | H | F | F | H | H | H | H | H | H | H | 385 | 2.78 |
| B384 | L1A | methylimino | 2-cyanoethyl | oxo | H | F | F | H | H | H | H | H | H | H | 396 | 2.35 |
| B385 | L1A | methylimino | CH₃ | oxo | H | CHF₂ | Cl | H | H | H | H | H | H | H | 405 | 2.59 |
| B386 | L1A | methylimino | ethyl | oxo | H | CHF₂ | Cl | H | H | H | H | H | H | H | 419 | 2.88 |
| B387 | L1A | methylimino | propyl | oxo | H | CHF₂ | Cl | H | H | H | H | H | H | H | 433 | 3.23 |
| B388 | L1A | methylimino | 2-methylpropyl | oxo | H | CHF₂ | Cl | H | H | H | H | H | H | H | 447 | 3.58 |
| B389 | L1A | methylimino | 2-cyanoethyl | oxo | H | CHF₂ | Cl | H | H | H | H | H | H | H | 444 | 2.69 |
| B390 | L1A | methylimino | cyclopropylmethyl | oxo | H | CHF₂ | Cl | H | H | H | H | H | H | H | 445 | 3.31 |
| B391 | L1A | methylimino | propan-2-yl | oxo | H | CHF₂ | Cl | H | H | H | H | H | H | H | 433 | 3.15 |
| B392 | L1A | methylimino | 2,2-difluoroethyl | oxo | H | CHF₂ | Cl | H | H | H | H | H | H | H | 455 | 3.19 |
| B393 | L1A | methylimino | CH₃ | oxo | H | CHF₂ | F | H | H | H | H | H | H | H | 389 | 2.25 |
| B394 | L1A | methylimino | ethyl | oxo | H | CHF₂ | F | H | H | H | H | H | H | H | 403 | 2.55 |
| B395 | L1A | methylimino | 2-methylpropyl | oxo | H | CHF₂ | F | H | H | H | H | H | H | H | 431 | 3.15 |
| B396 | L1A | methylimino | propan-2-yl | oxo | H | CHF₂ | F | H | H | H | H | H | H | H | 417 | 2.78 |
| B397 | L1A | methylimino | 2-cyanoethyl | oxo | H | CHF₂ | F | H | H | H | H | H | H | H | 428 | 2.37 |
| B398 | L1A | methylimino | 2,2-difluoroethyl | oxo | H | CHF₂ | F | H | H | H | H | H | H | H | 439 | 2.84 |
| B399 | L1A | methylimino | propyl | oxo | H | CHF₂ | F | H | H | H | H | H | H | H | 417 | 2.86 |
| B400 | L1A | methylimino | ethyl | oxo | H | CHF₂ | H | H | H | H | H | H | H | H | 385 | 2.44 |
| B401 | L1A | methylimino | cyclopropylmethyl | oxo | H | H | H | H | H | R^{a1} | H | H | H | H | 433 | 2.37 |
| B402 | L1A | methylimino | cyclopropylmethyl | oxo | H | H | H | H | H | R^{a2} | H | H | H | H | 399 | 2.98 |
| B403 | L1A | methylimino | 2-cyanoethyl | oxo | H | Cl | H | H | H | R^{a2} | H | H | H | H | 432 | 2.77 |
| B404 | L1A | oxo | H | oxo | H | Cl | H | H | H | H | H | H | H | H | 328 | 2.11 |
| B405 | L1A | imino | H | oxo | H | CHF₂ | H | H | H | H | H | H | H | H | 343 | 2.04^{[c]} |
| B406 | L1A | oxo | CH₃ | oxo | H | CHF₂ | H | H | H | H | H | H | H | H | 358 | 2.55 |
| B407 | L1A | oxo | H | oxo | H | H | H | CHF₂ | H | H | H | H | H | H | 344 | 1.98 |
| B409 | L1A | methylimino | ethyl | oxo | H | H | H | CHF₂O | H | H | H | H | H | H | 401 | 2.57 |
| B410 | L1A | methylimino | cyclopropylmethyl | oxo | H | CHF₂O | H | H | H | H | H | H | H | H | 427 | 2.98 |
| B411 | L1A | methylimino | cyclopropylmethyl | oxo | H | H | F | CHF₂ | H | H | H | H | H | H | 429 | 2.98 |
| B412 | L1A | methylimino | methoxy | oxo | H | H | H | CHF₂ | H | H | H | H | H | H | 387 | 2.34 |
| B413 | L1A | ethylimino | methoxy | oxo | H | CHF₂ | H | H | H | H | H | H | H | H | 401 | 2.64 |
| B414 | L1A | ethylimino | ethoxy | oxo | H | CHF₂ | H | H | H | H | H | H | H | H | 415 | 2.88 |
| B415 | L1A | methylimino | ethoxy | oxo | H | CHF₂ | H | H | H | H | H | H | H | H | 401 | 2.57 |
| B416 | L1A | oxo | CH₃ | oxo | H | methoxy | F | H | H | H | H | H | H | H | 356 | 2.43 |
| B417 | L1A | oxo | CH₃ | oxo | H | methoxy | Cl | H | H | H | H | H | H | H | 372 | 2.80 |
| B418 | L1A | oxo | CH₃ | oxo | H | F | methoxy | H | H | H | H | H | H | H | 356 | 2.37 |
| B419 | L1A | oxo | CH₃ | oxo | H | H | CH₃ | Cl | H | H | H | H | H | H | 356 | 3.17 |
| B420 | L1A | ethylimino | prop-2-yn-1-yl | oxo | H | CHF₂ | H | H | H | H | H | H | H | H | 409 | 2.86 |
| B421 | L1A | oxo | CH₃ | oxo | H | methoxy | bromo | H | H | H | H | H | H | H | 416 | 2.90 |
| B422 | L1A | oxo | CH₃ | oxo | H | methoxy | methoxy | H | H | H | H | H | H | H | 368 | 2.02^{[c]} |
| B423 | L1A | methylimino | CH₃ | thioxo | H | H | H | CHF₂ | H | H | H | H | H | H | 387 | 2.66 |
| B424 | L1A | methylimino | ethyl | thioxo | H | CHF₂O | H | H | H | H | H | H | H | H | 417 | 3.06 |

Abbreviations: R^{a1} = methoxyacetyl; R^{a2} = prop-2-yn-1-yl; R^{a3} = acetyl 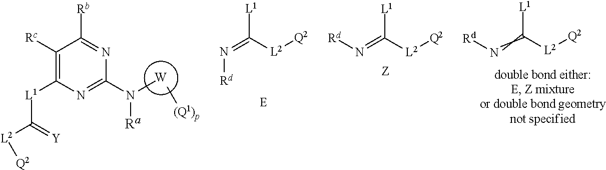
The double bond geometry of C═Y of the following compounds of table II is double bond either (a mixture of E, Z-isomers or double bond geometry not specified):
B29, B30, B31, B32, B34, B35, B36, B37, B38, B39, B40, B41, B42, B43, B44, B46, B47, B48, B49, B50, B53, B54, B55, B58, B60, B64, B65, B68, B69, B70, B72, B73, B74, B79, B80, B84, B85.
The double bond geometry of C═Y of the following compounds of table II is E:
B104, B106, B107, B108, B109, B110, B111, B27, B75, B76, B77, B78, B81, B82, B83, B90.
Of course all stereoisomeric forms (E, Z, mixture of E,Z = double bond either) are claimed in this invention.
Measurement of logP values was performed according EEC directive 79/831 Annex V.A8 by HPLC (High Performance Liquid Chromatography) on reversed phase columns with the following methods:
Usually measurement of LC-MS was done at pH 2.7 with 0.1% formic acid in water and with acetonitrile (contains 0.1% formic acid) as eluent with a linear gradient from 10% acetonitrle to 95% acetonitrile.
^{[b]}Measurement was done at pH 2.3 with 0.1% phosphoric acid and acetonitrile as eluent.
^{[c]}Measurement with LC-MS was done at pH 7.8 with 0.001 molar ammonium hydrogen carbonate solution in water as eluent with a linear gradient from 10% acetonitrile to 95 % acetonitrile.
Calibration was done with not branched alkan2-ones (with 3 to 16 carbon atoms) with known logP-values (measurement of logP values using retention times with linear interpolation between successive alkanones).. lambda-maX-values were determined using UV-spectra from 200 nm to 400 nm and the peak values of the chromatographic signals.

TABLE III
Formula (III₁)

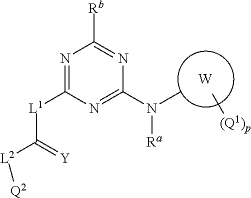

| | In table III we use the same abbreviations | |
|---|---|---|
| | for *—L¹—C(═Y)—L²—Q² as in table I. | |
| Formula (IV) | *—L¹—C(═Y)—L²—Q² | Abbreviation |
| 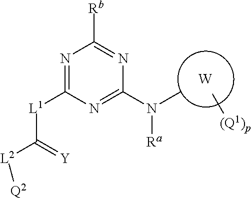 Formula (III₁) Whereas W represents a saturated or unsaturated, aromatic or non-aromatic 4-, 5-, 6- or 7-membered heterocycle comprising up to four heteroatoms which may be the same or different | 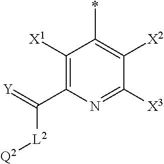 | L1A |

| | | | | | | | | | | | Meas- | |
|---|---|---|---|---|---|---|---|---|---|---|---|---|
| Example | | | | | | | | | | | ured | |
| number | L¹C(═Y)L²Q² | L² | Q² | Y | HET-(Q1)p | X¹ | X² | X³ | R^{a} | R^{b} | MW | log p |
| C1 | L1A | bond | pyrrolidin-1-yl | oxo | 6-methoxypyridin-3-yl | H | H | H | H | H | 378 | 1.99^{[c]} |
| C2 | L1A | bond | piperidin-1-yl | oxo | 6-methoxypyridin-3-yl | H | H | H | H | H | 392 | 2.14^{[c]} |
| C3 | L1A | propylimino | propyl | oxo | 6-methoxypyridin-3-yl | H | H | H | H | H | 408 | 2.77^{[c]} |
| C4 | L1A | ethylimino | ethyl | oxo | 6-methoxypyridin-3-yl | H | H | H | H | H | 380 | 2.11^{[c]} |
| C5 | L1A | methylimino | propyl | oxo | 6-methoxypyridin-3-yl | H | H | H | H | H | 380 | 2.11^{[c]} |
| C6 | L1A | methylimino | cyclopropyl- | oxo | 6-methoxypyridin-3-yl | H | H | H | H | H | 392 | 2.2 |
| | | | methyl | | | | | | | | | |
| C7 | L1A | prop-2-en-1- | prop-2-en-1-yl | oxo | 6-methoxypyridin-3-yl | H | H | H | H | H | 404 | 2.58^{[c]} |
| | | ylimino | | | | | | | | | | |
| C8 | L1A | methylimino | methyl | oxo | 6-methoxypyridin-3-yl | H | H | H | H | H | 352 | 1.57 |
| C9 | L1A | methylimino | 2-methylpropyl | oxo | 6-methoxypyridin-3-yl | H | H | H | H | H | 394 | 2.37 |
| C10 | L1A | methylimino | ethyl | oxo | 6-methoxypyridin-3-yl | H | H | H | H | H | 366 | 1.82 |
| C11 | L1A | ethylimino | butyl | oxo | 6-methoxypyridin-3-yl | H | H | H | H | H | 408 | 2.78 |
| C12 | L1A | methylimino | 2-cyanoethyl | oxo | thiophen-3-yl | H | H | H | H | H | 366 | 1.89 |
| C13 | L1A | ethylimino | ethyl | oxo | thiophen-3-yl | H | H | H | H | H | 355 | 2.44 |
| C14 | L1A | methylimino | propyl | oxo | thiophen-3-yl | H | H | H | H | H | 355 | 2.45 |
| C15 | L1A | methylimino | cyclopropyl- | oxo | thiophen-3-yl | H | H | H | H | H | 367 | 2.53 |
| | | | methyl | | | | | | | | | |
| C16 | L1A | methylimino | 2-methylpropyl | oxo | thiophen-3-yl | H | H | H | H | H | 369 | 2.63 |
| C17 | L1A | methylimino | ethyl | oxo | thiophen-3-yl | H | H | H | H | H | 341 | 2.13 |
| C18 | L1A | methylimino | 2-methylpropyl | oxo | pyridin-4-yl | H | H | H | H | H | 364 | 1.19 |
| C19 | L1A | methylimino | CH₃ | oxo | thiophen-3-yl | H | H | H | H | H | 327 | 1.86 |
| C20 | L1A | methylimino | propan-2-yl | oxo | thiophen-3-yl | H | H | H | H | H | 355 | 2.37 |
| C21 | L1A | methylimino | 2,2-difluoroethyl | oxo | thiophen-3-yl | H | H | H | H | H | 377 | 2.46 |
| C22 | L1A | ethylimino | ethyl | S | thiophen-3-yl | H | H | H | H | H | 371 | 3.02 |
| C23 | L1A | methylimino | ethyl | S | thiophen-3-yl | H | H | H | H | H | 357 | 2.68 |
| C24 | L1A | O | CH₃ | oxo | thiophen-3-yl | H | H | H | H | H | 314 | 2.23 |
| C25 | L1A | O | CH₃ | oxo | 2-chloropyridin-4-yl | H | H | H | H | H | 343 | 2.02 |
| C28 | L1A | O | H | oxo | thiophen-3-yl | H | H | H | H | H | 300 | 1.62 |
| C29 | L1A | methylimino | 2-cyanoethyl | oxo | thiophen-3-yl | H | H | H | CH₃—CO | H | 408 | 1.78 |
| C30 | L1A | methylimino | methoxy | oxo | thiophen-3-yl | H | H | H | H | H | 343 | 2.05 |
| C31 | L1A | ethylimino | ethoxy | oxo | thiophen-3-yl | H | H | H | H | H | 371 | 2.59 |
| C32 | L1A | ethylimino | methoxy | oxo | thiophen-3-yl | H | H | H | H | H | 357 | 2.33^{[c]} |
| C33 | L1A | methylimino | ethoxy | oxo | thiophen-3-yl | H | H | H | H | H | 357 | 2.26 |

Measurement of logP values was performed according EEC directive 79/831 Annex V.A8 by HPLC (High Performance Liquid Chromatography) on reversed phase columns with the following methods:
Usually measurement of LC-MS was done at pH 2.7 with 0.1% formic acid in water and with acetonitrile (contains 0.1% formic acid) as eluent with a linear gradient from 10% acetonitrle to 95% acetonitrile.
^{[b]}Measurement was done at pH 2.3 with 0.1% phosphoric acid and acetonitrile as eluent.
^{[c]}Measurement with LC-MS was done at pH 7.8 with 0.001 molar ammonium hydrogen carbonate solution in water as eluent with a linear gradient from 10% acetonitrile to 95% acetonitrile.
Calibration was done with not branched alkan2-ones (with 3 to 16 carbon atoms) with known logP-values (measurement of logP values using retention times with linear interpolation between successive alkanones). lambda-maX-values were determined using UV-spectra from 200 nm to 400 nm and the peak values of the chromatographic signals.

The following examples illustrate in a non-limiting manner the preparation and efficacy of the compounds of formulas (II) to (IV) according to the invention.

### Preparation of ethyl 4-{2-[(6-methoxypyridin-3-yl)amino]pyrimidin-4-yl}puridine-2-carboxylate (Compound A-1)

150 mg of ethyl 3-[3-(dimethylamino)prop-2-enoyl]pyridine-2-carboxylate (0.6 mmol), 138 mg of 1-(6-methoxypyridin-3-yl)guanidine nitrate (0.6 mmol) and 64 mg of sodium carbonate (0.6 mmol) were stirred for 8 hours at reflux in 3.38 ml of 2-methoxyethanol. After cooling water was added and the precipitate was filtered and dried to yield 65 mg of ethyl 4-{2-[(6-methoxypyridin-3-yl)amino]-pyrimidin-4-yl}pyridine-2-carboxylate (yield=53%) [M+1]=352.

### Preparation of 4-{2-[(2-chloropyridin-4-yl)amino]pyrimidin-4-yl}-N-ethyl-N-methylpyridine-2-carboxamide (Compound A-17) and N-ethyl-4-[2-({2-[ethyl(methyl)carbamoyl]pyridin-4-yl}amino)pyrimidin-4-yl]-N-methylpyridine-2-carboxamide (Compound A-19) according to process P1A

### Step 1: Preparation of di-tert-butyl {(Z)-[(2-chloropyridin-4-yl)amino]methylylidene}biscarbamate

20.22 g (0.157 mol) of 4-amino-2-chloropyridine were diluted in triethylamine (67 ml) and dichloromethane (600 ml) at 0-5° C. 47 g of Mercury(II) chloride (0.173 mol) and 50.24 g (0.173 mol) of N,N′-bis(boc)-S-methyl-isothiourea were added to the reaction mixture, which was then stirred at room temperature for 4 days, filtered on a fritted funnel, concentrated in vacuo and chromatographed on silica (Heptane90/AcOEt10) to yield 43.67 g of di-tert-butyl {(Z)-[(2-chloropyridin-4-yl)amino]methylylidene}biscarbamate (yield=71%). [M+1]=371

### Step 2: Preparation of 1-(2-chloropyridin-4-yl)guanidine bis(trifluoroacetate)

To a solution of 43.67 g (0.117 mol) of di-tert-butyl {(Z)-[(2-chloropyridin-4-yl)amino]methylylidene}biscarbamate in dichloromethane (800 ml) at room temperature were added 81.64 ml of trifluoroacetic acid (1.06 mol). The reaction mixture was stirred at room temperature for 2 days, concentrated in vacuo, triturated with 100 ml of pentane, and upon standing crystallized to yield 51.68 g of 1-(2-chloropyridin-4-yl)guanidine bis(trifluoroacetate) (yield=99%). [M+1-2*CF₃CO₂H]=171

### Step 3: Preparation of N,4-bis(2-chloropyridin-4-yl)pyrimidin-2-amine according to process A-1

To a solution of 6.32 g of 1-(2-chloropyridin-4-yl)-3-(dimethylamino)prop-2-en-1-one (30 mmol) in 60 ml of 2-Propanol was added 2.52 g of sodium hydroxide (63 mmol) and 11.96 g of 1-(2-chloropyridin-4-yl)guanidine bis(trifluoroacetate) (30 mmol). The reaction mixture was heated to reflux under stirring for 20 h. After filtration, the precipitate was washed with 100 ml of n-butanol and 120 ml of i-Pr₂O and then air-dried to yield 4.69 g of N,4-bis(2-chloropyridin-4-yl)pyrimidin-2-amine (yield=37%). [M+1]=318

### Step 4: Preparation of 4-{2-[(2-chloropyridin-4-yl)amino]pyrimidin-4-yl}-N-ethyl-N-methylpyridine-2-carboxamide (Compound A-17) and N-ethyl-4-[2-({2-[ethyl(methyl)carbamoyl]pyridin-4-yl}amino)pyrimidin-4-yl]-N-methylpyridine-2-carboxamide (Compound A-19)

200 mg of N,4-bis(2-chloropyridin-4-yl)pyrimidin-2-amine (0.63 mmol), 111 mg of N-Methyl-N-ethylamine (1.89 mmol), 166 mg of molybdenum hexacarbonyl (0.631 mmol), 0.282 ml of 1,8-Diazabicyclo(5.4.0)undec-7-ene (1.89 mmol) and 72.9 mg (0.063 mmol) of Tetrakis(triphenylphosphine)palladium(0) were diluted in 5 ml of N,N-dimethylformamide. The reaction mixture was stirred at 100° C. for 5 hours. After cooling, 10 ml of a saturated NH₄Cl was added and the mixture was extracted with 5 ml of dichloromethane. After evaporation of the solvent the crude product was chromatographed on silica (dichloromethane/ethanol) to yield 58 mg of 4-{2-[(2-chloropyridin-4-yl)amino]pyrimidin-4-yl}-N-ethyl-N-methylpyridine-2-carboxamide (yield=22%) [M+1]=479 and 130 mg of N-ethyl-4-[2-({2-[ethyl(methyl)carbamoyl]pyridin-4-yl}amino)pyrimidin-4-yl]-N-methylpyridine-2-carboxamide (yield=44%) [M+1]=420.

### Preparation of 4-{2-[(5-tert-butyl-2-thienyl)amino]pyrimidin-4-yl}-N,N-diethylpyridine-2-carboxamide (compound A-39) according to process P12

### Step 1: Preparation of N,N-diethyl-4-iodopyridine-2-carboxamide

To a solution of 24.5 g (93.4 mmol) 4-iodopyridine-2-carboxylic acid in 600 ml dichloromethane was added under an argon atmosphere 34 ml (197 mmol) N,N-diisopropylethylamine. After cooling to 0° C. 12.7 ml (103 mmol) 2,2-dimethylpropionylchloride was added dropwise and stirred for 1 h at ° C. followed by the dropwise addition of 20.3 ml (197 mmol) diethylamine. Stirring was continued for 1 hour at 0° C. and 1 hour at room temperature. Then 300 ml water was added and the aqueous phase extracted additional two times with 100 ml dichloromethane. The combined organic layers were dried and the solvent was removed in vacuo. The crude product was purified by chromatography on silica (heptane/ethyl acetate) to yield 23.4 g N,N-diethyl-4-iodopyridine-2-carboxamide (yield=72%) [M+1]=305.

### Step 2: Preparation of N,N-diethyl-4-(tributylstannyl)pyridine-2-carboxamide

5 g (16.44 mmol) of N,N-diethyl-4-iodopyridine-2-carboxamide (obtained from step 1) was dissolved under an argon atmosphere in 40 ml of 1,4-dioxane followed by the addition of 19.075 g (32.88 mmol) hexabutylditin and 0.577 g (0.822 mmol) dichlorobis(triphenylphosphine)palladium(II). The mixture was refluxed for 3 hours. After cooling the suspension was passed through a 10 g silica cartridge, the cartridge was rinsed with 10 ml 1,4-dioxane and the solvent was removed in vacuo. The crude product was purified by chromatography on silica (heptane/ethyl acetate) to yield 4.27 g of N,N-diethyl-4-(tributylstannyl)pyridine-2-carboxamide (yield=55%) [M+1]=468.

### Step 3: Preparation of 4-(2-chloropyrimidin-4-yl)-N,N-diethylpyridine-2-carboxamide

A solution of 2.5 g (5.35 mmol) N,N-diethyl-4-(tributylstannyl)pyridine-2-carboxamide (obtained from step 2) in 18 ml 1,4-dioxane was placed in a 20 ml microwave tube followed by the addition of 1.03 g (7 mmol) 2,4-dichloropyrimidine and 0.62 g (0.535 mmol) Tetrakis(triphenylphosphine)palladium(0). The mixture was microwaved in a Biotage Optimizer at 150° C. for 20 minutes. After cooling 20 ml of dichloromethane was added, the resulting suspension was filtrated and the filtrate was concentrated in vacuo. To the obtained residue was added 50 ml of saturated potassium fluoride solution and stirred for 15 min followed by the extraction with ethyl acetate. The combined organic layers were dried, evaporated and the crude product was purified by chromatography on silica (heptane/ethyl acetate) followed by chromatography on silica (dichloromethane/acetone) to yield 400 mg 4-(2-chloropyrimidin-4-yl)-N,N-diethylpyridine-2-carboxamide (yield=25%). [M+1]=291.

### Step 4: Preparation of tert-butyl (5-tert-butyl-2-thienyl)carbamate

Under an argon atmosphere 1 g (5.4 mmol) 5-tert-butylthiophene-2-carboxylic acid was dissolved in 10 ml of tert-butanol. After the addition of 1.54 g (5.4 mmol) diphenylphosphoroazidate and of 0.76 ml (5.4 mmol) triethylamine the resulting mixture was refluxed for 6 hours followed by stirring at 50° C. for 16 hours. After cooling 50 ml of water was added and the mixture was extracted 3 times with 10 ml of ethyl acetate. The combined organic layers were dried and after evaporation of the solvent the crude product was purified by chromatography on silica (heptane/ethyl acetate) to yield 415 mg of tert-butyl (5-tert-butyl-2-thienyl)carbamate (yield=30%).

### Step 5: Preparation of 4-{2-[(5-tert-butyl-2-thienyl)amino]pyrimidin-4-yl}-N,N-diethylpyridine-2-carboxamide (compound A-39)

To a solution of 105 mg (0.41 mmol) tert-butyl (5-tert-butyl-2-thienyl)carbamate (obtained from step 4) in 3 ml of 1,4-dioxane were added 100 mg (0.34 mmol) 4-(2-chloropyrimidin-4-yl)-N,N-diethylpyridine-2-carboxamide (obtained from step 3) and 98 mg (0.51 mmol) 4-toluenesulfonic acid monohydrate and refluxed for 20 hourss. After cooling 3 ml of water was added and the mixture was extracted 3 times with 3 ml of dichloromethane. The combined organic layers were dried and after evaporation of the solvent the crude product was purified by chromatography on silica to yield 60 mg of 4-{2-[(5-tert-butyl-2-thienyl)amino]pyrimidin-4-yl}-N,N-diethylpyridine-2-carboxamide (yield=41%) [M+1]=410.

### Preparation of N,N-diethyl-4-[5-methyl-2-(3-thienylamino)pyrimidin-4-yl]pyridine-2-carboxamide (compound A-41) according to process P12

### Step 1: Preparation of 4-(2-chloro-5-methylpyrimidin-4-yl)-N,N-diethylpyridine-2-carboxamide

A solution of 1 g (2.05 mmol) N,N-diethyl-4-(tributylstannyl)pyridine-2-carboxamide (obtained from step 2 compound A-39) in 8 ml 1,4-dioxane was placed in a 10 ml microwave tube followed by the addition of 435 mg (2.67 mmol) 2,4-dichloro-5-methylpyrimidine and 0.23 g (0.2 mmol) Tetrakis(triphenylphosphine)palladium(0). The mixture was microwaved in a Biotage Optimizer at 150° C. for 20 minutes. After cooling 10 ml of dichloromethane was added, the resulting suspension was filtrated and the filtrate was concentrated in vacuo. To the obtained residue was added 20 ml of saturated potassium fluoride solution and stirred for 15 min followed by the extraction with ethyl acetate. The combined organic layers were dried, evaporated and the crude product was purified by chromatography on silica (heptane/ethyl acetate) to yield 400 mg of 4-(2-chloro-5-methylpyrimidin-4-yl)-N,N-diethylpyridine-2-carboxamide (yield=30%) [M+1]=306.

### Step 2: Preparation of N,N-diethyl-4-[5-methyl-2-(3-thienylamino)pyrimidin-4-yl]pyridine-2-carboxamide (compound A-41)

To a solution of 60 mg (0.2 mmol) 4-(2-chloro-5-methylpyrimidin-4-yl)-N,N-diethylpyridine-2-carboxamide (obtained from step 1) in 3 ml of 1,4-dioxane were added 144 mg (0.76 mmol) thiophen-3-ylamine oxalic acid salt and 21 mg (0.11 mmol) 4-toluenesulfonic acid monohydrate and refluxed for 10 days. After cooling 3 ml of water was added and the mixture was extracted 3 times with 3 ml of dichloromethane. The combined organic layers were washed succesively with 2 ml of 1M NaOH, 2 ml of 1M HCl and brine. After evaporation of the solvent the crude product was purified by prep hplc to yield 3 mg of N,N-diethyl-4-[5-methyl-2-(3-thienylamino)pyrimidin-4-yl]pyridine-2-carboxamide (yield=4%) [M+1]=368.

### Preparation of 1-(4-{4-[(3-chlorophenyl)amino]-1,3,5-triazin-2-yl}pyridin-2-yl)ethanone (Compound B-26) according to process P1

### Step 1

### Preparation of 4-{4-[(3-chlorophenyl)amino]-1,3,5-triazin-2-yl}pyridine-2-carbonitrile

To a solution of 70 g of N-(3-chlorophenyl)-4-(2-chloropyridin-4-yl)-1,3,5-triazin-2-amine (prepared as described in WO 2005/033095) in 350 ml of N,N-dimethylformamide were added under argon 38.75 g (330 mmol) zink cyanide and 50.85 g (44 mmol) Tetrakis(triphenylphosphine)palladium(0). The mixture was heated for 3 hours at 100° C. After cooling the resulting slurry was filtered, the precipitate was washed with N,N-dimethylformamide and the combined filtrates were evaporated. The remaining solid was recrystallized from dichloromethane yielding 54.39 g of 4-{4-[(3-chlorophenyl)amino]-1,3,5-triazin-2-yl}pyridine-2-carbonitrile (yield=80%) [M+1]=310.

### Step 2

To 130 ml of tetrahydrofuran was added 26 ml of a 3 M solution of methylmagnesium bromide in toluene and cooled to 0° C. Then 8 g (26 mmol) of 4-{4-[(3-chlorophenyl)amino]-1,3,5-triazin-2-yl}pyridine-2-carbonitrile was added in small portions and stirring was continued for 3 hours at 0° C. After warming to room temperature stirring was continued for 4 hours. Then 120 ml of 1N HCl was added and the mixture was extracted with ethyl acetate. The combined organic phases were dried and evaporated to yield 8.13 g of 1-(4-{4-[(3-chlorophenyl)amino]-1,3,5-triazin-2-yl}pyridin-2-yl)ethanone (yield=96%) [M+1]=327.

### Preparation of N-(3-chlorophenyl)-4-{2-[(1E)-N-methoxyethanimidoyl]pyridin-4-yl}-1,3,5-triazin-2-amine (Compound B-27) according to process P2

50 mg (0.15 mmol) of 1-(4-{4-[(3-chlorophenyl)amino]-1,3,5-triazin-2-yl}pyridin-2-yl)ethanone, 26 mg (0.3 mmol) of O-methylhydroxylamine hydrochloride and 26 mg (0.31 mmol) sodium acetate dissolved in 3 ml ethanol were stirred at reflux for 6 hours. After cooling the solvent was evaporated in vacuo and 5 ml of water was added. The solid was filtrated, washed with water and dried to yield 21 mg of N-(3-chlorophenyl)-4-{2-[(1E)-N-methoxyethanimidoyl]pyridin-4-yl}-1,3,5-triazin-2-amine (yield=35%) [M+1]=356.

### Preparation of 3-{2-[1-(4-{4-[(3-chlorophenyl)amino-1-1,3,5-triazin-2-yl}pyridin-2-yl)ethylidene]hydrazinyl}propanenitrile (Compound B-81) according to process P2

To a solution of 200 mg (0.62 mmol) 1-(4-{4-[(3-chlorophenyl)amino]-1,3,5-triazin-2-yl}pyridin-2-yl)ethanone, 0.028 ml (0.49 mmol) acetic acid and 51 mg (0.62 mmol) sodium acetate in 2 ml methanol was added 52 mg (0.62 mmol) of 3-hydrazinylpropanenitrile dissolved in 2 ml of methanol and stirred at reflux for 2 hours and at room temperature overnight. The solvent was evaporated in vacuo and 30 ml of water was added. The mixture was extracted with dichloromethane, the combined organic phases were dried and evaporated to yield 1280 mg of 3-{2-[1-(4-{4-[(3-chlorophenyl)amino]-1,3,5-triazin-2-yl}pyridin-2-yl)ethylidene]hydrazinyl}propanenitrile (yield=53%) [M+1]=394.

### Preparation of 4-{4-[(3-chlorophenyl)amino]-1,3,5-triazin-2-yl}-N′-hydroxypyridine-2-carboximidamide (Compound B-78) according to process P1

A solution of 1.5 g (4.86 mmol) of 4-{4-[(3-chlorophenyl)amino]-1,3,5-triazin-2-yl}pyridine-2-carbonitrile, 675 mg (9.7 mmol) hydroxylamine hydrochloride and 1.34 g (9.7 mmol) potassium carbonate dissolved in 20 ml ethanol was stirred at room temperature for 5 hours. After evaporation of the solvent water was added and the remaining solid was filtered and dried to yield 1.566 g of 4-{4-[(3-chlorophenyl)amino]-1,3,5-triazin-2-yl}-N′-hydroxypyridine-2-carboximidamide (yield=94%) [M+1]=343.

### Preparation of 4-{4-[(3-chlorophenyl)amino]-1,3,5-triazin-2-yl}-N′-[(methoxyacetyl)oxy]pyridine-2-carboximidamide (Compound B-104)

A solution of 200 mg (0.58 mmol) of 4-{4-[(3-chlorophenyl)amino]-1,3,5-triazin-2-yl}-N′-hydroxypyridine-2-carboximidamide, 63 mg (0.58 mmol) methoxyacetyl chloride and 49 mg (0.58 mmol) sodium bicarbonate dissolved in 5 ml acetone was stirred at room temperature for 16 hours. After evaporation of the solvent 30 ml of water was added and the remaining solid was filtered and dried to yield 145 mg of 4-{4-[(3-chlorophenyl)amino]-1,3,5-triazin-2-yl}-N′-[(methoxyacetyl)oxy]pyridine-2-carboximidamide (yield=60%) [M+1]=415.

### Preparation of ethyl 4-{4-[(3-chlorophenyl)amino]-1,3,5-triazin-2-yl}pyridine-2-carboximidoate (Compound B-102) according to process P1

A solution of 200 mg (0.65 mmol) 4-{4-[(3-chlorophenyl)amino]-1,3,5-triazin-2-yl}pyridine-2-carbonitrile, 126 mg (1.3 mmol) O-ethylhydroxylamine hydrochloride and 179 mg 1.3 mmol) potassium carbonate dissolved in 20 ml ethanol was stirred at 70° C. for 5 hours. After cooling and evaporation of the solvent water was added and the remaining solid was filtered and dried to yield 178 mg of 4-{4-[(3-chlorophenyl)amino]-1,3,5-triazin-2-yl}pyridine-2-carboximidoate (yield=74%) [M+1]=356.

### Preparation of 4-{4-[(3-chlorophenyl)amino]-1,3,5-triazin-2-yl}-N-hydroxypyridine-2-carboxamide (Compound B-185) according to process P9

### Step 1: Preparation of 4-{4-[(3-chlorophenyl)amino]-1,3,5-triazin-2-yl}pyridine-2-carboxylic acid

To a solution of 3 g (9.7 mmol) of 4-{4-[(3-chlorophenyl)amino]-1,3,5-triazin-2-yl}pyridine-2-carbonitrile in 60 ml of ethanol was added 1.166 g (29.1 mmol) sodium hydroxide dissolved in 35 ml water. The mixture was heated at reflux for 2 hours. After cooling water was added and the mixture acidified with 1 N HCl. The precipitate which formed was filtered, washed with water and dried to yield 2.6 g of 4-{4-[(3-chlorophenyl)amino]-1,3,5-triazin-2-yl}pyridine-2-carboxylic acid (yield=81%) [M+1]=329

### Step 2: Preparation of 4-{4-[(3-chlorophenyl)amino]-1,3,5-triazin-2-yl}-N-hydroxypyridine-2-carboxamide

To a solution of 500 mg (1.53 mmol) of 4-{4-[(3-chlorophenyl)amino]-1,3,5-triazin-2-yl}pyridine-2-carboxylic acid dissolved in 6 ml of dimethylformamide were added 463 mg (4.58 mmol) triethylamine, 212 mg (3.05 mmol) hydroxylamine hydrochloride and 696 mg (1.83 mmol) O-(7-Azabenzotriazol-1-yl)-N,N,N′,N′-tetra-methyluronium hexafluorophosphate. The mixture was stirred at room temperature for 16 hours. After addition of water the mixture was extracted with dichloromethane and the organic layer was washed successively with 1 N HCl, saturated sodium bicarbonate and saturated lithium chloride solution. After evaporation of the solvent the residue was stirred with methanol, filtered and dried to yield 165 mg of 4-{4-[(3-chlorophenyl)amino]-1,3,5-triazin-2-yl}-N-hydroxypyridine-2-carboxamide (yield=29%) [M+1]=344.

### Preparation of N-ethyl-N-methyl-4-(4-{[3-(pentafluoro-lambda⁶-sulfanyl)phenyl]amino}-1,3,5-triazin-2-yl)pyridine-2-carbothioamide (Compound B-253) according to process P10

To 130 mg (0.28 mmol) of N-ethyl-N-methyl-4-(4-{[3-(pentafluoro-lambda⁶-sulfanyl)phenyl]amino}-1,3,5-triazin-2-yl)pyridine-2-carboxamide dissolved in 5 ml of toluene was added 31.4 mg (0.14 mmol) of phosphorous pentasulfide. The reaction mixture was stirred under reflux for two hours, then 2 ml of water were added and the reaction mixture was stirred at 100° C. for one hour. After cooling, water was removed by filtration on solid-phase extraction cartridge. After rinsing of the cartridge with 3 ml of toluene, the filtrate was purified by chromatography on silica (dichloromethane/ethanol) to yield 55 mg of N-ethyl-N-methyl-4-(4-{[3-(pentafluoro-lambda⁶-sulfanyl)phenyl]amino}-1,3,5-triazin-2-yl)pyridine-2-carbothioamide (yield=41%) [M+1]=477.

### Preparation of N-(4-{2-[(cyclopropylmethyl)(methyl)carbamothioyl]pyridin-4-yl}-1,3,5-triazin-2-yl)-N-phenylacetamide (compound B-344)

118 mg (0.71 mmol) of lithium hexamethyldisilazane were added to 263 mg (0.64 mmol) of 4-(4-anilino-1,3,5-triazin-2-yl)-N-(cyclopropylmethyl)-N-methylpyridine-2-carbothioamide (prepared analogously to compound B-253) dissolved in 5 ml anhydrous tetrahydrofuran. After stirring for 30 minutes 55 mg (0.71 mmol) of acetyl chloride was added and stirring continued for 20 hours. 5 ml of water were then added and the organic phase is filtered through a Chemelute cartridge. Dichloromethane ifollowed by ethyl acetate were used to rinse the cartridge and the concentrated organic phases are purified on a silica gel column (ethyl acetate/dichloromethane) followed by prep rp-hplc to give 110 mg of N-(4-{2-[(cyclopropylmethyl)(methyl)carbamothioyl]pyridin-4-yl}-1,3,5-triazin-2-yl)-N-phenylacetamide (yield=39%) [M+1]=419.

### Preparation of N-(cyclopropylmethyl)-N-methyl-4-{4-[phenyl(prop-2-yn-1-yl)amino]-1,3,5-triazin-2-yl}pyridine-2-carboxamide (compound B-402)

371 mg (2.22 mmol) of lithium hexamethyldisilazane were added to 400 mg (1.11 mmol) of 4-(4-anilino-1,3,5-triazin-2-yl)-N-(cyclopropylmethyl)-N-methylpyridine-2-carboxamide (prepared analogously to compound C-2) dissolved in 5 ml anhydrous tetrahydrofuran. After stirring for 30 minutes 330 mg (2.22 mmol) of 3-bromoprop-1-yne was added and stirring continued for 20 hours. 4 ml of water were then added and the organic phase is filtered through a Chemelute cartridge. Dichloromethane is used to rinse the cartridge and the concentrated organic phases are purified on a silica gel column (ethyl acetate/dichloromethane) to give 180 mg of N-(cyclopropylmethyl)-N-methyl-4-{4-[phenyl(prop-2-yn-1-yl)amino]-1,3,5-triazin-2-yl}pyridine-2-carboxamide (yield=26%) [M+1]=399.

### Preparation of (4-{4-[(6-methoxypyridin-3-yl)amino]-1,3,5-triazin-2-yl}pyridin-2-yl)(piperidin-1-yl)methanone (Compound C-2) according to process P1

### Step 1: Preparation of 4-(2-chloropyridin-4-yl)-N-(6-methoxypyridin-3-yl)-1,3,5-triazin-2-amine

To a solution of 3.406 g (15 mmol) of 2-chloro-4-(2-chloropyridin-4-yl)-1,3,5-triazine (prepared as described in WO 2001/25220) and of 1.862 g (15 mmol) 6-methoxypyridin-3-amine in 250 ml of acetonitrile was added 2.073 g (15 mmol) of potassium carbonate. The reaction mixture was stirred for 3 days. After evaporation of the solvent the residue was treated with water, filtrated and dried to yield 4.0 g of 4-(2-chloropyridin-4-yl)-N-(6-methoxypyridin-3-yl)-1,3,5-triazin-2-amine (yield=84%) [M+1]=316.

### Step 2: Preparation of (4-{4-[(6-methoxypyridin-3-yl)amino]-1,3,5-triazin-2-yl}pyridin-2-yl)(piperidin-1-yl)-methanone (compound C-2)

300 mg of 4-(2-chloropyridin-4-yl)-N-(6-methoxypyridin-3-yl)-1,3,5-triazin-2-amine (0.95 mmol), 243 mg of piperidine (2.86 mmol), 252 mg of molybdenum hexacarbonyl (0.95 mmol), 0.427 ml of 1,8-Diazabicyclo(5.4.0)undec-7-ene (2.86 mmol) and 110 mg (0.095 mmol) of Tetrakis(triphenylphosphine)palladium(0) were diluted in 8 ml of N,N-dimethylformamide. The reaction mixture was stirred at 80° C. for 5 hours. After cooling, 10 ml of a saturated ammonium chloride solution was added and the mixture was extracted with 5 ml of dichloromethane. After evaporation of the solvent the crude product was chromatographed on silica (dichloromethane/ethanol) to yield 99 mg of (4-{4-[(6-methoxypyridin-3-yl)amino]-1,3,5-triazin-2-yl}pyridin-2-yl)(piperidin-1-yl)-methanone (yield=26%) [M+1]=393.

### Preparation of N-methoxy-N-methyl-4-[4-(3-thienylamino)-1,3,5-triazin-2-yl]pyridine-2-carboxamide (compound C-30) according to process P9

### Step 1: Preparation of methyl 4-[4-(3-thienylamino)-1,3,5-triazin-2-yl]pyridine-2-carboxylate (compound C-24) according to process P12

To a solution of 5 g (20 mmol) of methyl 4-(4-chloro-1,3,5-triazin-2-yl)pyridine-2-carboxylate (prepared as described in WO 2007/003525) and of 7.55 g (40 mmol) thiophen-3-ylamine oxalic acid salt in 150 ml of acetonitrile was added 5.5 g (40 mmol) of potassium carbonate. The reaction mixture was stirred for 7 hours at room temperature. The resulting suspension was filtered, the precipitate was washed with water followed by acetonitrile and finally diisopropylether to yield 3.7 g of methyl 4-[4-(3-thienylamino)-1,3,5-triazin-2-yl]pyridine-2-carboxylate (yield=58%) [M+1]=314.

### Step 2: Preparation of 4-[4-(3-thienylamino)-1,3,5-triazin-2-yl]pyridine-2-carboxylic acid (compound C-28)

2 g (6.38 mmol) of (methyl 4-[4-(3-thienylamino)-1,3,5-triazin-2-yl]pyridine-2-carboxylate

(obtained from step 1) was suspended in 20 ml tetrahydrofuran followed by the addition of 19 ml 1M lithium hydroxide. The mixture was stirred at room temperature for 30 minutes and then the tetrahydrofuran was evaporated. The resulting aqueous solution was kept overnight whereas a precipitate was formed which was removed by filtration. The filtrate was acidified to pH 2 with 2 N HCl. The precipitate formed was filtrated and dried to yield 1.9 g of 4-[4-(3-thienylamino)-1,3,5-triazin-2-yl]pyridine-2-carboxylic acid (yield=97%) [M+1]=300.

### Step 3: Preparation of N-methoxy-N-methyl-4-[4-(3-thienylamino)-1,3,5-triazin-2-yl]pyridine-2-carboxamide (compound C-30)

To a solution of 200 mg (0.67 mmol) of 4-[4-(3-thienylamino)-1,3,5-triazin-2-yl]pyridine-2-carboxylic acid (obtained from step 2) dissolved in 7 ml of dimethylformamide were added 203 mg (2 mmol) triethylamine, 130 mg (1.34 mmol) N,O-dimethylhydroxylamine hydrochloride and 305 mg (0.8 mmol) 0-(7-Azabenzotriazol-1-yl)-N,N,N′,N′-tetra-methyluronium hexafluorophosphate. The mixture was stirred at room temperature for 22 hours. After addition of water the mixture was extracted with dichloromethane and the organic layer was washed successively with 1 N HCl, saturated sodium bicarbonate and brine. After drying the solvent was to evaporated to yield 195 mg of N-methoxy-N-methyl-4-[4-(3-thienylamino)-1,3,5-triazin-2-yl]pyridine-2-carboxamide (yield=81%) [M+1]=343.

### BIOLOGICAL EXAMPLES

### Example A In Vivo Test on Peronospora parasitica (Crucifer Downy Mildew)

The active ingredients tested are prepared by homogenization in a mixture of acetone/Tween/DMSO, then diluted with water to obtain the desired active material concentration.

Cabbage plants (Eminence variety) in starter cups, sown on a 50/50 peat soil-pozzolana substrate and grown at 18-20° C., are treated at the cotyledon stage by spraying with the aqueous suspension described above.

Plants, used as controls, are treated with an aqueous solution not containing the active material. After 24 hours, the plants are contaminated by spraying them with an aqueous suspension of Peronospora parasitica spores (50 000 spores per ml). The spores are collected from infected plant.

The contaminated cabbage plants are incubated for 5 days at 20° C., under a humid atmosphere. Grading is carried out 5 days after the contamination, in comparison with the control plants. Under these conditions, good (at least 70%) or total protection is observed at a dose of 500 ppm with the following compounds: A5, All, B2, B6, B20, B27, B64, B76, B111, B116, B119, B123, B127, B129, B130, B132, B133, B134, B135, B139, B150, B151, B152, B156, B157, B158, B162, B163, B164, B165, B169, B170, B172, B173, B179, B180, B181, B186, B222, B236, B238, B239, B244, B254, B256, B258, B260, B268, B316, C2, C16.

### Example B

### In Vivo Test on Botrvtis cinerea (Grey Mould)

The active ingredients tested are prepared by homogenization in a mixture of acetone/Tween/DMSO, then diluted with water to obtain the desired active material Gherkin plants (Vert petit de Paris variety), sown on a 50/50 peat soil-pozzolana substrate in starter cups and grown at 18-20° C., are treated at the cotyledon Z11 stage by spraying with the active ingredient prepared as described above.

Plants, used as controls, are treated with an aqueous solution not containing the active material. After 24 hours, the plants are contaminated by depositing drops of an aqueous suspension of Botrytis cinerea spores (150,000 spores per ml) on upper surface of the leaves. The spores are collected from a 15-day-old culture and are suspended in a nutrient solution composed of:
20 g/L of gelatine;
50 g/L of D-fructose;
2 g/L of NH₄NO₃;
1 g/L of KH₂PO₄.

The contaminated cucumber plants are settled for 5/7 days in a climatic room at 15-11° C. (day/night) and at 80% relative humidity.

Grading is carried out 5/7 days after the contamination, in comparison with the control plants. Under these conditions, good (at least 70%) or total protection is observed at a dose of 500 ppm with the following compounds: A13, A15, A25, A26, A27, A29, A30, A31, A32, B1, B2, B6, B7, B20, B64, B115, B123, B124, B125, B127, B130, B131, B132, B133, B134, B135, B141, B143, B144, B146, B147, B148, B149, B150, B151, B152, B153, B155, B156, B157, B160, B161, B162, B163, B165, B169, B179, B186, B222, B236, B237, B238, B244, B254, B260, B268, B316, C5, C9, C11, C12, C16.

### Example C

### In Vivo Test on Alternaria brassicae (Leaf Spot of Crucifers)

The active ingredients tested are prepared by homogenization in a mixture of acetone/tween/DMSO, then diluted with water to obtain the desired active material.

Radish plants (Pernot variety), sown on a 50/50 peat soil-pozzolana substrate in starter cups and grown at 18-20° C., are treated at the cotyledon stage by spraying with the active ingredient prepared as described above.

Plants, used as controls, are treated with the mixture of acetone/tween/water not containing the active material.

After 24 hours, the plants are contaminated by spraying them with an aqueous suspension of Alternaria brassicae spores (40,000 spores per cm³). The spores are collected from a 12 to 13 days-old culture.

The contaminated radish plants are incubated for 6-7 days at about 18° C., under a humid atmosphere.

Grading is carried out 6 to 7 days after the contamination, in comparison with the control plants. Under these conditions, good protection (at least 70%) is observed at a dose of 500 ppm with the following compounds: B54, B55, B64, B91, B93, B94, B97, B115, B123, B124, B125, B127, B130, B131, B132, B133, B134, B135, B137, B138, B141, B142, B143, B144, B146, B148, B151, B152, B153, B156, B158, B159, B160, B161, B162, B165, B166, B167, B169, B171, B183, B184, B186.

### Example D

### In Vivo Test on Sphaerotheca fuliginea (Cucurbits Powdery Mildew)

The active ingredients tested are prepared by homogenization in a mixture of acetone/tween/DMSO, then diluted with water to obtain the desired active material.

Gherkin plants (Vert petit de Paris variety) in starter cups, sown on a 50/50 peat soil-pozzolana substrate and grown at 20° C./23° C., are treated at the cotyledon Z10 stage by spraying with the aqueous suspension described above. Plants, used as controls, are treated with an aqueous solution not containing the active material.

After 24 hours, the plants are contaminated by spraying them with an aqueous suspension of Sphaerotheca fuliginea spores (100 000 spores per ml). The spores are collected from a contaminated plants. The contaminated gherkin plants are incubated at about 20° C./25° C. and at 60/70% relative humidity.

Grading (% of efficacy) is carried out 12 days after the contamination, in comparison with the control plants.

Under these conditions, good (at least 70%) or total protection is observed at a dose of 500 ppm with the following compounds: A5, A18, A24, A26, A27, A29, A30, A32, B2, B6, B7, B20, B123, B127, B222, B225, B236, B239, B244, B254, B256, B258, B260, B268, C5, C10.

### Example E

### In Vivo Test on Pyrenophora teres (Barley Net Blotch)

The active ingredients tested are prepared by homogenization in a mixture of acetone/Tween/DMSO, then diluted with water to obtain the desired active material concentration.

Barley plants (Express variety), sown on a 50/50 peat soil-pozzolana substrate in starter cups and grown at 12° C., are treated at the 1-leaf stage (10 cm tall) by spraying with the active ingredient prepared as described above.

Plants, used as controls, are treated with an aqueous solution not containing the active material. After 24 hours, the plants are contaminated by spraying them with an aqueous suspension of Pyrenophora teres spores (12,000 spores per ml). The spores are collected from a 12-day-old culture. The contaminated barley plants are incubated for 24 hours at about 20° C. and at 100% relative humidity, and then for 12 days at 80% relative humidity.

Grading is carried out 12 days after the contamination, in comparison with the control plants.

Under these conditions, good (at least 70%) is observed at a dose of 500 ppm with the following compounds: A2, All, A17, A29, A30, A32, B1, B2, B6, B7, B20, B34, B36, B37, B38, B40, B41, B43, B45, B74, B75, B76, B77, B81, B82, B85, B105, B110, B111, B113, B115, B116, B123, B124, B125, B126, B127, B128, B129, B130, B131, B132, B133, B134, B135, B137, B139, B140, B141, B142, B143, B144, B145, B146, B147, B148, B149, B150, B151, B152, B153, B155, B156, B157, B158, B159, B160, B161, B162, B163, B164, B165, B166, B167, B169, B170, B171, B172, B173, B179, B180, B181, B183, B184, B186, B192, B207, B222, B237, B238, B239, B244, B252, B254, B256, B258, B260, B268, B316, C2, C4, C5, C7, C8, C9, C10, C11, C12, C16.

### Example F

### In Vivo Test on Puccinia recondita (Brown Rust)

The active ingredients tested are prepared by homogenization in a mixture of acetone/tween/DMSO, then diluted with water to obtain the desired active material. Wheat plants (Scipion variety) sown on 50/50 peat soil-pozzolana substrate in starter cups and grown at 12° C., are treated at the 1-leaf stage (10 cm tall) by spraying with the aqueous suspension described above.

Plants, used as controls, are treated with an aqueous solution not containing the active material.

After 24 hours, the plants are contaminated by spraying the leaves with an aqueous suspension of Puccinia recondita spores (100,000 spores per ml). The spores are collected from a 10-day-old contaminated wheat and are suspended in water containing 2.5 ml/l of tween 80 10%. The contaminated wheat plants are incubated for 24 hours at 20° C. and at 100% relative humidity, and then for 10 days at 20° C. and at 70% relative humidity.

Grading is carried out 10 days after the contamination, in comparison with the control plants. Under these conditions, good (at least 70%) or total protection is observed at a dose of 500 ppm with the following compounds: A5, A7, A17, A18, A25, A32, B1, B2, B6, B7, B111, B127, B129, B130, B132, B133, B146, B150, B151, B152, B157, B158, B159, B160, B161, B162, B163, B164, B165, B169, B170, B172, B186, B236, B237, B256, B316, C4, C5, C8, C9, C10, C11, C12, C16.

### Example G

### In Vivo Test on Mycosphaerella graminicola (Wheat Leaf Spot)

The active ingredients tested are prepared by homogenization in a mixture of acetone/tween/DMSO, then diluted with water to obtain the desired active material concentration.

Wheat plants (Scipion variety), sown on a 50/50 peat soil-pozzolana substrate in starter cups and grown at 12° C., are treated at the 1-leaf stage (10 cm tall) by spraying with the aqueous suspension described above. Plants, used as controls, are treated with an aqueous solution not containing the active material.

After 24 hours, the plants are contaminated by spraying them with an aqueous suspension of Mycosphaerella graminicola spores (500 000 spores per ml). The spores are collected from a 7-day-old culture. The contaminated wheat plants are incubated for 72 hours at 18° C. and at 100% relative humidity, and then for 21 to 28 days at 90% relative humidity.

Grading (% of efficacy) is carried out 21 to 28 days after the contamination, in comparison with the control plants.

Under these conditions, good (at least 70%) or total protection is observed at a dose of 500 ppm with the following compounds: B1, B2, B33, B45, B46, B48, B64, B75, B76, B81, B85, B95, B113, B114, B116, B118, B123, B124, B125, B126, B127, B129, B132, B133, B134, B135, B137, B146, B151, B157, B166, B186, B192, B194, B195, B197, B226, B227, C12.

### Example H

### Leptosphaeria Test (Wheat)/preventive

Solvent: 49 parts by weight of N,N-Dimethylformamide
Emulsifier: 1 part by weight of Alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with a preparation of active compound at the stated rate of application. One day after this treatment, the plants are inoculated with an aqueous spore suspension of Leptosphaeria nodorum. The plants remain for 48 hours in an incubation cabinet at 22° C. and a relative atmospheric humidity of 100%. Then the plants are placed in a greenhouse at a temperature of approximately 22° C. and a relative atmospheric humidity of approximately 90%.

The test is evaluated 7-9 days after the inoculation. 0% means an efficacy which corresponds to that of the control, while an efficacy of 100% means that no disease is observed.

In this test the following compounds according to the invention showed efficacy of 70% or even higher at a concentration of 500 ppm of active ingredient:
A38, B3, B4, B5, B8, B9, B10, B11, B12, B13, B14, B15, B16, B17, B18, B19, B21, B22, B23, B24, B25, B26, B27, B28, B29, B57, B64, B81, B115, B116, B123, B129, B132, B133, B136, B146, B151, B172, B187, B188, B189, B201, B202, B203, B204, B205, B206, B209, B223, B240, B241, B242, B243, B245, B246, B247, B248, B249, B250, B253, B255, B257, B259, B261, B262, B263, B264, B264, B265, B266, B267, B270, B271, B272, B273, B274, B275, B276, B277, B278, B279, B280, B281, B282, B283, B284, B285, B286, B287, B288, B289, B290, B291, B292, B293, B294, B295, B296, B297, B298, B299, B300, B301, B302, B303, B305, B307, B308, B310, B311, B312, B313, B314, B315, B317, B318, B326, B327, B328, B329, B330, B331, B332, B333, B334, B335, B336, B337, B338, B339, B340, B341, B342, B343, B344, B346, B347, B348, B349, B350, B352, B353, B354, B355, B356, B357, B358, B359, B361, B362, B363, B364, B365, B366, B367, B368, B369, B370, B371, B372, B373, B374, B375, B376, B377, B378, B379, B380, B381, B382, B383, B384, B385, B386, B387, B388, B389, B390, B391, B392, B393, B394, B395, B396, B397, B398, B399, B400, B401, C13, C14, C15, C17, C19, C20, C21, C22, C23.

### Example I

### Venturia Test (Apples)/protective

Solvent: 24.5 parts by weight of acetone
   24.5 parts by weight of dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. After the spray coating has dried on, the plants are inoculated with an aqueous conidia suspension of the causal agent of apple scab (Venturia inaequalis) and then remain for 1 day in an incubation cabinet at approximately 20° C. and a relative atmospheric humidity of 100%.

The plants are then placed in a greenhouse at approximately 21° C. and a relative atmospheric humidity of approximately 90%.

The test is evaluated 10 days after the inoculation. 0% means an efficacy which corresponds to that of the control, while an efficacy of 100% means that no disease is observed.

In this test the following compounds according to the invention showed efficacy of 70% or even higher at a concentration of 100 ppm of active ingredient:
A29, A30, A32, B2, B3, B4, B5, B5, B6, B7, B8, B9, B10, B11, B12, B13, B15, B16, B17, B19, B20, B23, B123, B129, B132, B133, B146, B151, B172, B186, B188, B203, B204, B205, B206, B209, B222, B223, B236, B238, B239, B248, B254, B255, B257, B261, B266, B268, B271, B272, B273, B274, B275, B276, B277, B278, B280, B281, B282, B285, B286, B287, B288, B289, B293, B297, B300, B310, B311, B312, B313, B315, B316, B318, B330, B331, B332, B333, B336, B337, B338, B339, B347, B349, B350, B355, B357, B358, B359, B362, B363, B364, B365, B366, B367, B368, B369, B370, B371, B372, B373, B374, B375, B379, B380, B381, B384, B386, B387, B389, B393, B393, B394, B395, B398, B399, B400, B401, C13, C14, C15, C16, C17, C19, C20, C21.

### Example J

### Uromyces Test (Beans)/protective

Solvent: 24.5 parts by weight of acetone
   24.5 parts by weight of dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. After the spray coating has dried on, the plants are inoculated with an aqueous spore suspension of the causal agent of bean rust (Uromyces appendiculatus) and then remain for 1 day in an incubation cabinet at approximately 20° C. and a relative atmospheric humidity of 100%.

The plants are then placed in a greenhouse at approximately 21° C. and a relative atmospheric humidity of approximately 90%.

The test is evaluated 10 days after the inoculation. 0% means an efficacy which corresponds to that of the control, while an efficacy of 100% means that no disease is observed.

In this test the following compounds according to the invention showed efficacy of 70% or even higher at a concentration of 100 ppm of active ingredient:
A29, A32, B2, B3, B4, B5, B6, B7, B8, B10, B11, B12, B13, B15, B16, B17, B19, B20, B23, B116, B123, B132, B133, B146, B151, B186, B209, B222, B223, B236, B238, B239, B254, B260, B261, B268, B271, B273, B274, B278, B281, B285, B286, B287, B289, B293, B295, B298, B301, B310, B311, B312, B313, B315, B316, B318, B330, B331, B332, B333, B336, B337, B338, B339, B347, B349, B350, B352, B364, B366, B367, B370, B371, B373, B374, B376, B380, B386, B390, B392, B393, B394, B395, B397, B398, B399, B400, C15, C16, C19, C21.

### Example K

### Pyricularia Test (Rice)/protective

Solvent: 28.5 parts by weight of acetone
Emulsifier: 1.5 parts by weight of polyoxyethylene alkyl phenyl ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. One day after spraying, the plants are inoculated with an aqueous spore suspension of the causal agent of rice blast (Pyricularia oryzae). The plants are then placed in an incubator at approximately 25° C. and a relative atmospheric humidity of approximately 100% for 1 day.

The test is evaluated 5 days after the inoculation. 0% means an efficacy which corresponds to that of the control, while an efficacy of 100% means that no disease is observed.

In this test the following compounds according to the invention showed efficacy of 80% or even higher at a concentration of 250 ppm of active ingredient:
B1, B2, B3, B4, B6, B7, B10, B81, B116, B123, B124, B125, B126, B127, B129, B132, B133, B134, B141, B151, B159, B160, B162, B169, B170, B172, B186, B187, B222, B240, B254, B256, B260, B266, B271, B278, B285, B286, B287, B288, B289, B313, B315, B318, B347, B349, B353, B359, B367, B373, B374, B375, B385, B389, B390, B394, B399, C9, C15, C16,

### Example L

### Rhizoctonia Test (Rice)/protective

Solvent: 28.5 parts by weight of acetone
Emulsifier: 1.5 parts by weight of polyoxyethylene alkyl phenyl ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. One day after spraying, the plants are inoculated with a hypha of the causal agent of rice sheath blight (Rhizoctonia solani). The plants are then placed in an incubator at approximately 25° C. and a relative atmospheric humidity of approximately 100%.

The test is evaluated 4 days after the inoculation. 0% means an efficacy which corresponds to that of the control, while an efficacy of 100% means that no disease is observed.

In this test the compounds according to the invention of the following structures showed efficacy of 80% or even higher at a concentration of 250 ppm of active ingredient:
B1, B2, B3, B4, B6, B7, B10, B20, B64, B74, B123, B124, B125, B126, B127, B129, B132, B133, B134, B135, B141, B142, B143, B144, B146, B147, B148, B149, B151, B156, B159, B160, B161, B162, B169, B170, B172, B186, B207, B238, B240, B260, B266, B271, B278, B285, B286, B287, B288, B289, B313, B315, B318, B347, B349, B353, B359, B367, B373, B374, B385, B389, B390, B392, B394, B395, B398, B399, C9, C15, C16.

### Example M

### Cochliobolus Test (Rice)/protective

Solvent: 28.5 parts by weight of acetone
Emulsifier: 1.5 parts by weight of polyoxyethylene alkyl phenyl ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. One day after spraying, the plants are inoculated with an aqueous spore suspension of the causal agent of rice brown spot (Cochliobolus miyabeanus). The plants are then placed in an incubator at approximately 25° C. and a relative atmospheric humidity of approximately 100% for 1 day.

The test is evaluated 4 days after the inoculation. 0% means an efficacy which corresponds to that of the control, while an efficacy of 100% means that no disease is observed.

In this test the following compounds according to the showed efficacy of 80% or even higher at a concentration of 250 ppm of active ingredient:
B1, B2, B3, B4, B6, B7, B10, B20, B64, B74, B116, B123, B124, B125, B126, B127, B129, B132, B133, B134, B135, B141, B142, B143, B144, B146, B147, B148, B151, B153, B156, B159, B160, B161, B162, B169, B170, B172, B186, B187, B238, B240, B254, B256, B260, B266, B268, B271, B278, B285, B286, B287, B288, B289, B313, B315, B318, B347, B349, B353, B359, B367, B373, B374, B375, B385, B389, B390, B392, B394, B395, B398, B399, C9, C15, C16.

### Example N

### Phakopsora Test (Soybeans)/protective

Solvent: 28.5 parts by weight of acetone
Emulsifier: 1.5 parts by weight of polyoxyethylene alkyl phenyl ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. One day after spraying, the plants are inoculated with an aqueous spore suspension of the causal agent of soybean rust (Phakopsora pachyrhizi). The plants are then placed in a greenhouse at approximately 20° C. and a relative atmospheric humidity of approximately 80%.

The test is evaluated 11 days after the inoculation. 0% means an efficacy which corresponds to that of the control, while an efficacy of 100% means that no disease is observed.

In this test the following compounds according to the showed efficacy of 80% or even higher at a concentration of 500 ppm of active ingredient:
A17, B3, B129, B132, B133, B186, B209, B287, B366, B371, C9.

### Example O

### Fusarium nivale (var. majus)-test (Wheat)/preventive

Solvent: 49 parts by weight of n,n-dimethylacetamid
Emulsifier: 1 part by weight of alkylaryl polyglycol ether

To produce a suitable preparation of active compound, 1 part by weight of active compound or active compound combination is mixed with the stated amounts of solvent and emulsifier, and the concentrate is diluted with water to the desired concentration.

To test for preventive activity, young plants are sprayed with the preparation of active compound or active compound combination at the stated rate of application. After the spray coating has dried on, the plants are sprayed with a spore suspension of Fusarium nivale (var. majus). The plants are placed in a greenhouse under translucent incubation cloches at a temperature of approximately 10° C. and a relative atmospheric humidity of approximately 100%.

The test is evaluated 5 days after the inoculation. 0% means an efficacy which corresponds to that of the control, while an efficacy of 100% means that no disease is observed.

In this test the following compounds according to the invention showed an efficacy of 70% or even higher at a concentration of 1000 ppm of active ingredient:
A29, A30, A38, B1, B2, B3, B4, B5, B6, B7, B8, B9, B10, B11, B12, B13, B14, B15, B16, B17, B18, B19, B21, B23, B81, B116, B123, B129, B132, B133, B146, B151, B172, B186, B201, B203, B204, B205, B206, B209, B223, B240, B241, B242, B245, B253, B255, B261, B266, B267, B271, B272, B273, B274, B277, B278, B284, B285, B286, B287, B288, B289, B290, B293, B295, B297, B299, B300, B314, B316, B317, B317, B318, B347, B349, B352, B353, B355, B359, B359, B373, B373, B374, B375, B376, B382, B385, B387, B388, B389, B390, B394, B397, B398, B399, C13, C13, C16, C17, C17, C19, C20, C21.

### Example P

### Puccinia triticina-test (Wheat)/preventive

Solvent: 49 parts by weight of n,n-dimethylacetamid
Emulsifier: 1 part by weight of alkylaryl polyglycol ether

To produce a suitable preparation of active compound, 1 part by weight of active compound or active compound combination is mixed with the stated amounts of solvent and emulsifier, and the concentrate is diluted with water to the desired concentration.

To test for preventive activity, young plants are sprayed with the preparation of active compound or active compound combination at the stated rate of application. After the spray coating has dried on, the plants are sprayed with a spore suspension of Puccinia triticina. The plants remain for 48 hours in an incubation cabinet at approximately 20° C. and a relative atmospheric humidity of approximately 100%. The plants are placed in a greenhouse at a temperature of approximately 20° C. and a relative atmospheric humidity of approximately 80%.

The test is evaluated 8 days after the inoculation. 0% means an efficacy which corresponds to that of the control, while an efficacy of 100% means that no disease is observed.

In this test the following compounds according to the invention showed an efficacy of 70% or even higher at a concentration of 1000 ppm of active ingredient:
A29, A32, A38, B1, B2, B3, B4, B5, B6, B7, B8, B9, B10, B11, B12, B13, B15, B16, B17, B18, B19, B123, B129, B132, B133, B146, B151, B172, B186, B201, B209, B247, B248, B261, B266, B272, B273, B274, B276, B278, B281, B284, B285, B286, B287, B288, B289, B290, B292, B293, B297, B299, B300, B314, B316, B317, B318, B347, B350, B352, B353, B355, B359, B364, B370, B371, B373, B374, B375, B376, B382, B385, B387, B389, B394, B397, B398, C13, C16, C17, C17, C19, C21.

1. A substituted-pyridyl-azinylamino derivative of formula (I) 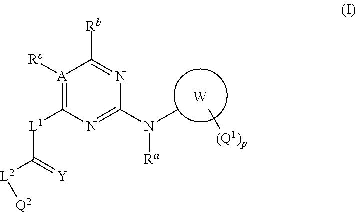
wherein
W represents phenyl or a saturated or unsaturated, aromatic or non-aromatic 4-, 5-, 6- or 7-membered heterocycle comprising up to four heteroatoms which may be the same or different
A represents a carbon atom or a nitrogen atom provided that if A represents a carbon atom then W represents a saturated or unsaturated, aromatic or non-aromatic 4-, 5-, 6- or 7-membered heterocycle comprising up to four heteroatoms which may be the same or different
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈alkenyloxy, a C₂-C₈alkynyloxy, a C₁-C₈alkylamino, a di-C₁-C₈alkylamino, a C₁-C₈-alkoxy, a C₁-C₈halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino; it being possible for each of these groups or substituents to be substituted when chemically possible;
p represents 0, 1, 2, 3, 4 or 5;
R^{a} represents a hydrogen atom, a cyano group, a formyl group, a formyloxy group, a C₁-Cₛ-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, or a C₁-C₈-halogenoalkoxyalkyl having 1 to 5 halogen atoms, a C₁-C₈-alkoxyalkylcarbonyl, a C₁-C₈-halogenoalkoxyalkycarbonyl having 1 to 5 halogen atoms, a C₁-C₈alkylthioalkylcarbonyl, a C₁-C₈-halogenoalkylthioalkylcarbonyl having 1 to 5 halogen atoms; it being possible for each of these groups or substituents to be substituted when chemically possible;
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, or a C₁-C₈-halogenocycloalkyl having 1 to 5 halogen atoms; it being possible for each of these groups or substituents to be substituted when chemically possible;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl-C₁-C₈ alkyl, C₁-C₈-cycloalkyl, tri(C₁-C₈alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈alkylaminosulfamoyl, di-C₁-C₈alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible;
Alternatively, L2 and Q2 can form together a substituted or non-substituted 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, tri(C₁-C₈-alkyl)silyl-C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈alkylcarbonyl, C₁-C₈-halogenoalkylcarbonyl having 1 to 5 halogen atoms, C₁-C₈alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈alkylcarbonyloxy, C₁-C₈-halogenoalkylcarbonyloxy having 1 to 5 halogen atoms, C₁-C₈alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, C₁-C₈-alkylsulphinyl, C₁-C₈-halogenoalkylsulphinyl having 1 to 5 halogen atoms, C₁-C₈-alkylsulphonyl, C₁-C₈-halogenoalkylsulphonyl having 1 to 5 halogen atoms, C₁-C₈-alkylaminosulfamoyl, di-C₁-C₈-alkylaminosulfamoyl, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible;
Unless indicated otherwise, a group or a substituent that is substituted is substituted by one or more of the following groups or atoms: a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈alkylaminosulfamoyl, a di-C₁-C₈alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino;
as well as salts, N-oxides, metallic complexes, metalloïdic complexes and optically active or geometric isomers thereof; provided that the following compounds are excluded:
(3-{[4-(2-ethenylpyridin-4-yl)-1,3,5-triazin-2-yl]amino}phenyl)methanol;
methyl 4-{4-[(3-{2-[(tert-butoxycarbonyl)amino]ethoxy}phenyl)amino]-1,3,5-triazin-2-yl}-pyridine-2-carboxylate;
N-{3-[(4-{2-[(1Z)-3-amino-2-chloroprop-1-en-1-yl]pyridin-4-yl}-1,3,5-triazin-2-yl)amino]-benzyl}-N-methylglycine.

2. A compound of formula (I) according to claim 1 wherein W represents a phenyl and A represents a nitrogen atom.

3. A compound of formula (I) according to claim 1 wherein W represents a a saturated or unsaturated, aromatic or non-aromatic 4-, 5-, 6- or 7-membered heterocycle comprising up to four heteroatoms which may be the same or different.

4. A compound of formula (I) according to claim 1 wherein A represents a nitrogen atome and W represents a a saturated or unsaturated, aromatic or non-aromatic 4-, 5-, 6- or 7-membered heterocycle comprising up to four heteroatoms which may be the same or different.

5. A compound of formula (I) according to claim 1 wherein A represents a carbon atom and W represents a saturated or unsaturated, aromatic or non-aromatic 4-, 5-, 6- or 7-membered heterocycle comprising up to four heteroatoms which may be the same or different.

6. A compound of formula (I) according to claim 1 wherein W represents phenyl or a saturated or unsaturated, aromatic or non-aromatic heterocycle selected in the list consisting of:

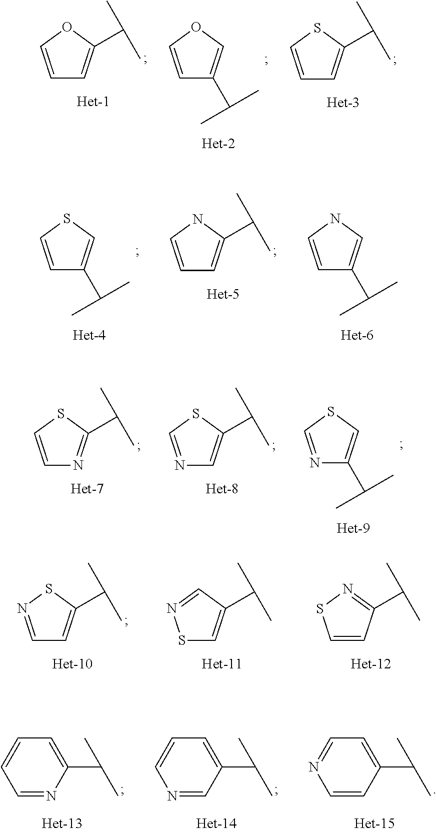

7. A compound of formula (I) according to claim 1 wherein Q¹ represents a halogen atom, a nitro group, a hydroxy group, a cyano group, an amino group, a sulfanyl group, a pentafluoro-λ⁶-sulfanyl group, a formyl group, a formyloxy group, a formylamino group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₁-C₈-alkylcarbonyl, a C₁-C₈-halogenoalkylcarbonyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a (C₁-C₆-alkoxyimino)-C₁-C₆-alkyl, C₁-C₈alkoxyalkyl, C₁-C₈-halogenoalkoxyalkyl having 1 to 5 halogen atoms; it being possible for each of these groups or substituents to be substituted when chemically possible.

8. A compound of formula (I) according to claim 1 wherein p represents 0, 1, 2, or 3.

9. A compound of formula (I) according to claim 1 wherein p represents 1.

10. A compound of formula (I) according to claim 1 wherein R^{a} represents a hydrogen atom or a substituted or non substituted C₁-C₈-cycloalkyl.

11. A compound of formula (I) according to claim 1 wherein R^{b} and R^{c} independently represent a hydrogen atom, a halogen atom, a cyano, a C₁-C₈-halogenoalkyl having 1 to 5 halogen atoms, a C₁-C₈-halogenocycloalkyl having 1 to 5 halogen atoms.

12. A compound of formula (I) according to claim 1 wherein R^{b} and R^{c} independently represent a hydrogen atom or a halogen atom.

13. A compound of formula (I) according to claim 1 wherein R^{d} to Rⁱ independently represent a hydrogen atom, a halogen atom, a nitro group, a cyano group, a hydroxy group, an amino group, a sulfanyl group, a formyl group, a formyloxy group, a formylamino group, (hydroxyimino)-C₁-C₆-alkyl group, C₁-C₈-alkyl, tri(C₁-C₈-alkyl)silyl, tri(C₁-C₈-alkyl)silyl-C₁-C₈-alkyl, C₁-C₈-cycloalkyl, C₁-C₈-halogenoalkyl having 1 to 5 halogen atoms, C₁-C₈-halogenocycloalkyl having 1 to 5 halogen atoms a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₂-C₈-alkenyloxy, C₂-C₈-alkynyloxy, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-C₁-C₈-alkylaminocarbonyloxy, C₁-C₈-alkyloxycarbonyloxy, C₁-C₈-alkylsulphenyl, C₁-C₈-halogenoalkylsulphenyl having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₆-alkyl, phenylamino, phenyl hetarylamino, or a 4-, 5-, 6- or 7-membered heterocycle comprising up to 4 heteroatoms selected in the list consisting of N, O, S; it being possible for each of these groups or substituents to be substituted when chemically possible.

14. A compound of formula (I) according to claim 1 wherein R^{d} represents H, (methoxycarbonyl)amino, (4-chlorophenyl)amino, [3-chloro-5-(trifluoromethyl)pyridin-2-yl]amino, (2-ethoxy-2-oxoethyl)amino, (2,2,2-trifluoroethyl)amino, (2-cyanoethyl)amino, methylamino, (2-methylpropanoyl)oxy, (3-methylbut-2-enoyl)oxy, (3-methylbutanoyl)oxy, butanoyloxy, propanoyloxy, (methoxyacetyl)oxy, acetyloxy, cyclopentyloxy, dicyclopropylmethoxy, 1-cyclopropylethoxy, but-3-yn-2-yloxy, hex-2-yn-1-yloxy, but-2-yn-1-yloxy, prop-2-yn-1-yloxy, 2,2,2-trifluoroethoxy, (2,6-dichlorobenzyl)oxy, (4-chlorobenzyl)oxy, (4-methoxybenzyl)oxy, benzyloxy, cyclopropylmethoxy, 2-methylpropoxy, prop-2-en-1-yloxy, propoxy, 2-(dimethylamino)ethoxy, ethoxy, methoxy, hydroxyl, phenylamino, or phenyl hetarylamino.

15. A compound of formula (I) according to claim 1 wherein R^{g} represents Hydrogen, prop-2-en-1-yl, hexyl, butyl, propyl, 2-hydroxyethyl, ethyl, methyl.

16. A compound of formula (I) according to claim 1 wherein L¹ is selected in the list consisting of: 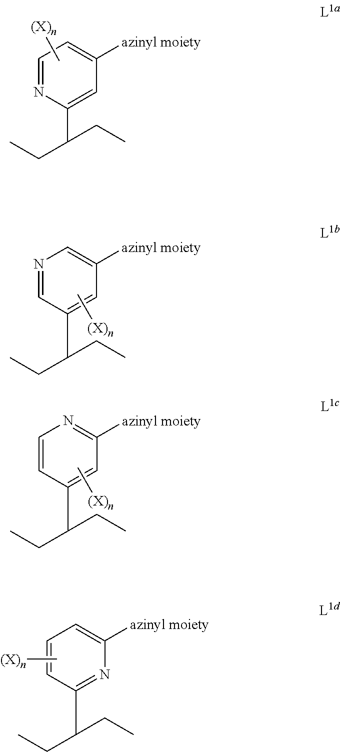
wherein
n represents 0, 1, 2 or 3;
X independently represents a C₁-C₁₀-alkyl, a C₁-C₁₀-halogenoalkyl, a halogen atom or a cyano.

17. A compound of formula (I) according to claim 1 wherein L¹ is selected in the list consisting of: 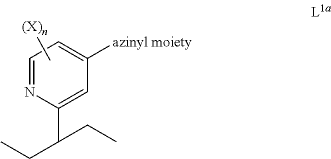
wherein
n represents 0, 1, 2 or 3;
X independently represents a C₁-C₁₀-alkyl, a C₁-C₁₀-halogenoalkyl, a halogen atom or a cyano.

18. A compound of formula (I) according to claim 1 wherein Q² represents a hydrogen atom, a halogen atom, a hydroxy group, a cyano group, an amino group, a sulfanyl group, a formyl group, a formyloxy group, a formylamino group, a carbamoyl group, a N-hydroxycarbamoyl group, a carbamate group, (hydroxyimino)-C₁-C₆-alkyl group, C₁-C₈-alkyl, C₁-C₈-cycloalkyl, C₁-C₈-halogenoalkyl having 1 to 5 halogen atoms, a C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-alkylcarbonyl, C₁-C₈-halogenoalkylcarbonyl having 1 to 5 halogen atoms, C₁-C₈-halogenoalkoxycarbonyl having 1 to 5 halogen atoms, C₁-C₈-alkylcarbonylamino, C₁-C₈-halogenoalkylcarbonylamino having 1 to 5 halogen atoms, (C₁-C₆-alkoxyimino)-C₁-C₆-alkyl, (C₁-C₆-alkenyloxyimino)-C₁-C₆-alkyl, (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, (benzyloxyimino)-C₁-C₈-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, phenylamino, a or a 4-, 5-, 6- or 7-membered heterocycle comprising up to 4 heteroatoms selected in the list consisting of N, O, S, or a (4-, 5-, 6- or 7-membered heterocyclyl) C₁-C₆-alkyl comprising up to 4 heteroatoms selected in the list of consisting of N, O, S; it being possible for each of these groups or substituents to be substituted when chemically possible; or L² and Q² form together a, 4-, 5-, 6- or 7-membered non-aromatic heterocycle comprising up to 4 heteroatoms selected in the list consisting of N, O, S.

19. A compound of formula (I) according to claim 1 wherein Q² represents (2R)-2-(methoxymethyl)pyrrolidin-1-yl, (2S)-1-methoxypropan-2-yl, 1-(diethylamino)propan-2-yl, 1-(dimethylamino)propan-2-yl, 1,1-dioxidotetrahydrothiophen-3-yl, 1,3-dimethoxypropan-2-yl, 1-cyanobutan-2-yl, 1-cyclopropyl-2-methoxyethyl, 1-ethylpiperidin-3-yl, 1-methoxybutan-2-yl, 1-methoxypropan-2-yl, 2-(hydroxymethyl)piperidin-1-yl, 2-(morpholin-4-yl)ethyl, 2,2,2-trifluoroethyl, 2,3-dimethylpiperidin-1-yl, 2,5-dimethylpyrrolidin-1-yl, 2,6-dimethylmorpholin-4-yl, 2-cyanoethyl, 2-ethylpiperidin-1-yl, 2-hydroxy-2-methylpropyl, 2-hydroxyethyl, 2-methoxyethyl, 2-methylpiperidin-1-yl, 2-methylprop-2-en-1-yl, 2-methylpropyl, 2-methylpyrrolidin-1-yl, 3-(2-oxoazepan-1-yl)propyl, 3-(2-oxopyrrolidin-1-yl)propyl, 3-(formylamino)propyl, 3-(hydroxymethyl)piperidin-1-yl, 3,3,3-trifluoropropyl, 3,3-dimethylpiperidin-1-yl, 3,5-dimethylpiperidin-1-yl, 3,6-dihydropyridin-1(2H)-yl, 3-hydroxypiperidin-1-yl, 3-hydroxypropyl, 3-hydroxypyrrolidin-1-yl, 3-methoxybutan-2-yl, 3-methoxypiperidin-1-yl, 3-methoxypropyl, 3-methylbut-2-en-1-yl, 3-methylbutan-2-yl, 3-methylbutyl, 3-methylpiperidin-1-yl, 4-(2-oxopyrrolidin-1-yl)butyl, 4-(trifluoromethyl)piperidin-1-yl, 4-cyanopiperidin-1-yl, 4-ethoxycyclohexyl, 4-formylpiperazin-1-yl, 4-hydroxypiperidin-1-yl, 4-methoxypiperidin-1-yl, 4-methylpiperazin-1-yl, 4-methylpiperidin-1-yl, 4-oxoimidazolidin-1-yl, azepan-1-yl, butan-2-yl, butyl, cyclobutyl, cyclohexyl, cyclopentyl, cyclopropyl, cyclopropylmethyl, ethyl, hydrogen, hexyl, hydroxy, methoxy, methyl, morpholin-4-yl, oxetan-3-yl, pentan-2-yl, pentan-3-yl, pentyl, piperidin-1-yl, prop-2-en-1-yl, propan-2-yl, propyl, pyrrolidin-1-yl, tert-butyl, tetrahydrofuran-2-ylmethyl, thiomorpholin-4-yl.

20. A compound of formula (V) useful as intermediate compound or material for the preparation of a compound of formula (I) according to claim 1.

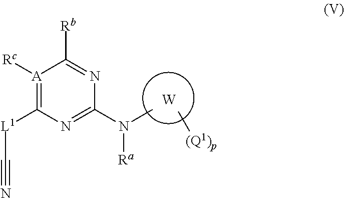

21. A compound of formula (VI) useful as intermediate compound or material for the preparation of a compound of formula (I) according to claim 1 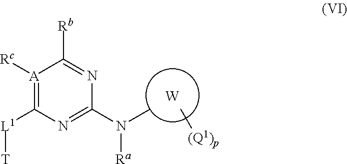
wherein T represents a chlorine atom, provided that the following compounds are excluded:
N-(3-chlorophenyl)-4-(2-chloropyridin-4-yl)-1,3,5-triazin-2-amine
4-(2-chloropyridin-4-yl)-N-[3-(1,1,2,2-tetrafluoroethoxy)phenyl]-1,3,5-triazin-2-amine
4-(2-chloropyridin-4-yl)-N-(3,4,5-trimethoxyphenyl)-1,3,5-triazin-2-amine.

22. A compound of formula (VI) according to claim 21 selected from the group constituted of 4-(2-chloropyridin-4-yl)-N-(pyridin-3-yl)pyrimidin-2-amine, 4-(2-chloropyridin-4-yl)-N-(6-methoxypyridin-3-yl)-1,3,5-triazin-2-amine, 4-(2-chloropyridin-4-yl)-N-(3,4,5-trimethoxyphenyl)-1,3,5-triazin-2-amine, 3-{[4-(2-chloropyridin-4-yl)-1,3,5-triazin-2-yl]amino}benzoic acid, 4-(2-chloropyridin-4-yl)-N-(6-chloropyridin-2-yl)pyrimidin-2-amine, N,4-bis(2-chloropyridin-4-yl)pyrimidin-2-amine, N⁴-[4-(2-chloropyridin-4-yl)pyrimidin-2-yl]-N²-(1-methoxybutan-2-yl)pyridine-2,4-diamine, N-(3-chloro-4-fluorophenyl)-4-(2-chloropyridin-4-yl)-1,3,5-triazin-2-amine, 4-(2-chloropyridin-4-yl)-N-[3-(methylsulfanyl)phenyl]-1,3,5-triazin-2-amine, N-(3-chloro-4-methylphenyl)-4-(2-chloropyridin-4-yl)-1,3,5-triazin-2-amine, N⁴-[4-(2-chloropyridin-4-yl)pyrimidin-2-yl]-N²-cyclobutylpyridine-2,4-diamine, N⁴-[4-(2-chloropyridin-4-yl)pyrimidin-2-yl]-N²-(3-methylbutan-2-yl)pyridine-2,4-diamine, N⁴-[4-(2-chloropyridin-4-yl)pyrimidin-2-yl]-N²-(pentan-3-yl)pyridine-2,4-diamine, 4-(2-chloropyridin-4-yl)-N-(pyridin-4-yl)-1,3,5-triazin-2-amine, methyl 3-{[4-(2-chloropyridin-4-yl)-1,3,5-triazin-2-yl]amino}thiophene-2-carboxylate, ethyl 2-{[4-(2-chloropyridin-4-yl)-1,3,5-triazin-2-yl]amino}-4-methyl-1,3-thiazole-5-carboxylate, 4-(2-chloropyridin-4-yl)-N-(4-methyl-1,3-thiazol-2-yl)-1,3,5-triazin-2-amine, 4-(2-chloropyridin-4-yl)-N-(5-methyl-1,3-thiazol-2-yl)-1,3,5-triazin-2-amine, 4-(2-chloropyridin-4-yl)-N-(2-methylpyridin-4-yl)pyrimidin-2-amine, N-(2-bromopyridin-4-yl)-4-(2-chloropyridin-4-yl)pyrimidin-2-amine, N-(5-bromopyridin-3-yl)-4-(2-chloropyridin-4-yl)pyrimidin-2-amine, 4-(2-chloropyridin-4-yl)-N-[2-(trifluoromethyl)pyridin-4-yl]pyrimidin-2-amine, 2-{[4-(2-chloropyridin-4-yl)-1,3,5-triazin-2-yl]amino}thiophene-3-carbonitrile, N-(5-chloro-3-methylpyridin-2-yl)-4-(2-chloropyridin-4-yl)-1,3,5-triazin-2-amine, 4-(2-chloropyridin-4-yl)-N-(4-chloropyridin-3-yl)-1,3,5-triazin-2-amine, 4-(2-chloropyridin-4-yl)-N-(2-methylpyridin-4-yl)-1,3,5-triazin-2-amine, N,4-bis(2-chloropyridin-4-yl)-N-(methoxymethyl)pyrimidin-2-amine, 4-(2-chloropyridin-4-yl)-N-(2,5-difluorophenyl)-1,3,5-triazin-2-amine, 4-(2-chloropyridin-4-yl)-N-(3-fluorophenyl)-1,3,5-triazin-2-amine, 4-(2-chloropyridin-4-yl)-N-[3-(methoxymethyl)phenyl]-1,3,5-triazin-2-amine, 4-(2-chloropyridin-4-yl)-N-(thiophen-3-yl)-1,3,5-triazin-2-amine, 4-(2-chloropyridin-4-yl)-N-[3-(trifluoromethyl)phenyl]-1,3,5-triazin-2-amine, 4-(2-chloropyridin-4-yl)-N-[3-(propan-2-yl)phenyl]-1,3,5-triazin-2-amine, 4-(2-chloropyridin-4-yl)-N-[3-(1,1,2,2-tetrafluoroethoxy)phenyl]-1,3,5-triazin-2-amine, 4-(2-chloropyridin-4-yl)-N-[3-(trifluoromethoxy)phenyl]-1,3,5-triazin-2-amine, 4-(2-chloropyridin-4-yl)-N-[3-(pentafluoro-lambda⁶-sulfanyl)phenyl]-1,3,5-triazin-2-amine, 4-(2-chloropyridin-4-yl)-N-(3-ethoxyphenyl)-1,3,5-triazin-2-amine, 4-(2-chloropyridin-4-yl)-N-(3-methoxyphenyl)-1,3,5-triazin-2-amine, 4-(2-chloropyridin-4-yl)-N-phenyl-1,3,5-triazin-2-amine, 4-(2-chloropyridin-4-yl)-N-(4-fluorophenyl)-1,3,5-triazin-2-amine, 4-(2-chloropyridin-4-yl)-N-[3-(difluoromethoxy)phenyl]-1,3,5-triazin-2-amine, N-(3-{[4-(2-chloropyridin-4-yl)-1,3,5-triazin-2-yl]amino}phenyl)acetamide, and 4-(2-chloropyridin-4-yl)-N-[3-(difluoromethyl)phenyl]-1,3,5-triazin-2-amine.

23. The use of the compound of claim 20 as an intermediate for the preparation of a compound of formula (I):

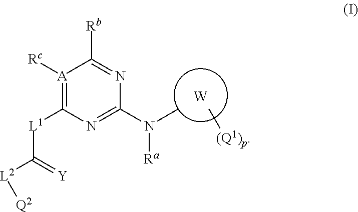

24. A fungicide composition comprising, as an active ingredient, an effective amount of a compound according to claim 1 and an agriculturally acceptable support, carrier or filler.

25. A method for controlling phytopathogenic fungi of crops, characterized in that an agronomically effective and substantially non-phytotoxic quantity of a compound according to claim 1 is applied to the soil where plants grow or are capable of growing, to the leaves and/or the fruit of plants or to the seeds of plants.

26. A method for combating phytopathogenic and mycotoxin producing fungi characterized in that a compound according to claim 1 is applied to these fungi and/or their habitat.

27. A method for controlling phytopathogenic fungi of crops, characterized in that an agronomically effective and substantially non-phytotoxic quantity of a composition according to claim 24 is applied to the soil where plants grow or are capable of growing, to the leaves and/or the fruit of plants or to the seeds of plants.

28. A method for combating phytopathogenic and mycotoxin producing fungi characterized in that a composition according to claim 24 is applied to these fungi and/or their habitat.
